(19) United States
(12) Patent Application Publication
(10) Pub. No.: US 2014/0081012 A1
(43) Pub. Date: Mar. 20, 2014

(54) NANOPARTICLE, LIPOSOMES, POLYMERS, AGENTS AND PROTEINS MODIFIED WITH REVERSIBLE LINKERS

(71) Applicants: Joseph M. DeSimone, Chapel Hill, NC (US); Mary Napier, Chapel Hill, NC (US); Jin Wang, Houston, TX (US); Jing Xu, Chapel Hill, NC (US); Shaomin Tian, Chapel Hill, NC (US); Stuart Dunn, Carrboro, NC (US)

(72) Inventors: Joseph M. DeSimone, Chapel Hill, NC (US); Mary Napier, Chapel Hill, NC (US); Jin Wang, Houston, TX (US); Jing Xu, Chapel Hill, NC (US); Shaomin Tian, Chapel Hill, NC (US); Stuart Dunn, Carrboro, NC (US)

(73) Assignee: THE UNIVERSITY OF NORTH CAROLINA AT CHAPEL HILL, Chapel Hill, NC (US)

(21) Appl. No.: 13/985,491

(22) PCT Filed: Feb. 15, 2012
(86) PCT No.: PCT/US12/25260

§ 371 Date: Oct. 23, 2013

Related U.S. Application Data
(60) Provisional application No. 61/442,951, filed on Feb. 15, 2011.

(51) Int. Cl. C07D 207/46; C12N 15/113

(52) U.S. Cl. CPC C07D 207/46; C12N 15/113
USPC 536/24.5; 548/542

(57) ABSTRACT

Pharmaceutical, chemical and biological agents containing a reversible disulfide linker are described. These agents can also be covalently bound or contained in delivery vehicles for delivering the agents to desired targets or areas. Also described are delivery vehicles which contain an agent having a reversible disulfide linker and to vehicles that are covalently linked to the agent containing a reversible disulfide linker. The modifications described herein can modify properties of the agents and vehicles, thereby providing desired solubility, stability, hydrophobicity and targeting while the reversibility of the linker can leave the agent to which it is attached free from residual chemical groups after being reduced.

### GOVERNMENT SUPPORT

This invention was made with government support under Grants 1-R01-EB009565, 1-DP1-OD006432 and U54-CA119343 awarded by the National Institutes of Health. The government has certain rights in the invention.

### FIELD OF THE INVENTION

The subject matter herein is directed to polymers, proteins, nanoparticles and liposomes that contain reversible linker(s).

### BACKGROUND

Drug delivery technology has been exploited extensively for the purpose of delivering agents to desired targets for many years. Drug delivery technologies involve conjugation chemistries, emulsion particles, liposomes and nano or microparticles. Hydrophobic or hydrophilic compounds can be entrapped in the hydrophobic domain or encapsulated in the aqueous compartment, respectively. Liposomes can be constructed of natural constituents so that the liposome membrane is in principal identical to the lipid portion of natural cell membranes. It is considered that liposomes are quite compatible with the human body when used as drug delivery systems.

The cellular delivery of various therapeutic compounds, such as chemotherapeutic agents, is usually compromised by two limitations. First, the selectivity of a number of therapeutic agents is often low, resulting in high toxicity to normal tissues. Secondly, the trafficking of many compounds into living cells is highly restricted by the complex membrane systems of the cell. Specific transporters allow the selective entry of nutrients or regulatory molecules, while excluding most exogenous molecules such as nucleic acids and proteins.

The problems mentioned above are not adequately addressed by existing delivery vehicles or compositions. The presently disclosed subject matter addresses, in whole or in part, these and other needs in the art.

### SUMMARY OF THE INVENTION

In an embodiment, the present subject matter is directed to nanoparticles, polymers, proteins and liposomes comprising reversible linkers. In some embodiments the reversible linker is a disulfide linker and in further embodiments the reversible linker has a trityl moiety, an ester moiety, or a CDM (carboxylated dimethyl maleic acid) moieties.

In an embodiment, the present subject matter is directed to methods of modifying a nanoparticle, polymer or liposome by contacting the nanoparticle, polymer or liposome with a molecule comprising one or more reversible disulfide linkers.

In an embodiment, the present subject matter is directed to nanoparticles, polymers or liposomes comprising a therapeutic agent that is covalently linked to a reversible disulfide linker.

In an embodiment, the present subject matter is directed to methods of delivering an active agent comprising administering to a subject the nanoparticles or liposomes disclosed herein.

In an embodiment, a pharmaceutical, chemical or biological agent is covalently linked to a reversible disulfide linker.

In an embodiment, the present subject matter is directed to reversible disulfide linkers useful for modifying therapeutic agents, polymers, nanoparticles and liposomes.

### BRIEF DESCRIPTION OF THE FIGURE

FIG. 1 depicts the hydrolysis of reversible disulfide linkages under reducing conditions. Upon hydrolysis, the particle and/or the composition or component thereof has no remnant of the linker, i.e., the particle and/or component has the same structure as before conjugation with the linker.

FIG. 2 depicts several reversible disulfide linkers as disclosed herein.

FIGS. 3 and 4 depict several therapeutic agents comprising a reversible disulfide linker as described herein.

FIG. 5 depicts the dissolution profile of reversible disulfide linked particles in PBS buffer vs. PBS with 5 mM glutathione. The fluorescence (excitation 545 nm, emission 575 nm) was measured by a SpectraMax M5 plate reader (Molecular Devices). The fluorescence from PBS was used as background and the fluorescence from uncrosslinked particles (0.25 mg/mL in PBS) was used as 100% control. Crosslinked particles that were exposed to PBS only remained intact over the 48 hr time period, while the particles exposed to PBS with the reducing agent glutathione were fully degraded at 48 hours.

FIG. 6 shows an ESEM image of particles cross-linked in IPA for 24 hours. The particles that were crosslinked with reversible reversible disulfide crosslinker Dithio-bis(ethyl 1H-imidazole-1-carboxylate) (DIC) for 24 hours at 37° C. The image was taken after incubation with water. Particles remain intact following incubation in water.

FIG. 7 depicts cell uptake of reversible disulfide containing particles by confocal laser scanning microscopy image of particles on HeLa cells. A: Particles with no PEI; B: Particles with 2 wt % of PEI; C: Particles with 4 wt % of PEI.

FIGS. 8 A and B depict the knockdown of luciferase in HeLa cells was observed following dosing of amine terminated anti-luciferase siRNA or anti-luciferase siRNA pro-drug transfected using lipofectamine.

FIG. 9 depicts a graph showing knockdown of luciferase expression as a function of siRNA concentration. Knockdown of luciferase expression was observed for reversible disulfide pro-drug siRNA particles containing the anti-luciferase siRNA and 20 or 50% AEM. 30% knockdown of luciferase expression was observed with hydrogels containing 20 wt % AEM, and >90% knockdown was observed with 50 wt % AEM. No knockdown was observed for the particle containing anti-luciferase siRNA5 wt % AEM. No knockdown was observed for irrelevant control.

FIGS. 10 A, B, C and D depict luciferase in HeLa cells and cell viability. Orange bars (bars on the right) indicate the amount of luciferase expression observed when compared to the controls as a function of dosed particle concentration. Blue bars (bars on the left) indicate the cell viability as a function of particle concentration.

FIG. 11A depicts an exemplary reversible linker having a trityl moiety; FIG. 11B depicts an exemplary reversible linker having an ester moiety; and Figure C depicts an exemplary reversible linker having a CDM (carboxylated dimethyl maleic acid) moieties; DDV (drug delivery vehicle).

FIGS. 12A, B & C: (a) Reaction scheme for PEGylation of hydrogels with succinimidyl succinate monomethoxy PEG_{2K}, (b) time-dependent release of siRNA from particles incubated at 2 mg/mL and 37° C. in PBS 1.4 wt % loading, and (c) SEM of particles illustrating their 200×200 nm cylindrical dimensions (scale bar=2 μm).

FIGS. 13A & B: (a) Cellular uptake and (b) luciferase expression of HeLa/luc cells dosed with PEGylated hydrogels containing different siRNA cargos. Cells were dosed with particles for 4 h followed by removal of particles and 72 h incubation in media.

FIG. 14 depicts viability of HeLa cell dosed with PEGylated, siRNA-containing hydrogels. Cells were dosed with particles for 4 h and incubated for 72 h in media.

FIG. 15 depicts time-dependent release of siRNA from hydrogels after post-fabrication functionalization with targeting ligands when incubated at 2 mg/mL and 37° C. in PBS demonstrates loss of physically entrapped cargo (0.7 wt % encapsulated compared to 1.4 wt % originally encapsulated).

FIGS. 16A, B & C: (a) Structures of degradable and control siRNA macromers, (b) SEM micrograph of pro-siRNA, 200×200 nm cylindrical nanoparticles (scale bar=2 μm), and (c) Illustration of pro-siRNA hydrogel behavior under physiological and intracellular conditions.

FIGS. 17 A, B & C depict release profiles and stability of siRNA in 30% AEM-based hydrogels: (a) Time-dependent incubation of pro-siRNA hydrogels (1 mg/mL) in PBS and under reducing conditions (glutathione, 5 mM) at 37° C. (b) Reductively-triggered release of siRNA prodrug from hydrogels (different cargo abbreviations listed below). Hydrogels were incubated in 10×PBS with or without 5 mM glutathione for 4 h at 1 mg/mL and 37° C. (c) Retention of siRNA integrity when conjugated to hydrogels after exposure to 10% FBS over time. Naked siRNA PD macromer was incubated at 36 ug/mL in 10% FBS for given times, proceeded by storage of solution. pro-siRNA hydrogels were incubated at 1.2 mg/mL in 10% FBS at 37° C. for given times followed by incubation in 10×PBS (5 mM glutathione) for 4 h at 1.2 mg/mL and 37° C. to release siRNA. Differences in siRNA migration observed in gels among the standards and samples which were released from hydrogels incubated in PBS and 10× PBS may arise from the differences in salt concentrations of sample solutions. AA (Fire, A.; Xu, S.; Montgomery, M. K.; Kostas, S. A.; Driver, S. E.; Mello, C. C. Nature 1998, 391, 806-811): acrylamide non-degradable siRNA prodrug; NH₂ (Elbashir, S. M.; Lendeckel, W.; Tuschl, T. Genes Dev. 2001, 15, 188-200): native amine siRNA; PD (Leuschner, F. et al. Nat. Biotechnol. 2011, 1-9): degradable siRNA prodrug.

FIG. 18 depicts gel electrophoresis of siRNA released from hydrogels prepared with different amine monomer contents. Hydrogels were incubated in 10×PBS containing 5 mM glutathione at 1.7 mg/mL for 4 h at 37° C.

FIGS. 19A & B: (a) Luciferase expression and (b) viability of HeLa/luc cells dosed with cationic pro-siRNA hydrogels fabricated with different amine (AEM) contents. Cells were dosed with particles for 5 h followed by removal of particles and 48 h incubation in media. Half maximal effective concentrations (EC₅₀5) of siRNA (nM) for luciferase gene knockdown are listed in the legend.

FIG. 20 depicts gel electrophoresis analysis of siRNAs (abbreviation below) released from hydrogels incubated at 2.5 mg/mL for control knockdown studies under reducing conditions (5 mM glutathione, GSH) in 10×PBS for 4 h at 37° C. lucAA¹: acrylamide non-degradable luciferase siRNA; NH₂²: native amine luciferase siRNA; lucPD³: degradable luciferase siRNA prodrug; crtlPD⁴: degradable control siRNA prodrug.

FIG. 21 depicts viability of HeLa/luc cells dosed with cationic hydrogels charged with different siRNA cargos. Cells were dosed with particles for 4 h followed by removal of particles and 48 h incubation in media.

FIGS. 22A & B: (a) Cellular uptake and (b) luciferase expression of HeLa/luc cells dosed with cationic hydrogels containing different siRNA cargos. Cells were dosed with particles for 4 h followed by removal of particles and 48 h incubation in media. Note that all hydrogels were thoroughly washed after fabrication to remove non-conjugated siRNA in the sol fraction.

FIG. 23 depicts an analysis of siRNA macromonomers by HPLC demonstrates that modifications of siRNA-NH₂ with acrylamide and disulfide precursors yield a single peak and increase retention time relative to unmodified siRNA-NH₂. Oligonucleotides were analyzed using 0.1 M triethylammonium acetate buffer (pH 7.0) and a gradient of 0 to 35% acetonitrile over 20 mM followed by a gradient to 100% acetonitrile over the next 15 mM HPLC runs were conducted at a flow rate of 0.2 mL/min using Zorbax Eclipse XDB-C18 column (4.6×150 mm, 5 μm, Agilent) and Agilent 1200 Series Multiple Wavelength Detector SL.

FIGS. 24A, B, C & D depict scanning microscopy image (SEM) of BSA nano and micro-sized particles fabricated using PRINT. A: 1 μm×1 μm cylinders, scale bar represents 10 μm, B: 3 μm×1 μm donut, scale bar represents 20 μm, C: 200 nm×200 nm cylinders, scale bar represents 4 μm, D: 3 μm×1 μm helicopters, scale bar represents 20 p.m.

FIGS. 25A, B & C depict BSA particle dissolution by microscopy image. (A) Particles transferred on to plasdone PET sheet (B) Particles with water added after 10 s, (C) Particles with water added after 5 min.

FIGS. 26A, B & C depict a synthetic route for several compounds described herein. A: DIC; B: OEDIC; C: tyramine-DIC.

FIGS. 27A, B & C depicts a GC-MS characterization of tyramine-DIC and tyramine-DSP after treatment with DTT, (a) standardard tyramine, (b) tyramine-DIC, (c) tyramine-DSP. The peak at 5.899 mM (m/z=152.0) in (b) and (c) represents oxidized DTT.

FIGS. 28A & B: (a) SEM image of BSA particles after incubation with water, particles were cross-linked at 4.4 mM of DIC, scale bar represents 10 μm. (b) Dissolution profile of crosslinked BSA particles in PBS containing 5 mM GSH (GSH) and PBS only (PBS), A: particles cross-linked at 4.4 mM of DIC, B: particles cross-linked at 6.6 mM of DIC, C: particles crosslinked at 9.9 mM of DIC, D: particles crosslinked at 4.4 mM of OEDIC. Squares with solid lines represent 5 mM GSH containing PBS and triangles with dotted lines represent PBS only. The error bars stand for the standard deviation calculated from three wells.

FIGS. 29A, B, C & D depict particle dissolution by microscopy image at 5-h time point. (A) Particles cross-linked with 4.4 mM of DIC, in PBS, (B) Particles cross-linked with 4.4 mM of DIC, in PBS containing 5 mM of GSH, (C) Particles cross-linked with 4.4 mM of OEDIC, in PBS (D) Particles cross-linked with 4.4 mM of OEDIC, in PBS containing 5 mM of GSH.

FIG. 30 depicts BSA activity measured by ELISA. Square represents BSA released from DIC-cross-linked PRINT particles, triangle represents untreated BSA, and tilted square represents heat-denatured BSA.

FIG. 31 depicts a PRINT process. BSA, lactose, glycerol and RNA replicon were mixed in water to create a solution. A film of this solution was drawn on a PET sheet with a myer rod. A solid film is generated after water is removed. A PRINT mold and the film are laminated together with the patterned side of the mold facing the film. The structure was then passed through a heated pressured nip and split. The PRINT mold with filled cavities is laminated onto a sacrificial adhesive layer on PET and passed through the nip again without splitting. After the particles cool down and solidify, mold and the PET were separated gently and particles are transferred to the sacrificial layer, which is then dissolved to release the particles.

FIGS. 32A & B: (a) Agarose gel of RNA replicon before and after particle crosslinking. 1: RNA ladder, 2: untreated RNA 200 ng, 3: untreated RNA 100 ng, 4: RNA replicon extracted out of blank BSA particles, 5: RNA replicon extracted out of BSA particles fabricated at 148° C., 6: RNA replicon extracted out of BSA particles fabricated at 60° C. (b) Relative fluorescence obtained from CAT ELISA. The absorbance from un-treated cells (cells only) were defined as 1. Error bars represent standard deviation calculated from four wells.

FIGS. 33A & B: (a) Scanning electron microscope (SEM) image of DIC-crosslinked particles containing CAT RNA replicon, image was taken after incubation with PBS, scale bar stands for 10 μm. (b) Agarose gel of RNA replicon before and after particle crosslinking: lane 1: RNA marker, 2: untreated RNA replicon 200 ng, 3: untreated RNA replicon 100 ng, 4: untreated RNA replicon 50 ng, 5: RNA replicon extracted out of BSA particles before crosslinking reaction, 6: RNA replicon extracted out of BSA particles after crosslinking reaction.

FIG. 34 depicts RNA replicon integrity after crosslinking reaction evaluated by CAT ELISA. The absorbance from un-treated cells (cells only) was defined as 1. Error bars stand for standard deviation calculated from four wells.

FIGS. 35A & B depict confocal microscopy of Vero cells dosed with PRINT protein particles, a) RNA replicon-containing BSA particles without TransIT, b) RNA replicon-containing BSA particles with TransIT, scale bar represents 30 μm.

FIG. 36 depicts CAT protein concentration generated from cells. Black: CAT RNA replicon standards delivered by TransIT, purple: blank particles (FIG. 37), orange: DIC-crosslinked BSA particles containing CAT RNA replicon, red: OEDIC-crosslinked particles containing CAT RNA replicon, blue: supernatant from particles incubated in PBS for 4 h at 37° C. Error bars represent standard deviation calculated from four wells.

FIG. 37 depicts CAT protein concentration generated from cells. Black: CAT RNA replicon standards delivered by TransIT, purple: blank particles, orange: DIC-crosslinked BSA particles containing CAT RNA replicon, red: OEDIC-crosslinked particles containing CAT RNA replicon, blue: supernatant from particles incubated in PBS for 4 h at 37° C. Error bars represent standard deviation calculated from four wells.

FIG. 38 depicts SEM image of OEDIC-crosslinked particles containing CAT RNA replicon, scale bar represents 5 μm.

FIGS. 39A, B & C: Confocal image of CAT protein. (A) 100 ng of CAT RNA replicon with TransIT, (B) blank particles, 2 μg/mL. (C) BSA particles containing CAT RNA replicon crosslinked with DIC, 2 μg/mL.

FIG. 40 depicts relative Luminesence generated by Luciferase encoded by PRINT particles. Blue: cells only, Black: CAT RNA replicon standards delivered by TransIT, purple: DIC-crosslinked BSA particles containing Luciferase RNA replicon, Error bars represent standard deviation calculated from four wells.

FIG. 41 depicts fluorescence generated from GFP encoded by RNA replicon delivered by PRINT particles. A) GFP RNA replicon standards delivered by TransIT, 100 ng/mL, B) GFP RNA replicon delivered by PRINT particles.

FIGS. 42A and B: Luciferase expression of viability of HeLa/luc cells dosed with luciferase and control sequences of native (siRNA-NH₂) and degradable siRNA prodrug (PD) complexed to Lipofectamine 2000™ and incubated for 48 h. Retention of siRNA activity after macromonomer synthesis was confirmed by evaluating transfection efficiency before and after siRNA derivatization.

### DETAILED DESCRIPTION

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims.

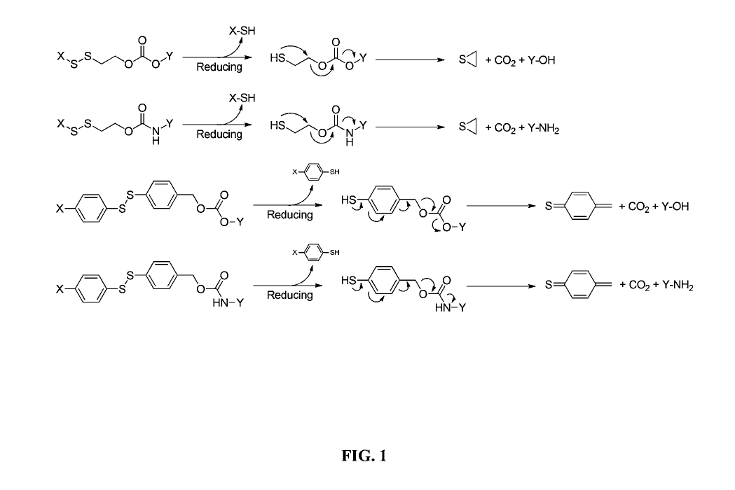

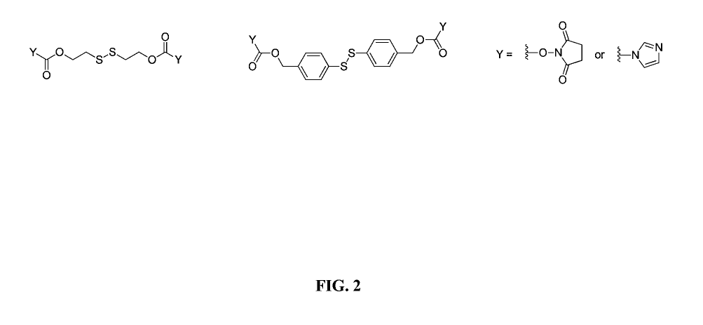

Disclosed herein are agents and delivery vehicles that have desirable properties. These properties are provided by either covalently linking a reversible linker(s) to the agent, the vehicle or both. The unique disulfide containing molecules disclosed herein can be referred to as conjugates, compounds or cross-linkers since in each instance the molecules are capable of linking desirable moieties, such as a drug, biomolecule, polymer or particle, through particular disulfide-containing chains. The cleavage of the disulfide bond results in a “traceless” linker since there are no molecular pendants remaining on the moieties themselves. This is particularly advantageous over linkers that leave pendant residues and is very useful increasing safety and efficacy of delivering therapeutics to the cell. While not being limited to a particular theory, FIG. 1 illustrates the traceless nature of the linkers due to the specific bonds and atoms from the position adjacent to the disulfide selection to the moiety. Cleavage of the disulfide bond, followed by further bond cleavages result in the native moiety, i.e., drug, biological, polymer, particle, etc., without any molecular pendants remaining on the moiety. Accordingly, the unique linkers described herein are completely reversible.

In some embodiments of the present invention, reversible moieties are attached to the surface of a particle to attach i) lipids, ii) water soluble polymers (e.g. poly(ethylene glycol)), and iii) reversible disulfide containing pro-drugs with the particle. Additionally, reversible disulfide chemistry is used to iv) introduce reversible disulfide linker-containing pro-drugs to the interior of a nanoparticle or liposome. According to such embodiments, the particle can facilitate delivery of a cargo, such as an agent or drug for example, in vivo safely and securely until a biological or chemical condition is reached which triggers reversing of the link chemistry and therefore release of the cargo. In further embodiments, the reversible disulfide chemistry is used to v) form a particle of a single chemical species by linking neighboring molecules together, such as for example, linking two or more of the same species of proteins together to form a particle of a given linked protein, wherein the reversibility of the present disulfide linker returns the protein to its native state following hydrolysis by leaving no residual chemical modification to the protein.

As disclosed herein, the reversible disulfide chemistry provides the ability to crosslink molecules, including the molecules that make up particles or hydrogels and the like. This crosslinking provides a useful way to entrap a cargo within the material of the particle or hydrogel. This can be accomplished without binding the cargo to the material of the particle or hydrogel. As described fully elsewhere herein, when the disulfide linker is contacted with or exposed to reducing conditions, the disulfide linkages can cleave. The disulfide linkers will degrade as described herein resulting in loss of at least some cross-linking of the material. Once a molecule of the particle or hydrogel is no longer cross-linked, the cargo entrapped by the material can then release or diffuse from the particle or hydrogel. Accordingly, the disulfide chemistry disclosed herein is beneficial to targeted delivery of the cargo to areas having the conditions that will cleave the disulfide bond, such as the cytoplasm of cells.

In alternative embodiments the reversible linker includes a trityl moiety, an ester moiety, or a CDM (carboxylated dimethyl maleic acid) moieties. As will be appreciated by one of skill in the art, the alternative linker moieties can used in place of the disulfide linker described herein. For convenience of drafting, the specification will be primarily focused on disulfide linkers but it should be appreciated that the alternative moieties can be substituted therewith where applicable.

The term “reversible” means that the particle and/or compostion or component thereof covalently linked to a reversible disulfide linker has the same structure upon hydrolysis of the disulfide linker as before conjugation.

The term “therapeutic,” “therapeutic agent,” “active,” “active agent,” “active pharmaceutical agent,” “active drug” or “drug” as used herein means any active pharmaceutical ingredient (“API”), including its pharmaceutically acceptable salts (e.g. the hydrochloride salts, the hydrobromide salts, the hydroiodide salts, and the saccharinate salts), as well as in the anhydrous, hydrated, and solvated forms, in the form of prodrugs, and in the individually optically active enantiomers of the API as well as polymorphs of the API. Therapeutic agents include pharmaceutical, chemical or biological agents. Additionally, pharmaceutical, chemical or biological agents can include any agent that has a desired property or affect whether it is a therapeutic agent. For example, agents also include diagnostic agents, biocides and the like. The reversible disulfide-containing agent, etc. can also be referred to as a conjugate. Preferred biological agents include proteins or fragments thereof.

As used herein “component” refers to a part of a vehicle. Accordingly, the component can be the reversible disulfide-containing agent, drug, conjugate, etc. The component can be covalently linked to the vehicle or contained inside the vehicle, e.g. in a lumen or simply within a substance that makes up the bulk of a particle.

As used herein the term “mammal” refers to humans as well as all other mammalian animals. As used herein, the term “mammal” includes a “subject” or “patient” and refers to a warm blooded animal.

As used herein, the terms “cancer” and “cancerous” refer to or describe the physiological condition in mammals that is typically characterized by unregulated cell growth. Examples of cancer include, but are not limited to, melanoma, carcinoma, lymphoma, blastoma, sarcoma, and leukemia or lymphoid malignancies. More particular examples of cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

As used herein, the term “therapeutically effective” and “effective amount,” is defined as the amount of the pharmaceutical composition that produces at least some effect in treating a disease or a condition. For example, in a combination according to the invention, an effective amount is the amount required to inhibit the growth of cells of a neoplasm in vivo. The effective amount of active compound(s) used to practice the present invention for therapeutic treatment of neoplasms (e.g., cancer) varies depending upon the manner of administration, the age, body weight, and general health of the subject. It is within the skill in the art for an attending physician or veterinarian to determine the appropriate amount and dosage regimen. Such amounts may be referred to as “effective” amounts.

An “active agent moiety” in reference to a prodrug conjugate of the invention, refers to the portion or residue of the unmodified parent active agent up to the covalent linkage resulting from covalent attachment of the drug (or an activated or chemically modified form thereof) to a polymer of the invention. Upon hydrolysis of the linkage between the active agent moiety and the multi-armed polymer, the active agent per se is released.

As used herein, the term “ligand” refers to a molecule that can be used to target a desired area or tissue. The ligand will have an affinity for the desired tissue based on intrinsic properties of the ligand and the target.

Agents, e.g., pharmaceutical, chemical and biological compounds, which contain a reversible disulfide linker could also contain a reversible disulfide bond in their native structure. However, the reversible disulfide linker is in addition to any native disulfide bond of the agent. Examples of such agents having a native disulfide bond include certain proteins, antibodies, and other agents as will be appreciated by one of ordinary skill in the art.

In one embodiment, a therapeutic agent, such as a pharmaceutical, chemical or biological agent is covalently linked to a reversible disulfide linker. In this embodiment, the resulting compound can act as a pro-drug of the agent. Methods of preparing such reversible disulfide containing compounds are described herein. In an embodiment, the present subject matter is directed to a compound of the formula:

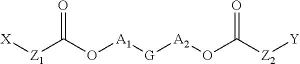

wherein, X and Y are each independently selected from the group consisting of a polymerizable moiety and a leaving group; G is O or —S—S—; A₁ and A₂ are each —C(R^{a}R^{b})—C(R^{c}R^{d})—, wherein in each instance, R^{a}, R^{b}, R^{c} and R^{d} are each independently selected from the group consisting of hydrogen, C₁₋₆ alkyl and hydroxyl; and

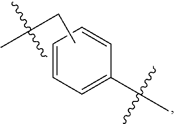

wherein G is adjacent to the ring; and Z₁ and Z₂ are each independently selected from the group consisting of NH, O or S.

In this embodiment, useful polymerizable moieties include —CH═CH₂, —C(CH₃)═CH₂, —CH═CH—O—CH═CH₂, vinyl ester, N-vinyl carbazole and N-vinyl pyrrolidone.

In this embodiment, useful leaving groups are selected from the group consisting of triflate, tosyl, Cl,

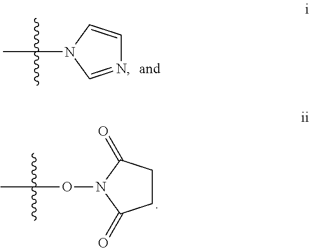 [SMILES: *n1ccnc1 ; *ON1C(=O)CCC1=O]

Preferably, in each instance, R^{a}, R^{b}, R^{c} and R^{d} are each hydrogen.

Preferred compounds are of the formula:

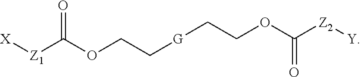

In compounds where G is O, the ether moiety results in a bond that does not cleave under reducing conditions. While this may be a useful characteristic, it results in a molecule that is a non-reversible conjugate. Therefore, in all embodiments, it is preferred that G is —S—S—, wherein said compound is of the formula:

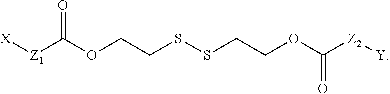

The disulfide linkage provides a bond that can be cleaved under reducing conditions. As used herein, reducing conditions describe conditions under which the disulfide bond will cleave. This can occur when there is a presence of a reducing agent such as glutathione or mercaptoethanol. In the cytoplasm of a cell the there is the glutathione system which is a compilation of reductive enzymes and glutathione.

Preferred compounds include those where at least one of Z₁ and Z₂ is O or N. More preferred compounds are those where Z₁ and Z₂ are both O or N.

Preferred compounds include those having one of the following structures:

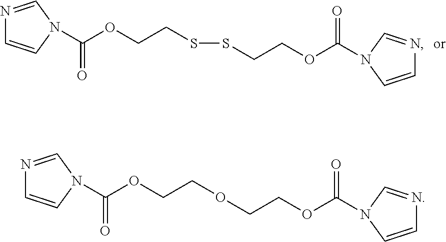 [SMILES: O=C(OCCSSCCOC(=O)n1ccnc1)n1ccnc1 ; O=C(OCCOCCOC(=O)n1ccnc1)n1ccnc1]

In an embodiment, the present subject matter is directed to a conjugate of the formula:

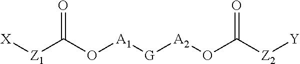

wherein, X is a drug, a biomolecule, a polymer or a particle; Y is selected from the group consisting of a polymerizable moiety, a leaving group, a drug, a biomolecule, a polymer and a particle; G is —S—S—; A₁ and A₂ are each independently selected from the group consisting of —C(R^{a}R^{b})—C(R^{c}R^{d})—, wherein R^{a}, R^{b}, R^{c} and R^{d} are each independently selected from the group consisting of hydrogen, C₁₋₆ alkyl and hydroxyl; and

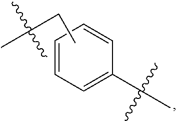

wherein G is adjacent to the ring; and Z₁ and Z₂ are each independently selected from the group consisting of NH, O or S.

In this embodiment, the drug, biomolecule, polymer or particle is covalently attached through the O, N or S that is distal from the disulfide or ether linkage, represented herein generically as G, in the conjugate. Accordingly, any drugs, biomolecules, polymers or particles that have a nucleophilic group capable of covalently binding to O, N or S are suitable molecules for incorporation into the conjugate. These nucleophile groups include, but are not limited to, hydroxyls, amines, thiols. One of skill in this art would readily determine such molecules. Examples of such molecules having nucleophile groups are disclosed elsewhere herein.

The conjugated drug, biomolecule, polymer or particle can also be referred to as a residue of the native drug, biomolecule, polymer or particle. As shown herein, the conjugated drug, biomolecule, polymer or particle can be returned to its native form under reducing conditions when the disulfide linker cleaves. The result is a virtually traceless cross-linker that upon cleavage leaves no chemical residue or pendant on the native drug, biomolecule, polymer or particle. The preferred compounds and conjugates described herein are therefore substantially completely reversible linkers.

Preferred conjugates include those where X is a biomolecule selected from the group consisting of a lipid, a protein, an oligonucleotides, siRNA, RNA replicon, cDNA, nucleic acids, morpholinos, peptide nucleic acids, polysaccharides, sugars and enzymes.

Preferred conjugates are those where in each instance, R^{a}, R^{b}, R^{c} and R^{d} are each hydrogen.

Preferred conjugates include those where G is —S—S—, wherein said conjugate is of the formula:

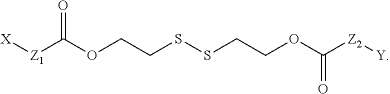

Preferred conjugates include those where Y is a drug, a biomolecule, a polymer or a particle.

When Y is a biomolecule, the biomolecule can be selected from the group consisting of a lipid, a protein, an oligonucleotides, siRNA, RNA replicon, cDNA, nucleic acids, morpholinos, peptide nucleic acids, polysaccharides, sugars and enzymes.

When Y is a drug, which is also referred to as an agent. Such drugs are described elsewhere herein.

When Y is a polymer, the polymer can be selected from the group consisting of PEG.

When Y is a polymerizable moiety, it can be selected from the group consisting of —CH═CH₂, —C(CH₃)═CH₂, —CH═CH—O—CH═CH₂, vinyl ester, N-vinyl carbazole and N-vinyl pyrrolidone.

When Y is a leaving group, it can be selected from the group consisting of group selected from the group consisting of triflate, tosyl, Cl,

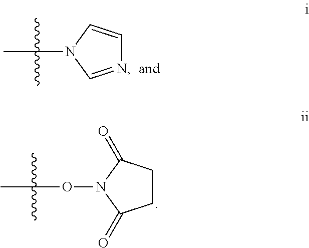 [SMILES: *n1ccnc1 ; *ON1C(=O)CCC1=O]

In another embodiment, the subject matter described herein is directed to a conjugate of one of the following formulae:

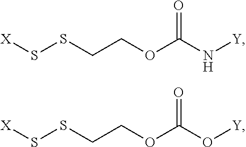

wherein X is

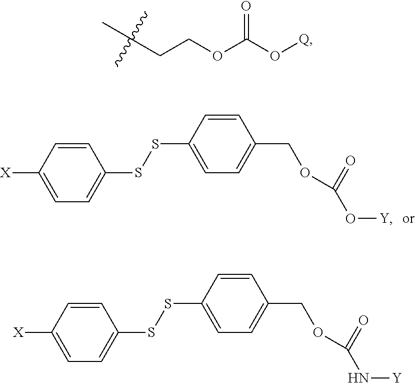

wherein X is

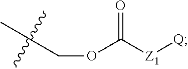

wherein, Z₁ is O, N or S and Q is a polymerizable moiety or a leaving group; and Y is a drug, a biomolecule, a polymer or a particle.

When Q is a polymerizable moiety, it can be selected from the group consisting of —CH═CH₂, —C(CH₃)═CH₂, —CH═CH—O—CH═CH₂, vinyl ester, N-vinyl carbazole and N-vinyl pyrrolidone.

When Q is a leaving group, it can be selected from the group consisting of triflate, tosyl, Cl,

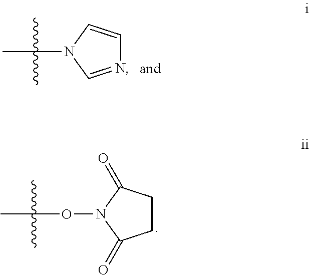 [SMILES: *n1ccnc1 ; *ON1C(=O)CCC1=O]

In an embodiment, the present subject matter is directed to a method of preparing a targeted delivery system comprising: covalently linking a compound, conjugate or crosslinker as described herein to a drug, biomolecule, polymer or particle, wherein the resulting bound drug, biomolecule, polymer or particle is capable of targeting specific areas, tissues, cells, etc.

In an embodiment, the present subject matter is directed to a method of preparing a compound, conjugate or crosslinker as described herein.

Additional specific embodiments of the present disclosure include:
1. A delivery vehicle, wherein the vehicle comprises a component and a reversible disulfide linker either covalently bound to the vehicle or contained within the vehicle.
2. The delivery vehicle of embodiment 1, wherein the reversible disulfide linker is present on an exterior surface of the vehicle.
3. The delivery vehicle of embodiment 2, wherein the reversible disulfide linker is covalently linked to the exterior surface.
4. The delivery vehicle of embodiment 1, wherein the reversible disulfide linker is present in the interior of the vehicle.
5. The delivery vehicle of embodiment 4, wherein the reversible disulfide linker is covalently linked to the interior of said vehicle.
6. The delivery vehicle of embodiment 1, wherein the component comprises a lipid, polymer, ligand, tracer, chemical agent, pharmaceutical agent or biological agent.
7. The delivery vehicle of embodiment 6, wherein the polymer is a water soluble polymer.
8. The delivery vehicle of embodiment 7, wherein the polymer is a PEG.
9. The delivery vehicle of embodiment 1, wherein the vehicle is selected from the group consisting of a liposome, particle, microparticle and nanoparticle.
10. The delivery vehicle of embodiment 9, wherein the vehicle is a liposome.
11. The delivery vehicle of embodiment 9, wherein the vehicle is a nanoparticle.
12. A compound having a reversible disulfide linker covalently bound to a pharmaceutical, chemical or biological agent.
13. The compound of embodiment 12, wherein the reversible disulfide linker is covalently bound to a pharmaceutical agent.
14. The compound of embodiment 13, wherein the pharmaceutical agent is selected from the group consisting of analgesics, anti-cancer agents, anti-inflammatory agents, antihelminthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-blockers, cardiac inotropic agents, corticosteroids, diuretics, anti-parkinsonian agents, gastro-intestinal agents, histamine receptor antagonists, keratolyptics, lipid regulating agents, anti-anginal agents, Cox-2 inhibitors, leukotriene inhibitors, macrolides, muscle relaxants, nutritional agents, opioid analgesics, protease inhibitors, sex hormones, stimulants, muscle relaxants, anti-osteoporosis agents, anti-obesity agents, cognition enhancers, anti-urinary incontinence agents, anti-benign prostate hypertrophy agents, essential fatty acids, non-essential fatty acids, and mixtures thereof.
15. The compound of embodiment 14, wherein the pharmaceutical agent is an anti-cancer agent.
16. The compound of embodiment 12, wherein the pharmaceutical or biological agent is selected from quinoline alkaloids, taxanes, anthracyclines, nucleosides, kinase inhibitors, tyrosine kinase inhibitors, antifolates, proteins and nucleic acids.
17. The compound of embodiment 12, wherein the pharmaceutical or biological agent is selected from the group consisting of Camptothecin, Topotecan, Irinotecan, SN-38, Paclitaxel, Docetaxel, Daunorubicin, Doxorubicin, Epirubicin, Idarubicin Gemcitabine, Cytarabine, Brefeldin-A Imatinib, Gefitinib, Lapatinib, Sunitinib, Methotrexate, Folinic Acid, Efflux Inhibitors, ATP-Binding Inhibitors, Cytochrome-C, Ovalbumin, siRNA Anti-Luciferase, siRNA Androgen Receptor and RNA Replicon.
18. The compound of embodiment 12, wherein reversible disulfide linker is covalently bound to a biological agent
19. The compound of embodiment 18, wherein the biological agent is DNA, RNA, siRNA, shRNA, miRNA, RNA replicon, cDNA, proteins or immunoglobulins.
20. The compound of embodiment 12, wherein the reversible disulfide linker is covalently bound to a chemical agent.
21. The compound of embodiment 20, wherein the chemical agent is a pesticide, fungicide, insecticide, herbicide or biocide.
22. The compound of embodiment 12, wherein the reversible disulfide linker is further covalently bound to a lipid, polymer, ligand or tracer.
23. A method of treating a mammal, comprising administering a compound of claim 12, wherein the compound comprises a pharmaceutical or biological agent.
24. A method of treating a mammal, comprising administering a delivery vehicle of embodiment 1.
25. A method of modifying a property of an agent, comprising preparing a reversible disulfide linker covalently linked to said agent, wherein said agent is a pharmaceutical, chemical or biological agent.
26. The method of embodiment 25, wherein said reversible disulfide linker is further covalently bound to a lipid, polymer, ligand or tracer.
27. A method of modifying a property of a first agent comprising allowing the agent to contact a reversible disulfide linker wherein a reversible disulfide containing agent is prepared, wherein a property of said first agent is modified.
28. The method of embodiment 27, wherein said property is solubility in an aqueous milieu.
29. The method of embodiment 27, wherein said property is stability under physiological conditions other than the target tissue.
30. The method of embodiment 27, wherein said property is hydrophobicity.
31. A delivery vehicle, comprising a particle, wherein the particle comprises a composition, wherein the composition comprises a component, wherein the component is covalently linked to a reversible disulfide linker.
32. The delivery vehicle of embodiment 31, wherein said component is an agent.
33. The delivery vehicle of embodiment 32, wherein said agent is a pro-drug.
34. The delivery vehicle of embodiment of claim 31, wherein the reversible disulfide linker is a residue of a linker selected from the group consisting of Formulae I, II, III and IV, and compounds 1-9.
35. The delivery vehicle of embodiment 31, further comprising a second component.
36. The delivery vehicle of embodiment 34, wherein the first and second components are independently selected from a pharmaceutical, chemical or biological agent, a lipid, a polysaccharide, a protein and a polymer.
37. The delivery vehicle of embodiment 36, wherein said biological agent is a protein.
38. The delivery vehicle of embodiment 31, wherein said vehicle is a nanoparticle or a liposome.
39. The delivery vehicle of embodiment 31, of claim 1, further comprising a second component in the composition, wherein the two components are selected from a protein and a polysaccharide.
40. The delivery vehicle of embodiment 31, further comprising a second component in the composition wherein the two components are selected from a protein and a lipid.
41. The delivery vehicle of embodiment 31, further comprising a second component in the composition wherein the two components are selected from a first protein and a second protein.
42. The delivery vehicle of embodiment 31, further comprising a second component in the composition wherein the two components are selected from a first polysaccharide and a second polysaccharide.
43. The delivery vehicle of claim 1, further comprising a second component in the composition wherein the two components are selected from a first lipid and a second lipid.
44. A drug delivery particle comprising, a particle having a component linked to a surface of the particle wherein the link comprises a reversible disulfide linker.
45. The drug delivery particle of embodiment 44, wherein the reversible disulfide linker is a residue of a linker selected from the group consisting of Formulae I, II, III and IV, and compounds 1-9.
46. The drug delivery particle of claim 44, wherein the particle comprises a protein and the component comprises a polysaccharide.
47. The drug delivery particle of embodiment 44, wherein the particle comprises a protein and the component comprises a lipid.
48. The drug delivery particle of embodiment 44, wherein the particle comprises a first protein and the component comprises a second protein.
49. The drug delivery particle of embodiment 44, wherein the particle comprises a first polysaccharide and the component comprises a second polysaccharide.
50. The drug delivery particle of embodiment 44, wherein the particle comprises a first lipid and the component comprises a second lipid.
51. A drug delivery particle comprising, a particle comprised of a polymeric hydrogel and a siRNA coupled with the polymer through a reversible disulfide linker.
52. The particle of embodiment 51, wherein the polymeric hydrogel is PEG.
53. A composition comprising a component covalently linked with a residue of a linker selected from the group consisting of Formulae I, II, III and IV, and compounds 1-9.
54. The composition of embodiment 54, wherein said component is agent is selected from a pharmaceutical, chemical or biological agent, a lipid, a polysaccharide, a protein and a polymer.
55. The composition of embodiment 54, wherein said agent is a drug.
56. A method of preparing a drug delivery particle by combining an agent and a reversible disulfide linker in a mold, whereby the agent is covalently linked to the linker.
57. The method of embodiment 56, further comprising allowing the covalently linked agent to further covalently bind to the particle.
58. A method of delivering the drug delivery particle of embodiment 56, comprising administering the particle to a subject.
59. The compound of embodiment 12, wherein the linker is a residue of a linker selected from structures 1-9.
60. The compound of embodiment 12 selected from the group consisting of structures 10-24.
61. The delivery vehicle of embodiment 6, wherein the component is a conjugate covalently linked to the linker.
62. The delivery vehicle of embodiment 61, wherein the conjugate is further covalently linked to the vehicle.
63. A drug delivery particle, comprising; a particle having a composition, wherein the composition comprises a component and a reversible disulfide cross linker wherein following degradation of the reversible disulfide cross linker no chemical pendant groups remain associated with the component.
64. The particle of embodiment 63, wherein the reversible disulfide cross linker is selected from one of the following structures.
65. The particle of embodiment 63, further comprising a second component in the composition, wherein the two components are selected from a protein and a polysaccharide.
66. The particle of embodiment 63, further comprising a second component in the composition wherein the two components are selected from a protein and a lipid.
67. The particle of embodiment 63, further comprising a second component in the composition wherein the two components are selected from a first protein and a second protein.
68. The particle of embodiment 63, further comprising a second component in the composition wherein the two components are selected from a first polysaccharide and a second polysaccharide.
69. The particle of embodiment 63, further comprising a second component in the composition wherein the two components are selected from a first lipid and a second lipid.
70. A drug delivery particle, comprising; a particle having an agent linked to a surface of the particle wherein the link comprises a reversible disulfide cross linker.
71. The particle of embodiment 70, wherein the reversible disulfide cross linker is selected from Formulae I, II, III and IV.
72. The particle of embodiment 70, wherein the particle comprises a protein and the agent comprises a polysaccharide reversibly linked with the reversible disulfide cross linker.
73. The particle of embodiment 70, wherein the particle comprises a protein and the agent comprises a lipid reversibly linked with the reversible disulfide cross linker.
74. The particle of embodiment 70, wherein the particle comprises a first protein and the agent comprises a second protein reversibly linked with the reversible disulfide cross linker.
75. The particle of embodiment 70, wherein the particle comprises a first polysaccharide and the agent comprises a second polysaccharide reversibly linked with the reversible disulfide cross linker.
76. The particle of embodiment 70, wherein the particle comprises a first lipid and the agent comprises a second lipid reversibly linked with the reversible disulfide cross linker.
77. A drug delivery particle, comprising; a particle comprised of a polymeric hydrogel and an siRNA coupled with the polymer through a reversible disulfide cross linker.
78. The particle of embodiment 77, wherein the polymeric hydrogel is PEG.
79. A drug composition, comprising: a macromolecule reversibly cross linked with an agent through a reversible disulfide cross linker.
80. A method of delivering a drug, comprising; fabricating a drug delivery particle by combining an agent and a reversible disulfide cross linker in a mold and activating cross linking of the reversible disulfide cross linker to couple the agent.
81. The method of embodiment 80, wherein the reversible disulfide cross linker leaves no chemical pendant groups on the agent after the reversible disulfide cross linker is reduced such that the agent is returned to a native state.

The reversible disulfide linkers described herein can take advantage of the redox potential difference between intracellular and extracellular environment, where glutathione (GSH) concentration differs by as much as 1000× or more. A nanoparticle containing a reversible disulfide linker as described herein once at the target, for example, in the cytosol, degrades and slowly releases the cargo upon interactions with GSH. Since the reversible disulfide linker degrades, the native protein, molecule, agents, etc. are delivered to the target tissue. The reaction cascade can be initiated by GSH as a reducing agent and the formed free thiol as a nucleophile to intramolecularly cyclize to release the free amine or alcohol. The synthesis of the crosslinker is straightforward as described herein. Similar chemistry can also apply to alcohols to form carbonate.

In an embodiment, the present subject matter is directed to reversible disulfide linkers of the following general formulae:

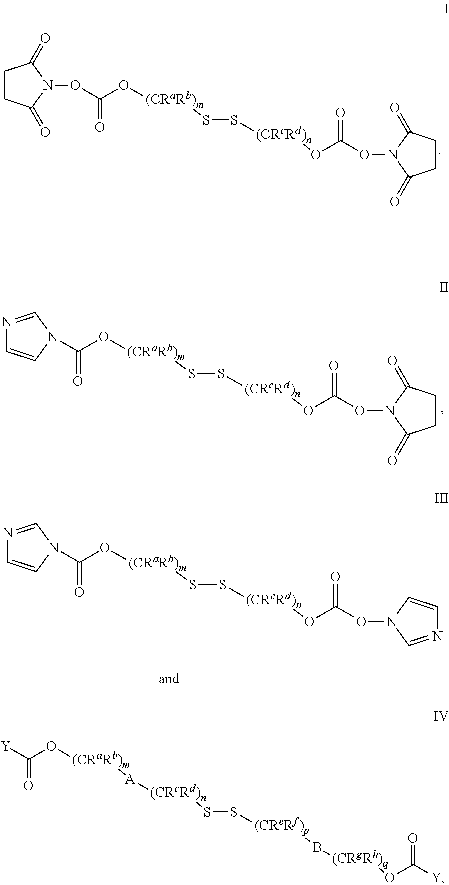

wherein,

R^{a}, R^{b}, R^{e}, R^{d}, R^{e}, R^{f}, R^{g} and R^{h} are each independently selected from the group consisting of hydrogen, C₁₋₆ alkyl and hydroxyl,

m, n, p and q are independently of each other an integer from zero to four,

A is a 5- to 10-member, optionally substituted aryl or heteroaryl ring,

B is a 5- to 10-member, optionally substituted aryl or heteroaryl ring,

wherein A and B can de the same or different, and

Y is a leaving group. Leaving groups are well known in the art. Exemplified leaving groups include triflate, tosyl, Cl, as well as a moiety selected from the group consisting of i and ii:

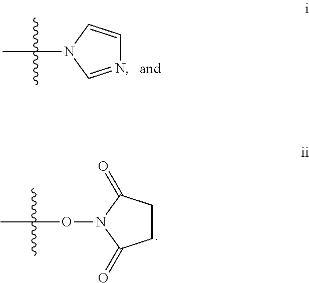 [SMILES: *n1ccnc1 ; *ON1C(=O)CCC1=O]

When m, n, o or p are two or four, the resulting —(CRR)—(CRR)— or —(CRR)—(CRR)—(CRR)—(CRR)— can be unsaturated, such as —(CR═CR)— or —(CR═CR—CR═CR)—.

The term “aryl” as employed herein by itself or as part of another group refers to monocyclic or bicyclic aromatic groups containing from 6 to 12 carbons in the ring portion, preferably 6-10 carbons in the ring portion, such as phenyl, naphthyl or tetrahydronaphthyl. The term “heteroaryl” as employed herein refers to groups having 5 to 14 ring atoms; 6, 10 or 14 n electrons shared in a cyclic array; and containing carbon atoms and 1, 2, 3 or 4 oxygen, nitrogen or sulfur heteroatoms (where examples of heteroaryl groups are: thienyl, benzo[b]thienyl, naphtho[2,3-b]thienyl, thianthrenyl, furyl, pyranyl, isobenzofuranyl, benzoxazolyl, 2H-pyrrolyl, pyrrolyl, imidazolyl, pyrazolyl, pyridinyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, phenazinyl, isothiazolyl, phenothiazinyl, isoxazolyl and furazanyl groups).

The aryl or heteroaryl ring may be optionally substituted with alkyl, alkoxy, halogen, amine, monoalkylamine, dialkylamine and hydroxyl.

The term “alkyl” as employed herein by itself or as part of another group refers to both straight and branched chain radicals of up to 8 carbons, preferably 6 carbons, more preferably 4 carbons, such as methyl, ethyl, propyl, isopropyl, butyl, t-butyl, and isobutyl.

The term “alkoxy” is used herein to mean a straight or branched chain alkyl radical, as defined above, unless the chain length is limited thereto, bonded to an oxygen atom, including, but not limited to, methoxy, ethoxy, n-propoxy, isopropoxy, and the like. Preferably the alkoxy chain is 1 to 6 carbon atoms in length, more preferably 1-4 carbon atoms in length.

The term “monoalkylamine” as employed herein by itself or as part of another group refers to an amino group which is substituted with one alkyl group as defined above.

The term “dialkylamine” as employed herein by itself or as part of another group refers to an amino group which is substituted with two alkyl groups as defined above.

The term “halogen” employed herein by itself or as part of another group refers to chlorine, bromine, fluorine or iodine.

In an embodiment, the reversible disulfide linker is polymerizable. In this embodiment, one, but not both, of Y is:

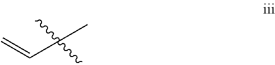 [SMILES: *C=C]

Specific compounds described herein include:

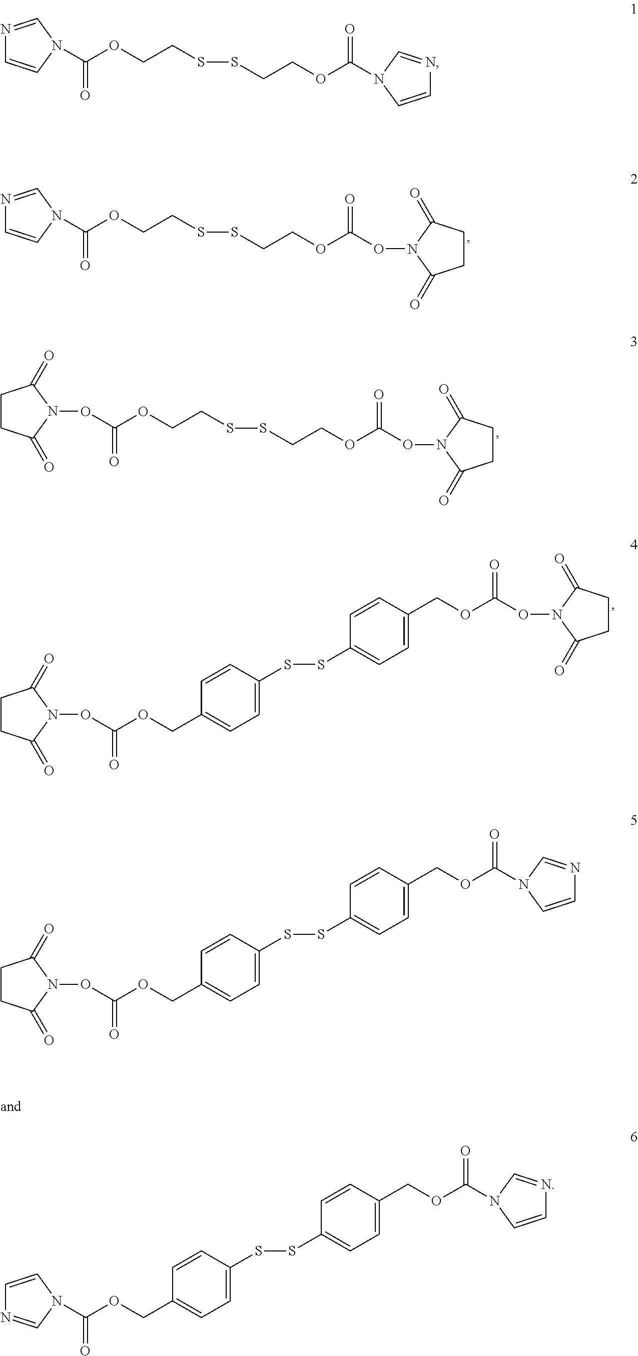 [SMILES: O=C(OCCSSCCOC(=O)n1ccnc1)n1ccnc1 ; O=C(OCCSSCCOC(=O)n1ccnc1)ON1C(=O)CCC1=O ; O=C(OCCSSCCOC(=O)ON1C(=O)CCC1=O)ON1C(=O)CCC1=O ; O=C(OCc1ccc(SSc2ccc(COC(=O)ON3C(=O)CCC3=O)cc2)cc1)ON1C(=O)CCC1=O ; O=C(OCc1ccc(SSc2ccc(COC(=O)n3ccnc3)cc2)cc1)ON1C(=O)CCC1=O ; O=C(OCc1ccc(SSc2ccc(COC(=O)n3ccnc3)cc2)cc1)n1ccnc1]

Polymerizable linkers are also provided herein, wherein one end of the linker contains a polymerizable moiety, such as an acrylic moiety. Specific examples include:

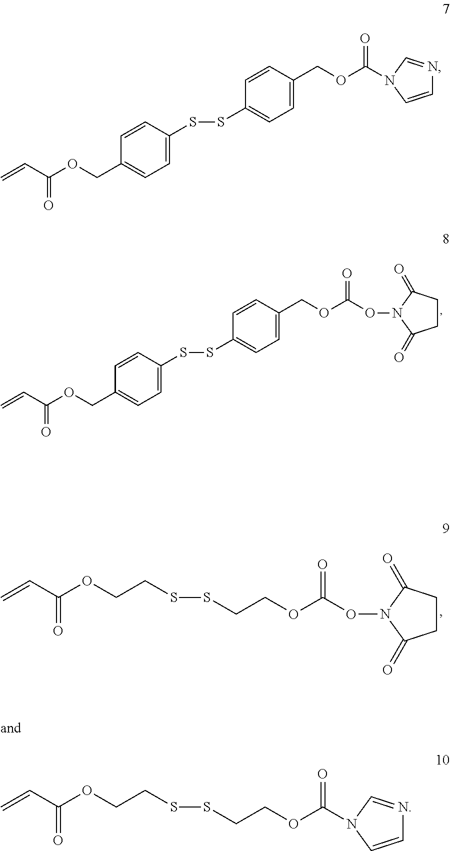 [SMILES: C=CC(=O)OCc1ccc(SSc2ccc(COC(=O)n3ccnc3)cc2)cc1 ; C=CC(=O)OCc1ccc(SSc2ccc(COC(=O)ON3C(=O)CCC3=O)cc2)cc1 ; C=CC(=O)OCCSSCCOC(=O)ON1C(=O)CCC1=O ; C=CC(=O)OCCSSCCOC(=O)n1ccnc1]

In an embodiment, the subject matter disclosed herein is directed to compounds having an agent covalently linked to a reversible disulfide linker. Examples include:

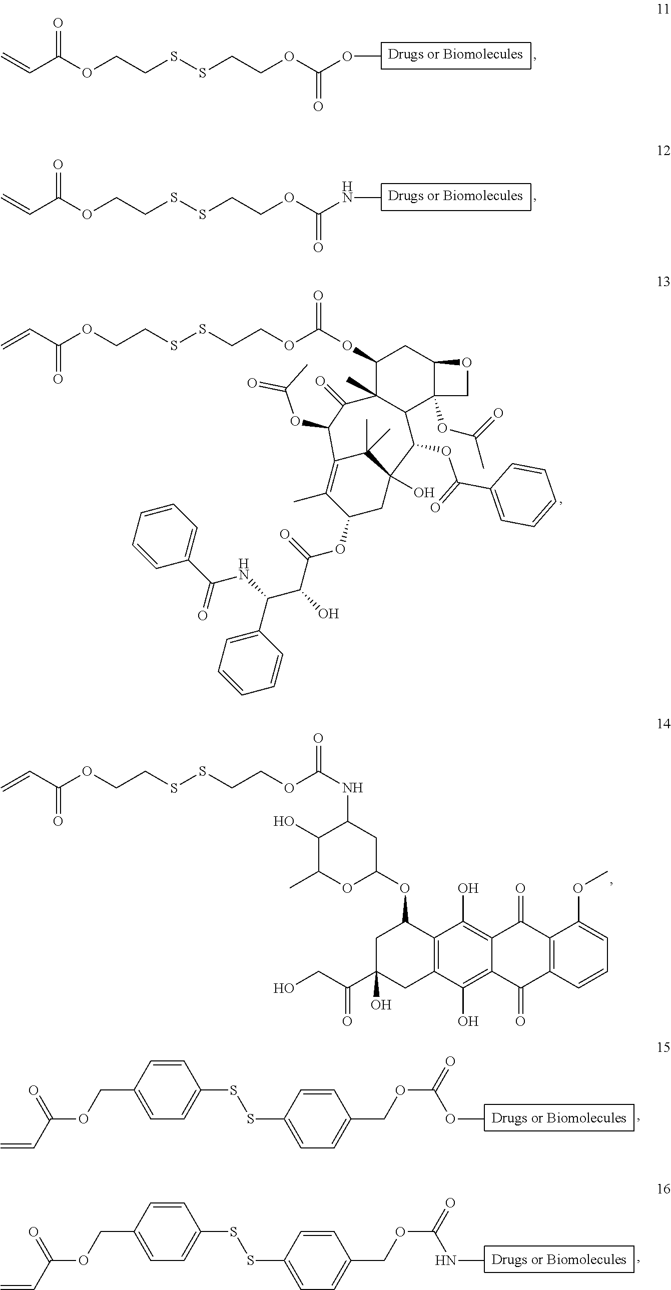

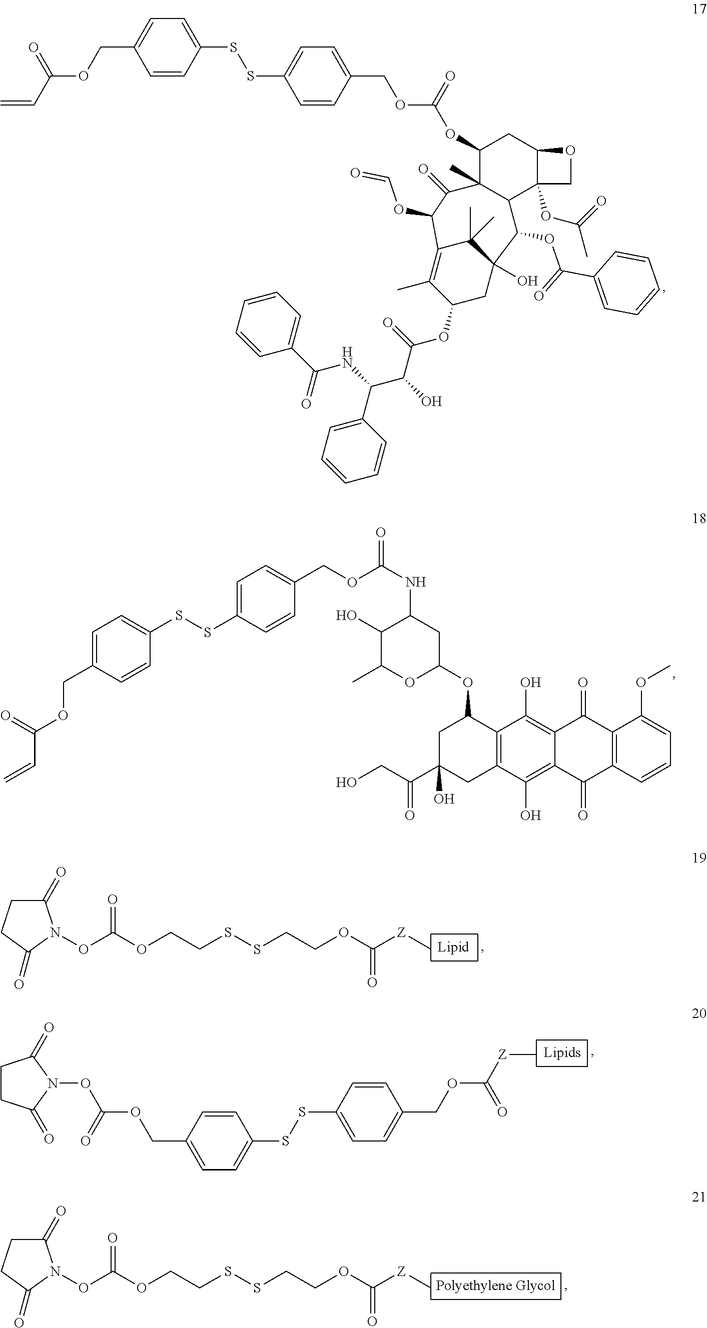

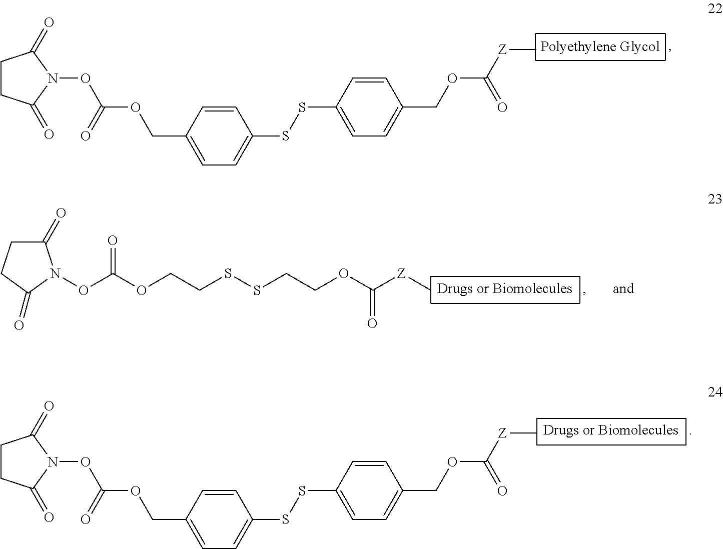

wherein Z is NH, O or S.

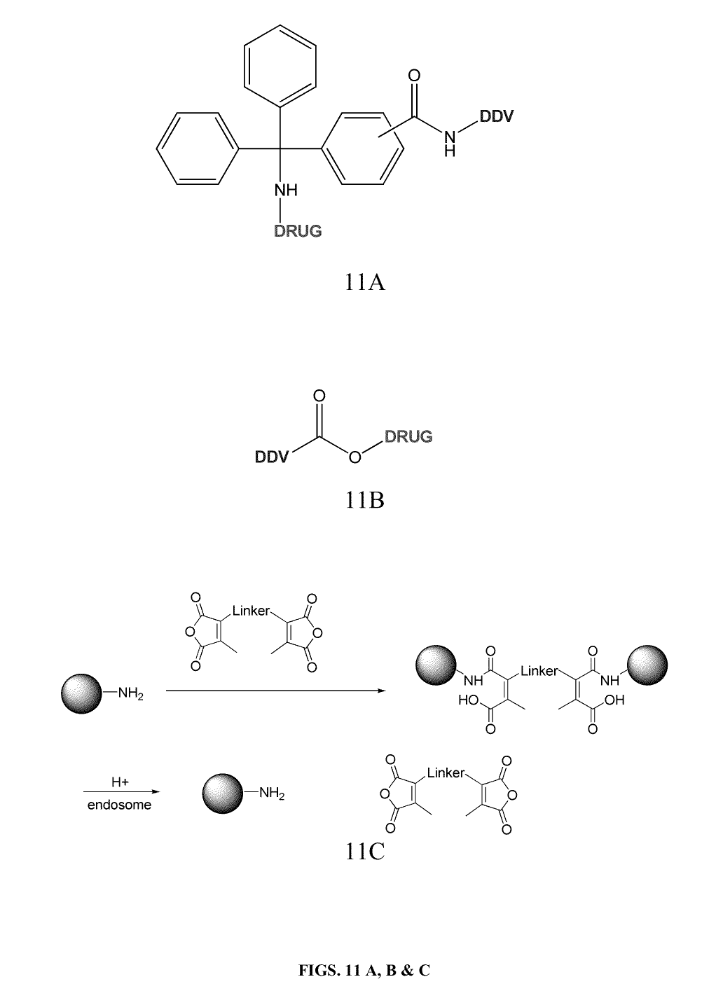

In addition to the reversible disulfide linkers disclosed herein, other reversible linkers are contemplated so long as upon degradation of the linker, no remnants of the linker remain on the particles and/or composition or component thereof. Other suitable linkers include linkers based on trityl, ester and CDM carboxylated dimethyl maleic acid chemistries. Examples are shown in FIG. 11. The preferred linkers are the reversible disulfide linkers disclosed herein.

Preferred pharmaceutical agents that can be modified with a reversible disulfide linker include Camptothecin, Topotecan, Irinotecan, SN-38, Paclitaxel, Docetaxel Daunorubicin, Doxorubicin, Epirubicin, Idarubicin Gemcitabine, Cytarabine Brefeldin-A Imatinib, Gefitinib, Lapatinib, Sunitinib Methotrexate, Folinic Acid Efflux Inhibitors, ATP-Binding Inhibitors Cytochrome-C, siRNA, e.g., Ovalbumin siRNA Anti-Luciferase, siRNA Androgen Receptor, and RNA Replicon.

Other agents include Busulfan, Chlorambucil, Cyclophosphamide, melphalan, Carmustine, Lomustine, Cladribine, Cytarabine (Cytosine Arabinoside), Floxuridine (FUDR, 5-Fluorodeoxyuridine), Fludarabine, 5-Fluorouracil (5FU), Hydroxyurea, 6-Mercaptopurine (6 MP), Methotrexate (Amethopterin), 6-Thioguanine, Pentostatin, Pibobroman, Tegafur, Trimetrexate, Glucuronate, 5-Fluorouracil (5-FU), Pemetrexed, Antitumor antibiotics including Aclarubicin, Bleomycin, Dactinomycin (Actinomycin D), Mitomycin C, Mitoxantrone, Plicamycin (Mithramycin), Mitotic inhibitors include plant alkaloids and other natural agents that can inhibit either protein synthesis required for cell division or mitosis, Docetaxel, Vinblastine sulfate, Vincristine, Etoposide (VP16), Carboplatin, cisplatin and oxaliplatin.

In further embodiments the subject matter disclosed herein can be utilized with the particles and compositions disclosed in the following co-pending patent application publications, each of which are incorporated herein by reference in their entirety: US 2009/0028910; US 2009/0061152; WO 2007/024323; US 2009/0220789; US 2007/0264481; US 2010/0028994; US 2010/0196277; WO 2008/106503; US 2010/0151031; WO 2008/100304; WO 2009/041652; PCT/US2010/041797; US 2008/0181958; WO 2009/111588; and WO 2009/132206.

### RNA Delivery

A critical need still remains for effective delivery of RNA interference (RNAi) therapeutics to target tissues and cells. Self-assembled lipid- and polymer-based systems have been most extensively explored for transfection of small interfering RNA (siRNA) in liver and cancer therapies. Safety and compatibility of materials implemented in delivery systems must be ensured to maximize therapeutic indices. Hydrogel nanoparticles of defined dimensions and compositions, prepared via a particle molding process that is a unique off-shoot of soft lithography known as PRINT (Particle Replication in Non-wetting Templates), were explored in these studies as delivery vectors. Initially, siRNA was encapsulated in particles through electrostatic association and physical entrapment. Dose-dependent gene silencing was elicited by PEGylated hydrogels at low siRNA doses without cytotoxicity. To prevent disassociation of cargo from particles after systemic administration or during post-fabrication processing for surface functionalization, a polymerizable siRNA pro-drug conjugate with a degradable, disulfide linkage was prepared. Triggered release of siRNA from the pro-drug hydrogels was observed under a reducing environment while cargo retention and integrity were maintained under physiological conditions. Gene silencing efficiency and cytocompatibility were optimized by screening the amine content of the particles. When appropriate control siRNA cargos were loaded into hydrogels, gene knockdown was only encountered for hydrogels containing releasable siRNAs, accompanied by minimal cell death.

Gene silencing via RNA interference (RNAi) (Fire, A.; Xu, S.; Montgomery, M. K.; Kostas, S. A.; Driver, S. E.; Mello, C. C. Nature 1998, 391, 806-811; Elbashir, S. M.; Lendeckel, W.; Tuschl, T. Genes Dev. 2001, 15, 188-200) has demonstrated great potential for treatment of diseases Leuschner, F. et al. Nat. Biotechnol. 2011, 1-9; Davis, M. E.; Zuckerman, J. E.; Choi, C. H. J.; Seligson, D.; Tolcher, A.; Alabi, C. A; Yen, Y.; Heidel, J. D.; Ribas, A. Nature 2010, 464, 1067-70) by halting the production of target proteins. The major challenge in realizing the potential of RNAi therapies resides in delivering small interfering RNA (siRNA) effectively to the cytoplasm of a target cell. With a highly negatively charged backbone and a molecular weight of ca. 13 kDa, siRNA is unable to effectively cross cell membranes without assistance. Additionally, siRNA is susceptible to degradation by ubiquitous RNases in serum. A suitable carrier is required to enhance stability and facilitate delivery to the cytoplasm of cells. Exemplar carriers include oligonucleotide conjugates (Oishi, M.; Nagasaki, Y.; Itaka, K.; Nishiyama, N.; Kataoka, K. J. Am. Chem. Soc. 2005, 127, 1624-5; Musacchio, T.; Vaze, 0.; D'Souza, G.; Torchilin, V. P. Bioconjugate Chem. 2010, 21, 1530-6; Kim, S. H.; Jeong, J. H.; Lee, S. H.; Kim, S. W.; Park, T. G. J. Controlled Release 2006, 116, 123-9; Lee, M.-Y.; Park, S.-J.; Park, K.; Kim, K. S.; Lee, H.; Hahn, S. K. ACS Nano 2011, 5, 6138-47; Cutler, J. I.; Zhang, K.; Zheng, D.; Auyeung, E.; Prigodich, A. E.; Mirkin, C. A. J. Am. Chem. Soc. 2011, 133, 9254-7; York, A. W.; Huang, F.; McCormick, C. L. Biomacromolecules 2010, 11, 505-14; Rozema, D. B.; Lewis, D. L.; Wakefield, D. H.; Wong, S. C.; Klein, J. J.; Roesch, P. L.; Bertin, S. L.; Reppen, T. W.; Chu, Q.; Blokhin, A. V.; Hagstrom, J. E.; Wolff, J. A. Proc. Natl. Acad. Sci. U.S.A. 2007, 104, 12982-7; Vázquez-Dorbatt, V.; Tolstyka, Z. P.; Chang, C.-W.; Maynard, H. D. Biomacromolecules 2009, 10, 2207-12; Nakagawa, O.; Ming, X.; Huang, L.; Juliano, R. L. J. Am. Chem. Soc. 2010, 132, 8848-9; Jeong, J. H.; Mok, H.; Oh, Y.-K.; Park, T. G. Bioconjugate Chem. 2009, 20, 5-14), polyplexes (Lee, M. Y., JACS, 2011); Allen, M. H.; Green, M. D.; Getaneh, H. K.; Miller, K. M.; Long, T. E. Biomacromolecules 2011, 12, 2243-50; Layman, J. M.; Ramirez, S. M.; Green, M. D.; Long, T. E. Biomacromolecules 2009, 10, 1244-52; Heidel, J. D.; Yu, Z.; Liu, J. Y.-C.; Rele, S. M.; Liang, Y.; Zeidan, R. K.; Kornbrust, D. J.; Davis, M. E. P Proc. Natl. Acad. Sci. U.S.A. 2007, 104, 5715-21; Convertine, A. J.; Benoit, D. S. W.; Duvall, C. L.; Hoffman, A. S.; Stayton, P. S. J. Controlled Release 2009, 133, 221-9), and lipoplexes (Akinc, A. et al. Nat. Biotechnol. 2008, 26, 561-9; Love, K. T. et al. Proc. Natl. Acad. Sci. U.S.A. 2010, 107, 1864-9; Semple, S. C. et al. Nat. Biotechnol. 2010, 28, 172-6; Li, S.-D.; Huang, L. Mol. Pharmaceutics. 2006, 3, 579-88). After systemic administration, the siRNA carrier encounters several biological hurdles en route to the target tissue and cell such as clearance by the reticuloendothelium system, protein fouling, and size requirements to reach particular tissues. Designing delivery vehicles with surface decorations including stealthing (e.g. polyethylene glycol, PEG (Klibanov, A. L.; Maruyama, K.; Torchilin, V. P.; Huang, L. FEBS Lett. 1990, 268, 235-7)) and targeting (e.g. peptide (Nakagawa, JACS, 2010)) ligands may promote prolonged circulation and passive delivery to tissues of interest followed by actively targeting cell surface receptors for internalization by desired cells.

Hydrogels and nanogels have been explored as delivery vector candidates for transfection of siRNA to target cells (Krebs, M. D.; Jeon, O.; Alsberg, E. J. Am. Chem. Soc. 2009, 131, 9204-6; Raemdonck, K.; Van Thienen, T. G.; Vandenbroucke, R. E.; Sanders, N. N.; Demeester, J.; De Smedt, S. C. Adv. Funct. Mater. 2008, 18, 993-1001). Hydrogel micro- or nano-particles may enable delivery of siRNA to a wide range of tissues in vivo in addition to unconventional locations like circulating cells. Particle Replication in Non-wetting Templates (PRINT®) technology allows for fabrication of hydrogels with control over size, shape, composition, surface chemistry, and modulus such that delivery properties may be tuned to particular applications (Rolland, J. P.; Maynor, B. W.; Euliss, L. E.; Exner, A. E.; Denison, G. M.; DeSimone, J. M. J. Am. Chem. Soc. 2005, 127, 10096-100; Petros, R. A.; Ropp, P. A.; DeSimone, J. M. J. Am. Chem. Soc. 2008, 130, 5008-9; Canelas, D. A.; Herlihy, K. P.; Desimone, J. M. Wiley Interdisciplinary Reviews: Nanomedicine and Nanobiotechnology 2009, 1, 391-404; Parrott, M. C.; Luft, J. C.; Byrne, J. D.; Fain, J. H.; Napier, M. E.; Desimone, J. M. J. Am. Chem. Soc. 2010, 132, 17928-32; Gratton, S. E. A; Ropp, P. A.; Pohlhaus, P. D.; Luft, J. C.; Madden, V. J.; Napier, M. E.; DeSimone, J. M. Proc. Natl. Acad. Sci. U.S.A. 2008, 105, 11613-8; Wang, J.; Tian, S.; Petros, R. A.; Napier, M. E.; Desimone, J. M. J. Am. Chem. Soc. 2010, 132, 11306-13; Enlow, E. M.; Luft, J. C.; Napier, M. E.; DeSimone, J. M. Nano Lett. 2011, 11, 808-13; Merkel, T. J.; Jones, S. W.; Herlihy, K. P.; Kersey, F. R.; Shields, A. R.; Napier, M. E.; Luft, J. C.; Wu, H.; Zamboni, W. C.; Wang, A. Z.; Bear, J. E.; DeSimone, J. M. Proc. Natl. Acad. Sci. U.S.A. 2011, 108, 586-91). Bottom-up approaches for encapsulating siRNA in nanogels through electrostatic attraction post-fabrication may result in dynamic association of cargo, uncontrollable cargo release, and modification of particle surface properties. Resulting concerns may be circumvented with PRINT technology, which allows for direct physical entrapment or covalent incorporation of siRNA during particle fabrication.

Presently, protein-based vaccines are commonly used for immunoprophylaxis of influenza virus infection. Although safety is an apparent advantage for protein-based vaccines, their usage is associated with a number of drawbacks, including low efficacy and short-term immunity because the injected protein is consumed in the immunogenic process. Nucleic acids offer a unique opportunity for vaccination and have emerged as excellent candidates for the treatment of cancers and infectious diseases (Tang D, DeVit M, Johnston S A (1992) Genetic immunization is a simple method for eliciting an immune response. Nature 356:152; Weide B, Garbe C, Rammensee H, Pascolob S (2008) Plasmid DNA- and messenger RNA-based anti-cancer vaccination Immunology Letters 115:33-42; Bringmann A et al. (2010) RNA Vaccines in Cancer Treatment. Journal of Biomedicine and Biotechnology 2010: 623-687; Kutzler M A and Weiner D B (2008) DNA vaccines: ready for prime time? Nature reviews genetics 9:777). Compared with traditional protein-based vaccination strategy, direct immunization with RNA or DNA has the advantages of the simplicity and purity with which they can be produced, as well as coding for a single protein of interest (i.e. an antigen) at high levels in a reproducible manner, potentially triggering immune response in both cellular and humoral branches (Cannon G and Weissman D (2002) RNA based vaccines. DNA and cell biology 21:953-961; Vajdy M et al. (2004) Mucosal adjuvants and delivery systems for protein-, DNA- and RNA-based vaccines Immunology and Cell Biology 82:617-627).

RNA replicon is an important form of nucleic acid-based vaccines and is derived from either positive- or negative-strand RNA viruses, from which the gene sequences encoding structural proteins are replaced by mRNA encoding antigens of interest as well as the RNA polymerase for RNA replicon replication and transcription (Anraku I et al. (2002) Kunjin virus replicon vaccine vectors induce protective CD8⁺ T-cell immunity. Journal of Virology 76:3791-3799; Tannis L L et al. (2005) Semliki forest virus and kunjin virus RNA replicons elicit comparable cellular immunity but distinct humoral immunity. Vaccine 23:4189-4194). RNA replicons can be regarded as “disabled” virus vectors that are capable of amplifying within the cytoplasm of host cells for a prolonged period but are unable to produce infectious progeny (Ying H et al. (1999) Cancer therapy using a self-replicating RNA vaccine. Nature Medicine 5:823-827; Diken M et al. (2011) Selective uptake of naked vaccine RNA by dendritic cells is driven by macropinocytosis and abrogated upon DC maturation. Gene Therapy 18:702-708). Compared with DNA vaccines, RNA replicon has several advantages: First, RNA replicon is capable of replicating in the cytoplasm of host cells, thus avoiding the requirement of nucleus entry which represents a daunting hurdle in DNA delivery (Lechardeur D et al. (1999) Metabolic instability of plasmid DNA in the cytosol: a potential barrier to gene transfer. Gene Therapy 6:482-497). By eliminating the dependence on cellular transcription machinery and transport of nucleic acids to and from the nucleus, RNA replicon is potentially a more efficient form of nucleic acid vaccine (Nishimura K et al. (2007) Persistent and stable gene expression by a cytoplasmic RNA replicon based on a noncytopathic variant sendai virus. The journal of biological chemistry 282:27383-27391). Secondly, RNA replicon has superior biosafety features, which is crucial for vaccine purposes. Compared with DNA, RNA replicon can avoid the potential integration into the genome of host cells and also prevent generation of anti-DNA antibodies, both of which may affect the host cell's gene expression in an uncontrollable manner and thus represent incalculable risks (Wang Z et al. (2004) Detection of integration of plasmid DNA into host genomic DNA following intramuscular injection and electroporation. Gene Therapy 11:711-721). RNA replicon combines the safety characteristics of inactivated vaccines with the superior immunogenicity of live, attenuated vaccines.

Studies have shown that RNA replicon-based vaccination is highly effective for generating cellular and protective immune responses, but has been delivered mainly as naked RNA transcribed in vitro or as RNA encapsidated into virus-like replicon particles (VLP) (Zimmer G (2010) RNA Replicons—A new approach for influenza virus immunoprophylaxis; Viruses 2:413-434; Kofler R M et al. (2004) Mimicking live flavivirus immunization with a noninfectious RNA vaccine. Proc Natl Acad Sci USA 101:1951-1956). The practical utility of VLP approach, however, is limited by manufacturing considerations, cost-effectiveness, and potential adverse health effects (Grgacic E V L, Anderson D A (2006) Virus-like particles: Passport to immune recognition, Methods 40:60-65; Ramsey J D, Vu H N, Pack D W (2010) A top-down approach for construction of hybrid polymer-virus gene delivery vectors. J Control Release, 144, 39-45).

The particle replication in non-wetting templates (PRINT) technique enables the generation of engineered micro- and nanoparticles having precisely controlled properties including size, shape, modulus, chemical composition and surface functionality for drug delivery applications (Wang J, Tian S, Petros R A, Napier M, DeSimone J M (2010) The complex role of multivalency in nanoparticles targeting the transferrin receptor for cancer therapies. J Am Chem Soc 132:11306-11313; Enlow E M, Luft J C, Napier M, DeSimone J M. (2011) Potent engineered PLGA nanoparticles by virtue of exceptionally high chemotherapeutic loadings. Nano Lett 11:808-813; Gratton S E A, et al. (2008) The effect of particle design on cellular internalization pathways. Proc Natl Acad Sci USA 105:11613-11618; Kelly J Y, DeSimone J M (2008) Shape-specific, monodisperse nano-molding of protein particles. J Am Chem Soc 130:5438-5439; Merkel T J et al. (2011) Using mechanobiological mimicry of red blood cells to extend circulation times of hydrogel microparticles. Proc Natl Acad Sci USA 108:586-591). PRINT is also amenable to continuous roll-to-roll fabrication techniques that enable the scale-up of the particle fabrication under good manufacturing practice (GMP) compliance.

### Protein-Based Particles

Delivering promising biological therapeutics to the desired location in the body in a safe and effective fashion is one of the key challenges in medicine. Protein-based therapies, which involve the delivery of therapeutic proteins or polypeptides, such as tumor necrosis factor, and monoclonal antibodies, is considered a safe and effective approach to treat many diseases ((a) Birch J. R.; Onakunle Y. Therapeutic Proteins, Methods and Protocols, 1-16 (Humana Press, 2005). (b) Johnson C. E.; Huang Y. Y.; Parrish A. B.; Smith M. I.; Vaughn A. E.; Zhang Q.; Wright K. M.; Van Dyke T.; Wechsler-Reya R. J.; Kornbluth S.; Deshmukh M. Proc. Natl. Acad. Sci. USA 2007, 104, 5220820-20825. (c) Chen B.; Erlanger B. F. Immunol. Lett. 2002, 84, 63-67). However, the impact of this strategy is limited by the low delivery efficiency to desired locations where proteins take action. In addition, drug carriers using proteins as matrices for the delivery of small molecule drugs and biological cargos, such as plasmid DNA and siRNA, are also being extensively studied ((a) Hawkins M. J.; Soon-Shiong P.; Desai N. Adv. Drug Deliv. Rev. 2008, 60, 876-885. (b) Rhaese S.; Briesen H.; Rubsamen-Waigmann H.; Kreuter J.; Langer K. J. Control. Release 2003, 92, 199-208. (c) Abbasi S.; Paul A.; Prakash S. Cell Biochem. Biophys. 2011, 61, 277-287). Each of these applications would benefit from having protein-based particles that dissolve slowly in a controlled and desirable manner. Herein, we report the synthesis of size- and shape-specific, biologically active protein micro- and nano-particles using a top-down particle fabrication technique called PRINT. Our approach involves the synthesis and design of a novel “traceless” cross-linking strategy that renders protein-based particles transiently insoluble in aqueous solutions.

Protein particles are often made through costly and complicated processes which include wet-milling, spray-freeze-drying, micro-emulsion, micro-encapsulation, or supercritical fluid methods ((a) Maa, Y. F.; Nguyen, P. A.; Sweeney, T.; Shire, S. J.; Hsu, C. C., Pharmaceut. Res. 1999, 16, 249-254. (b) Ma D.; Li M.; Patil A. J.; Mann S. Adv. Mater. 2004, 16, 1838-1841, (c) Carrasquillo K. G.; Carro J. C. A.; Alejandro A.; Toro D. D.; Griebenow K. J. Pharm. Pharmacol. 2001, 53, 115-120, (d) Dos Santos I. R.; Richard J.; Pech B.; Thies C.; Benoit J. P. Int. J. Pharm. 2002, 242, 69-78). Frequently, these procedures result in highly heterogeneous polydisperse spherical or granular particles and do not allow control over particle size or shape, resulting in significant heterogeneity of particle populations. Moreover, many of these processes are not compatible with optimal production of biological particles, as denaturation and aggregation of proteins tend to occur during processing. PRINT is a platform technology that is an off-shoot of soft lithography that enables the molding of micro- and nano-particles having precisely controlled size, shape, chemical composition and surface functionality ((a) Wang J.; Tian S.; Petros R. A.; Napier M. E.; DeSimone J. M. J. Am. Chem. Soc. 2010, 132, 11306-11313. (b) Gratton S. E. A.; Ropp P. A.; Pohlhaus P. D.; Luft J. C.; Madden V. J.; Napier M. E.; DeSimone J. M. Proc. Natl. Acad. Sci. USA 2008, 105, 11613-11618. (c) Merkel T. J.; Jones S. W.; Herlihy K. P.; Kersey F. R.; Shields A. R.; Napier M.; Luft J. C.; Wug H.; William C. Zambonic W. C.; Wang A. Z.; Bear J. E.; DeSimone J. M. Proc. Natl. Acad. Sci. USA 2010, 108, 586-591; Kelly J. Y.; DeSimone J. M. J. Am. Chem. Soc. 2008, 130, 5438-5543). PRINT has been transitioned to a continuous roll-to-roll fabrication technique that can enable the scale-up of particle production to practical levels for applications in the clinic.

Dry microspheres or nanospheres composed of proteins are usually instantaneously soluble when placed into aqueous solutions. A couple of strategies have been reported that maintains the stability of protein-based particles: i) thermal crosslinking, which causes the formation of intermolecular disulfide bridges between free thiol groups (Chatterjee J.; Haik Y.; Chen C. J. Colloid Polym. Sci., 2001, 279, 1073-1081); ii) the use of non-reversible chemical cross-linkers, such as glutaraldehyde, formaldehyde, 1-Ethyl-3-(3-dimethylaminopropyl)carbodiimide (EDC), etc, ((a) Arshady R. J. Control. Release, 1990, 14, 111-131. (b) Patil G. V. Drug Dev. Res., 2003, 58, 219-247); and iii) the use of reversible cross-linkers like Lomant's reagent, dithiobis[succinimidyl propionate] (DSP), which can be cleaved upon exposure to certain biologic conditions. Thermal cross-linking involves the thermal denaturation of a given protein at high temperature and cannot be applied to the delivery of functional therapeutic proteins. The use of non-reversible chemical cross-linkers introduces permanent cross-linkages between individual protein molecules which limits the release of free protein molecules. Such an approach has limited utility for the delivery of therapeutic proteins and biological cargos.

Reversible cross-linkers can be cleaved upon exposure to certain biologic conditions but leaves a chemical residue—a potential neo-epitope—on the protein after cleavage of the disulfide bond (Scheme α(a) ((a) Wu L. N. Y.; Fisher R. R. J. Biol. Chem., 1983, 258, 7847-7851. (b) Yu M.; Ng B. C.; Rome L. H.; Tolbert S. H.; Monbouquette H. G. Nano. Lett. 2008, 8, 3510-3515). If the released protein from the particles has molecular pendants attached, it may elicit undesirable immune responses towards foreign antigens, which may induce adverse health effects. For therapeutic proteins, very often, lysine residues are also involved in the active sites and modifying lysine residues with molecular pendants, if DSP is used, may abolish the protein activity.

Disclosed herein is a “traceless” reversible cross-linker that leaves no pendant chemical residues on the molecule it reacts with after cleavage of the linker. We have applied the use of such transient, trace-less chemical cross-linkers to achieve stabilization of protein particles fabricated using the PRINT technology. ((a) Jones L. R.; Goun E. A.; Shinde R.; Rothbard J. B.; Contag C. H.; Wender P. A. J. Am. Chem. Soc. 2006, 28, 6526-6527. (b) Dubikovskaya E. A.; Thorne S. H.; Pillow T. P.; Contag C. H.; Wender P. A. Proc. Natl. Acad. Sci. USA 2008, 105, 12128-12133).

The PRINT process utilizes the non-wetting properties of low surface energy molds to generate isolated particles via a unique soft lithography approach. Disclosed herein is a new approach for rendering these protein particle transiently insoluble (Kelly & DeSimone, JACS, 2008).

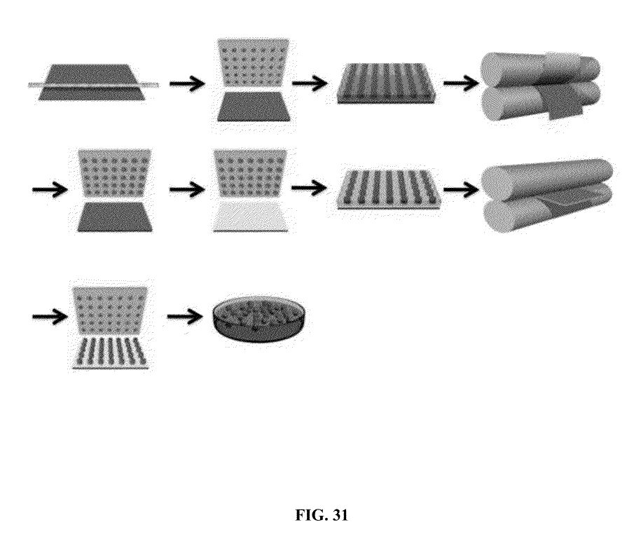

Serum albumin is the most abundant blood plasma protein it is essential for the transport of many physiological molecules and it also has the advantage of being readily available. Abraxane®, an albumin based paclitaxel containing nanomedicine, has achieved tremendous success as an approved treatment for metastatic breast cancer. (Hawkins M. J.; Soon-Shiong P.; Desai N. Adv. Drug Deliv. Rev., 2008, 60, 876-885). In particular, bovine serum albumin (BSA) was used in this study due to its easy accessibility and cost effectiveness for our proof-of-concept study. In this study, a melt-sodification strategy is employed (FIG. 31). Lactose and glycerol are mixed with the protein of choice, in this case BSA, to form the pre-particle material that readily “flows” when heated. Briefly, a film made from a mixture of BSA protein, lactose and glycerol on a high surface energy polyethylene terephthatlate (PET) sheet is heated in contact with a PRINT mold (mold # MMM-262-090A, MMM-369-070) while going through a pressured nip. In this step, the protein-lactose-glycerol mixture melts, flows into the cavities due to the capillary force and then solidifies as the mixture cools down to room temperature. The particles can then be transferred to a sacrificial adhesive layer, which can be dissolved in a good solvent for the adhesive layer and necessarily a poor solvent for the protein particle, in this case isopropanol, to release the PRINT particles from the surface. The processing temperature used to fabricate protein-based particles in this study can be as low as 60° C., which avoids the obvious degration of the delicate biological therapeutics that we are looking at.

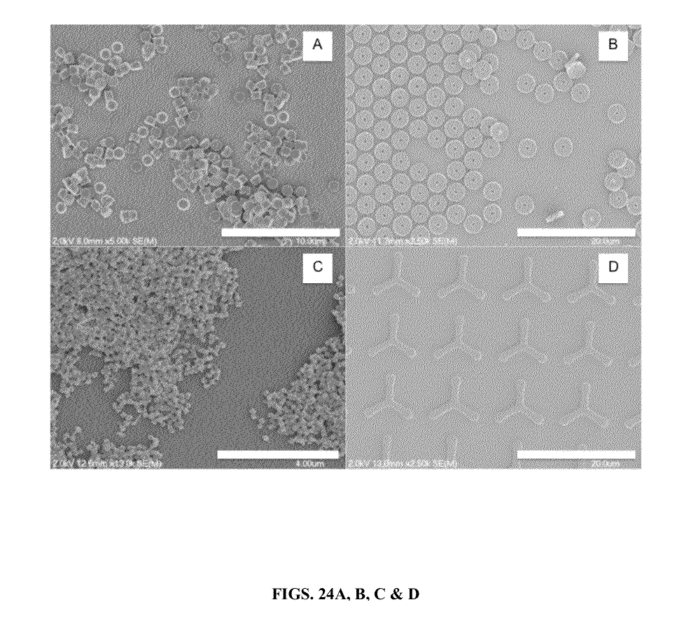

Taking advantage of the aforementioned PRINT process, a series of BSA particles were fabricated in the size range of 200 nm to several micrometers (FIG. 24). Cylindrical particles with both diameter and height as 1 μm were fabricated with a pre-particle composition containing 37.5 wt % of BSA, 37.5 wt % of α-D-lactose and 25.0 wt % of glycerol. After the harvest and purification steps using isopropanol, the dry particles were determined to contain 87.2±5.0 wt % of BSA based on a BCA protein assay and 7.4±1.6 wt % of lactose base on a lactose quantification assay, indicating the partial removal of the additives glycerol and lactose in the final harvested particle composition, respectively (Table A).

TABLE A
Particle composition

| | Charged | Final |
|---|---|---|
| | Composition ^{a} | Composition ^{b} |
| | (wt %) | (wt %) |
| BSA | 37.5 | 87.2 ± 5.0 |
| Lactose | 37.5 | 7.4 ± 1.6 |
| Glycerol | 25.0 | — |

^{a} The weight percentage of components charged into the pre-particle solution that was then drawn into a film on the PET sheet.
^{b} Final particle composition after harvest and purification step. The errors stand for standard deviation calculated from three experiments.

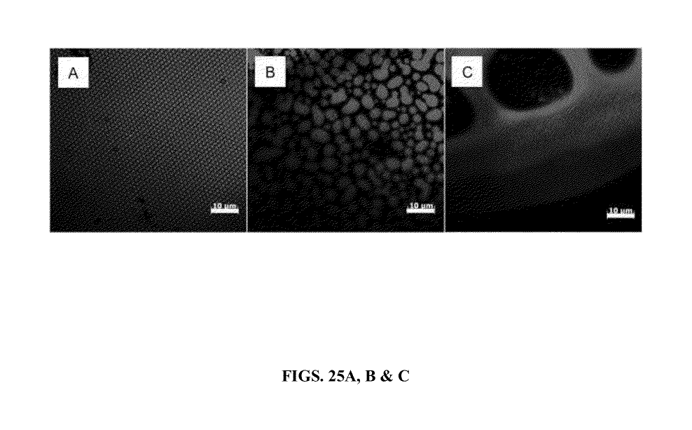

The protein particles, at this stage, are fully soluble when brought into contact with water. To monitor dissolution of BSA particles in water using fluorescent microscopy, 1 wt % of Alexa Fluor 555® dye labeled BSA was added to particles. Images were taken of the particles on the sacrificial adhesive layer before and after addition of water (FIG. 25). The particles dissolved instantaneously after exposure to water, which also illustrated that the molding process did not change the dissolution properties of albumin and indicated the necessity for a cross-linker to stabilize the albumin particles transiently.

In order to utilize protein-based particles for therapeutic applications, they are usually stabilized with cross-linkers, which can be cleaved under certain physiological stimuli. The cytoplasm of cells is known for its high concentration of reduced glutathione (GSH) compared to the extracellular environment (GSH concentration differs by 1000 folds intracellularly and extracellularly) (Saito G.; Swanson J. A.; Lee K. Adv. Drug Deliv. Rev. 2003, 55, 199-215).

Taking advantage of the reducing environment in the cytoplasm of cells, compounds, conjugates and cross-linkers have been prepared by introducing a disulfide-based cross-linker that should trigger the intracellular dissolution of our protein particles. Our initial studies using DSP to crosslink BSA particles indicated that di-N-hydroxysuccinimide (NHS) ester is highly reactive towards lysine residues on BSA and it is very difficult to control the crosslinking density of BSA particles, which is essential to achieve desired dissolution profiles. In addition, even though DSP is advertised as a reversible cross-linker, it is not a “truly” reversible cross-linker as it will leave molecular pendants after disulfide cleavage under reducing environment (Scheme α(a).

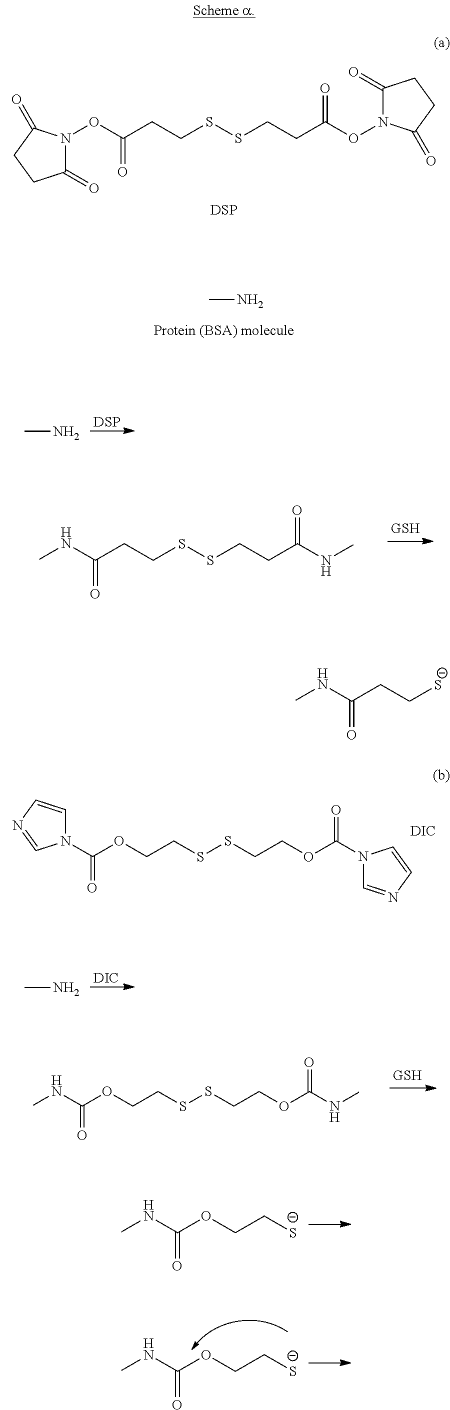

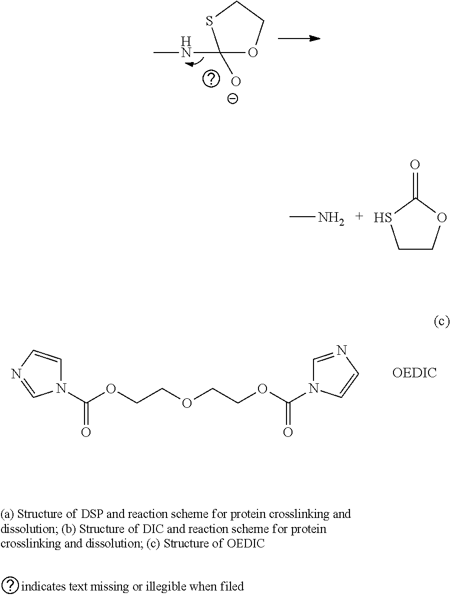

A truly reversible disulfide cross-linker with well-controlled reactivity was developed. Wender et al. developed a disulfide based pro-drug linker, which contains a carbonate group instead of conventionally used ester linkage, to release the drug in its original state ((a) Jones L. R.; Goun E. A.; Shinde R.; Rothbard J. B.; Contag C. H.; Wender P. A. J. Am. Chem. Soc. 2006, 28, 6526-6527. (b) Dubikovskaya E. A.; Thorne S. H.; Pillow T. P.; Contag C. H.; Wender P. A. Proc. Natl. Acad. Sci. USA 2008, 105, 12128-12133). This chemistry has also been applied to develop a fluorogenic probe for thiol detection and a pro-drug for intracellular delivery ((a) Namanja H. A.; Emmert D.; Davis D. A.; Campos C.; Miller D. S.; Hrycyna C. A.; Chmielewsk J. J. Am. Chem. Soc. 2011, (DOI: 10.1021/ja206867t). (b) Li C.; Wu T.; Hong C.; Zhang G.; Liu S. Angew. Chem. Int. Ed., 2012, 51, 455-459. (c) Pires M. M.; Chmielewski J. Org. Lett. 2008, 10, 837-840). Linkers having carbonate groups were developed, such as dithio-bis(ethyl 1H-imidazole-1-carboxylate) (DIC). Compared to DSP, DIC has several advantages. Imidazoles were introduced as the leaving groups in DIC to replace the highly reactive NHS as in DSP in order to better control the rate of the cross-linking reaction and the cross-linking density on the particle surface. Furthermore, DIC is a “traceless” reversible cross-linker, which does not leave any molecular pendants after disulfide cleavage (Scheme α(b)). Losing a stable five-membered ring structure may be a driving force for this reaction cascade.

According to some embodiments of the present invention, the drug concentration available at a target biologic system or location is increased through use of the linkage of the present invention. According to such embodiments, the present invention provides a system to covalently attach a drug to a particle for controlled or protected delivery. Covalently attaching the drug to the surface or interior of a particle, according to the present invention, eliminates diffusion of the drug out of or away from the particle. In some embodiments, by covalently attaching the drug to the particle ensures that the amount of drug charged (concentration before particle fabrication) and the amount of drug encapsulated (concentration after particle fabrication) are substantially similar. Typically, non-covalently encapsulated drugs can be washed away from the particle leading to a considerable difference between the amount of drug charged and the amount of encapsulated drug. Moreover, due to the covalent nature of the linkage, such linkage will provide particle-drug stability that is greater than the affinity binding (hydrogen bonding) found between avidin/biotin as a linker.

According to some embodiments of the present invention, utilizing the reversible disulfide chemistry reaction with the particle and/or its cargo for delivery to a target location can be tailored based on i) the degree of PEGylation, ii) the degree of lipidization, or iii) the degree of surface cross-linking. In further embodiments, the properties of a particle can be changed from hydrophobic to hydrophilic or from slowly degrading to rapidly degrading using identical reaction conditions. The rate of reduction of the disulfide bond can be tuned. The disulfide linker can be modified to provide steric hindrance in the vicinity of the disulfide bond. Accordingly, the reduction of the disulfide bond would be slowed due to the steric hindrance. In the linkers disclosed herein, large moieties at R^{a-h}, if present, more specifically at one or more positions R^{a-d}, can be added to provide the steric hindrance. Moieties such as propyl, isopropyl, butyl, t-butyl, and isobutyl can be used. Additionally, the A and B rings can be chosen accordingly to provide steric hindrance, as well as any substituents on the rings. As detailed herein, the disulfide linkers are completely reversible and all modifications to the particles and/or composition or component thereof will degrade under certain conditions resulting in the particles and/or composition or component thereof having the same structure as it was before conjugation.

In some embodiments, the present invention provides pro-drug linkages that are degradable under in vivo conditions, such as for example, in a reducing environment in the interior of a living cell. In some embodiment, the reversible nature of the linkages facilitates releasing the linked cargo for treating or diagnosing a target in vivo condition. Due to the reversible nature of these linkages, once the particle has reached a reducing environment the properties of the particle and/or the composition or component thereof has no remnant of the linker, i.e., the particle and/or component has the same structure and properties as before conjugation with the linker.

In some embodiments, the polymer is “PEG” or “poly(ethylene glycol)” as used herein, is meant to encompass any water-soluble poly(ethylene oxide). Typically, PEGs for use in the present invention will comprise the following structure: “—(CH₂CH₂O)ₙ—”. The variable (n) is 3 to 3000, and the terminal groups and architecture of the overall PEG may vary. PEGs having a variety of molecular weights, for example, from the low molecular weight of tetraethylene glycol to high molecular weight polymers of 100 kDa, structures or geometries as is known in the art. “Water-soluble”, in the context of a water soluble polymer is any segment or polymer that is soluble in water at room temperature. Typically, a water-soluble polymer or segment will transmit at least about 75%, more preferably at least about 95% of light, transmitted by the same solution after filtering. On a weight basis, a water-soluble polymer or segment thereof will preferably be at least about 35% (by weight) soluble in water, more preferably at least about 50% (by weight) soluble in water, still more preferably about 70% (by weight) soluble in water, and still more preferably about 85% (by weight) soluble in water. It is most preferred, however, that the water-soluble polymer or segment is about 95% (by weight) soluble in water or completely soluble in water.

An “end-capping” or “end-capped” group is an inert group present on a terminus of a polymer such as PEG. An end-capping group is one that does not readily undergo chemical transformation under typical synthetic reaction conditions. An end capping group is generally an alkoxy group, —OR, where R is an organic radical comprised of 1-20 carbons and is preferably lower alkyl (e.g., methyl, ethyl) or benzyl. “R” may be saturated or unsaturated, and includes aryl, heteroaryl, cyclo, heterocyclo, and substituted forms of any of the foregoing. When the polymer has an end-capping group comprising a detectable label, the amount or location of the polymer and/or the moiety (e.g., active agent) to which the polymer is coupled, can be determined by using a suitable detector. Such labels include, without limitation, fluorescers, chemiluminescers, moieties used in enzyme labeling, calorimetric (e.g., dyes), metal ions, radioactive moieties, and the like.

As used herein, the term “tracers” include, without limitation, fluorescers, chemiluminescers, moieties used in enzyme labeling, calorimetric (e.g., dyes), metal ions, radioactive moieties, and the like.

Lipids include natural or synthetic triglycerides or mixtures of same, monoglycerides and diglycerides, alone or mixtures of same or with e.g. triglycerides, self-emulsifying modified lipids, natural and synthetic waxes, fatty alcohols, including their esters and ethers and in the form of lipid peptides, or any mixtures of same.

Practice of the method of the present invention comprises administering to a subject a therapeutically effective amount of an agent containing a reversible disulfide linker or delivery vehicle comprising an agent containing a reversible disulfide linker as described herein.

Routes of administration for a therapeutically effective amount of an agent containing a reversible disulfide linker or delivery vehicle comprising an agent containing a reversible disulfide linker include but are not limited to intravenous or parenteral administration, oral administration, topical administration, transmucosal administration and transdermal administration. For intravenous or parenteral administration, i.e., injection or infusion, the composition may also contain suitable pharmaceutical diluents and carriers, such as water, saline, dextrose solutions, fructose solutions, ethanol, or oils of animal, vegetative, or synthetic origin. It may also contain preservatives, and buffers as are known in the art. When a therapeutically effective amount is administered by intravenous, cutaneous or subcutaneous injection, the solution can also contain components to adjust pH, isotonicity, stability, and the like, all of which is within the skill in the art. The pharmaceutical composition of the present invention may also contain stabilizers, preservatives, buffers, antioxidants, or other additive known to those of skill in the art. Typically, compositions for intravenous or parenteral administration comprise a suitable sterile solvent, which may be an isotonic aqueous buffer or pharmaceutically acceptable organic solvent. The compositions can also include a solubilizing agent as is known in the art if necessary. Compositions for intravenous or parenteral administration can optionally include a local anesthetic to lessen pain at the site of the injection. Generally, the ingredients are supplied either separately or mixed together in unit dosage form in a hermetically sealed container such as an ampoule or sachette. The pharmaceutical compositions for administration by injection or infusion can be dispensed, for example, with an infusion bottle containing, for example, sterile pharmaceutical grade water or saline. Where the pharmaceutical compositions are administered by injection, an ampoule of sterile water for injection, saline, or other solvent such as a pharmaceutically acceptable organic solvent can be provided so that the ingredients can be mixed prior to administration.

The duration of intravenous therapy using the pharmaceutical composition of the present invention will vary, depending on the condition being treated or ameliorated and the condition and potential idiosyncratic response of each individual mammal. The duration of each infusion is from about 1 minute to about 1 hour. The infusion can be repeated as necessary.

Systemic formulations include those designed for administration by injection, e.g., subcutaneous, intravenous, intramuscular, intrathecal or intraperitoneal injection. Useful injectable preparations include sterile suspensions, solutions or emulsions of the active compound(s) in aqueous or oily vehicles. The compositions also can contain solubilizing agents, formulating agents, such as suspending, stabilizing and/or dispersing agent. The formulations for injection can be presented in unit dosage form, e.g., in ampules or in multidose containers, and can contain added preservatives. For prophylactic administration, the compound can be administered to a patient at risk of developing one of the previously described conditions or diseases. Alternatively, prophylactic administration can be applied to avoid the onset of symptoms in a patient suffering from or formally diagnosed with the underlying condition.

The amount of compound administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, and the like. Determination of an effective dosage is well within the capabilities of those skilled in the art coupled with the general and specific examples disclosed herein.

Oral administration of the composition or vehicle can be accomplished using dosage forms including but not limited to capsules, caplets, solutions, suspensions and/or syrups. Such dosage forms are prepared using conventional methods known to those in the field of pharmaceutical formulation and described in the pertinent texts, e.g., in Remington: The Science and Practice of Pharmacy (2000), supra.

The dosage form may be a capsule, in which case the active agent-containing composition may be encapsulated in the form of a liquid. Suitable capsules may be either hard or soft, and are generally made of gelatin, starch, or a cellulosic material, with gelatin capsules preferred. Two-piece hard gelatin capsules are preferably sealed, such as with gelatin bands or the like. See, for e.g., Remington: The Science and Practice of Pharmacy (2000), supra, which describes materials and methods for preparing encapsulated pharmaceuticals.

Capsules may, if desired, be coated so as to provide for delayed release. Dosage forms with delayed release coatings may be manufactured using standard coating procedures and equipment. Such procedures are known to those skilled in the art and described in the pertinent texts (see, for e.g., Remington: The Science and Practice of Pharmacy (2000), supra). Generally, after preparation of the capsule, a delayed release coating composition is applied using a coating pan, an airless spray technique, fluidized bed coating equipment, or the like. Delayed release coating compositions comprise a polymeric material, e.g., cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, polymers and copolymers formed from acrylic acid, methacrylic acid, and/or esters thereof.

Sustained-release dosage forms provide for drug release over an extended time period, and may or may not be delayed release. Generally, as will be appreciated by those of ordinary skill in the art, sustained-release dosage forms are formulated by dispersing a drug within a matrix of a gradually bioerodible (hydrolyzable) material such as an insoluble plastic, a hydrophilic polymer, or a fatty compound. Insoluble plastic matrices may be comprised of, for example, polyvinyl chloride or polyethylene. Hydrophilic polymers useful for providing a sustained release coating or matrix cellulosic polymers include, without limitation: cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylcellulose phthalate, cellulose hexahydrophthalate, cellulose acetate hexahydrophthalate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate, with a terpolymer of ethyl acrylate, methyl methacrylate and trimethylammonioethyl methacrylate chloride (sold under the tradename Eudragit RS) preferred; vinyl polymers and copolymers such as polyvinyl pyrrolidone, polyvinyl acetate, polyvinylacetate phthalate, vinylacetate crotonic acid copolymer, and ethylene-vinyl acetate copolymers; zein; and shellac, ammoniated shellac, shellac-acetyl alcohol, and shellac n-butyl stearate. Fatty compounds for use as a sustained release matrix material include, but are not limited to, waxes generally (e.g., carnauba wax) and glyceryl tristearate.

Topical administration of an agent containing a reversible disulfide linker or delivery vehicle comprising an agent containing a reversible disulfide linker can be accomplished using any formulation suitable for application to the body surface, and may comprise, for example, an ointment, cream, gel, lotion, solution, paste or the like, and/or may be prepared so as to contain liposomes, micelles, and/or microspheres. Preferred topical formulations herein are ointments, creams, and gels.

Ointments, as is well known in the art of pharmaceutical formulation, are semisolid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy (2000), supra, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight (See, e.g., Remington: The Science and Practice of Pharmacy (2002), supra).

Creams, as also well known in the art, are viscous liquids or semisolid emulsions, either oil-in-water or water-in-oil. Cream bases are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

As will be appreciated by those working in the field of pharmaceutical formulation, gels-are semisolid, suspension-type systems. Single-phase gels contain organic macromolecules distributed substantially uniformly throughout the carrier liquid, which is typically aqueous, but also, preferably, contain an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the Carbopol® trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methylcellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

Various additives, known to those skilled in the art, may be included in the topical formulations. For example, solubilizers may be used to solubilize certain active agents. For those drugs having an unusually low rate of permeation through the skin or mucosal tissue, it may be desirable to include a permeation enhancer in the formulation; suitable enhancers are as described elsewhere herein.

Transmucosal administration of an agent containing a reversible disulfide linker or delivery vehicle comprising an agent containing a reversible disulfide linker can be accomplished using any type of formulation or dosage unit suitable for application to mucosal tissue. For example, an agent containing a reversible disulfide linker or delivery vehicle comprising an agent containing a reversible disulfide linker may be administered to the buccal mucosa in an adhesive patch, sublingually or lingually as a cream, ointment, or paste, nasally as droplets or a nasal spray, or by inhalation of an aerosol formulation or a non-aerosol liquid formulation.

Preferred buccal dosage forms will typically comprise a therapeutically effective amount of an agent containing a reversible disulfide linker or delivery vehicle comprising an agent containing a reversible disulfide linker and a bioerodible (hydrolyzable) polymeric carrier that may also serve to adhere the dosage form to the buccal mucosa. The buccal dosage unit is fabricated so as to erode over a predetermined time period, wherein drug delivery is provided essentially throughout. The time period is typically in the range of from about 1 hour to about 72 hours. Preferred buccal delivery preferably occurs over a time period of from about 2 hours to about 24 hours. Buccal drug delivery for short-term use should preferably occur over a time period of from about 2 hours to about 8 hours, more preferably over a time period of from about 3 hours to about 4 hours. As needed buccal drug delivery preferably will occur over a time period of from about 1 hour to about 12 hours, more preferably from about 2 hours to about 8 hours, most preferably from about 3 hours to about 6 hours. Sustained buccal drug delivery will preferably occur over a time period of from about 6 hours to about 72 hours, more preferably from about 12 hours to about 48 hours, most preferably from about 24 hours to about 48 hours. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver.

The “therapeutically effective amount” of an agent containing a reversible disulfide linker or delivery vehicle comprising an agent containing a reversible disulfide linker in the buccal dosage unit will of course depend on the potency and the intended dosage, which, in turn, is dependent on the particular individual undergoing treatment, the specific indication, and the like. The buccal dosage unit will generally contain from about 1.0 wt. % to about 60 wt. % active agent, preferably on the order of from about 1 wt. % to about 30 wt. % active agent. With regard to the bioerodible (hydrolyzable) polymeric carrier, it will be appreciated that virtually any such carrier can be used, so long as the desired drug release profile is not compromised, and the carrier is compatible with a reversible disulfide linker containing agent or delivery vehicle and any other components of the buccal dosage unit. Generally, the polymeric carrier comprises a hydrophilic (water-soluble and water-swellable) polymer that adheres to the wet surface of the buccal mucosa. Examples of polymeric carriers useful herein include acrylic acid polymers and co, e.g., those known as “carbomers” (Carbopol®, which may be obtained from B. F. Goodrich, is one such polymer). Other suitable polymers include, but are not limited to: hydrolyzed polyvinylalcohol; polyethylene oxides (e.g., Sentry Polyox® water soluble resins, available from Union Carbide); polyacrylates (e.g., Gantrez®, which may be obtained from GAF); vinyl polymers and copolymers; polyvinylpyrrolidone; dextran; guar gum; pectins; starches; and cellulosic polymers such as hydroxypropyl methylcellulose, (e.g., Methocel®, which may be obtained from the Dow Chemical Company), hydroxypropyl cellulose (e.g., Klucel®, which may also be obtained from Dow), hydroxypropyl cellulose ethers (see, e.g., U.S. Pat. No. 4,704,285 to Alderman), hydroxyethyl cellulose, carboxymethyl cellulose, sodium carboxymethyl cellulose, methyl cellulose, ethyl cellulose, cellulose acetate phthalate, cellulose acetate butyrate, and the like.

Other components may also be incorporated into the buccal dosage forms described herein. The additional components include, but are not limited to, disintegrants, diluents, binders, lubricants, flavoring, colorants, preservatives, and the like. Examples of disintegrants that may be used include, but are not limited to, cross-linked polyvinylpyrrolidones, such as crospovidone (e.g., Polyplasdone® XL, which may be obtained from GAF), cross-linked carboxylic methylcelluloses, such as croscarmelose (e.g., Ac-di-sol®, which may be obtained from FMC), alginic acid, and sodium carboxymethyl starches (e.g., Explotab®, which may be obtained from Edward Medell Co., Inc.), methylcellulose, agar bentonite and alginic acid. Suitable diluents are those which are generally useful in pharmaceutical formulations prepared using compression techniques, e.g., dicalcium phosphate dihydrate (e.g., Di-Tab®, which may be obtained from Stauffer), sugars that have been processed by cocrystallization with dextrin (e.g., co-crystallized sucrose and dextrin such as Di-Pak®, which may be obtained from Amstar), calcium phosphate, cellulose, kaolin, mannitol, sodium chloride, dry starch, powdered sugar and the like. Binders, if used, are those that enhance adhesion. Examples of such binders include, but are not limited to, starch, gelatin and sugars such as sucrose, dextrose, molasses, and lactose. Particularly preferred lubricants are stearates and stearic acid, and an optimal lubricant is magnesium stearate.

Sublingual and lingual dosage forms include creams, ointments and pastes. The cream, ointment or paste for sublingual or lingual delivery comprises a therapeutically effective amount of the selected active agent and one or more conventional nontoxic carriers suitable for sublingual or lingual drug administration. The sublingual and lingual dosage forms of the present invention can be manufactured using conventional processes. The sublingual and lingual dosage units are fabricated to disintegrate rapidly. The time period for complete disintegration of the dosage unit is typically in the range of from about 10 seconds to about 30 minutes, and optimally is less than 5 minutes.

Other components may also be incorporated into the sublingual and lingual dosage forms described herein. The additional components include, but are not limited to binders, disintegrants, wetting agents, lubricants, and the like. Examples of binders that may be used include water, ethanol, polyvinylpyrrolidone; starch solution gelatin solution, and the like. Suitable disintegrants include dry starch, calcium carbonate, polyoxyethylene sorbitan fatty acid esters, sodium lauryl sulfate, stearic monoglyceride, lactose, and the like. Wetting agents, if used, include glycerin, starches, and the like. Particularly preferred lubricants are stearates and polyethylene glycol. Additional components that may be incorporated into sublingual and lingual dosage forms are known, or will be apparent, to those skilled in this art (See, e.g., Remington: The Science and Practice of Pharmacy (2000), supra).

Other preferred compositions for sublingual administration include, for example, a bioadhesive to retain an agent containing a reversible disulfide linker or delivery vehicle comprising an agent containing a reversible disulfide linker sublingually; a spray, paint, or swab applied to the tongue; or the like. Increased residence time increases the likelihood that the administered invention can be absorbed by the mucosal tissue.

Transdermal administration of an agent containing a reversible disulfide linker or delivery vehicle comprising an agent containing a reversible disulfide linker through the skin or mucosal tissue can be accomplished using conventional transdermal drug delivery systems, wherein the agent is contained within a laminated structure (typically referred to as a transdermal “patch”) that serves as a drug delivery device to be affixed to the skin.

Transdermal drug delivery may involve passive diffusion or it may be facilitated using electrotransport, e.g., iontophoresis. In a typical transdermal “patch,” the drug composition is contained in a layer, or “reservoir,” underlying an upper backing layer. The laminated structure may contain a single reservoir, or it may contain multiple reservoirs. In one type of patch, referred to as a “monolithic” system, the reservoir is comprised of a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are separate and distinct layers, with the adhesive underlying the reservoir which, in this case, may be either a polymeric matrix as described above, or it may be a liquid or hydrogel reservoir, or may take some other form.

The backing layer in these laminates, which serves as the upper surface of the device, functions as the primary structural element of the laminated structure and provides the device with much of its flexibility. The material selected for the backing material should be selected so that it is substantially impermeable to the active agent and any other materials that are present, the backing is preferably made of a sheet or film of a flexible elastomeric material. Examples of polymers that are suitable for the backing layer include polyethylene, polypropylene, polyesters, and the like.

During storage and prior to use, the laminated structure includes a release liner. Immediately prior to use, this layer is removed from the device to expose the basal surface thereof, either the drug reservoir or a separate contact adhesive layer, so that the system may be affixed to the skin. The release liner should be made from a drug/vehicle impermeable material.

Transdermal drug delivery systems may in addition contain a skin permeation enhancer. That is, because the inherent permeability of the skin to some drugs may be too low to allow therapeutic levels of the drug to pass through a reasonably sized area of unbroken skin, it is necessary to coadminister a skin permeation enhancer with such drugs. Suitable enhancers are well known in the art and include, for example, those enhancers listed below in transmucosal compositions.

Formulations can comprise one or more anesthetics. Patient discomfort or phlebitis and the like can be managed using anesthetic at the site of injection. If used, the anesthetic can be administered separately or as a component of the composition. One or more anesthetics, if present in the composition, is selected from the group consisting of lignocaine, bupivacaine, dibucaine, procaine, chloroprocaine, prilocalne, mepivacaine, etidocaine, tetracaine, lidocaine and xylocalne, and salts, derivatives or mixtures thereof.

The present subject matter is further described herein by the following non-limiting examples which further illustrate the invention, and are not intended, nor should they be interpreted to, limit the scope of the invention.

### EXAMPLES

### 1. Reversible Lipidization of Particles

Reversible disulfide lipid conjugates are used to “lipidize” a polymer or the surface of nanoparticles or liposomes. Chemical modification by lipidization can improve oral bioavailability, minimize enzymatic degradation and cross blood brain barrier. Schemes 1 and 2 depict a general synthetic route to prepare lipid-modified, i.e., lipidized, polymers, nanoparticles and liposomes.

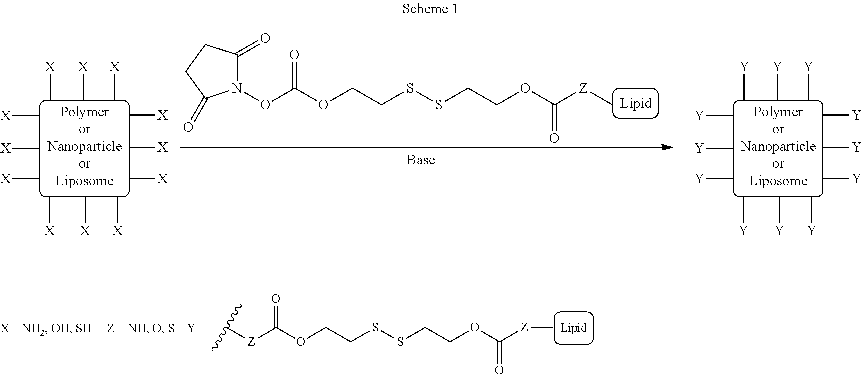

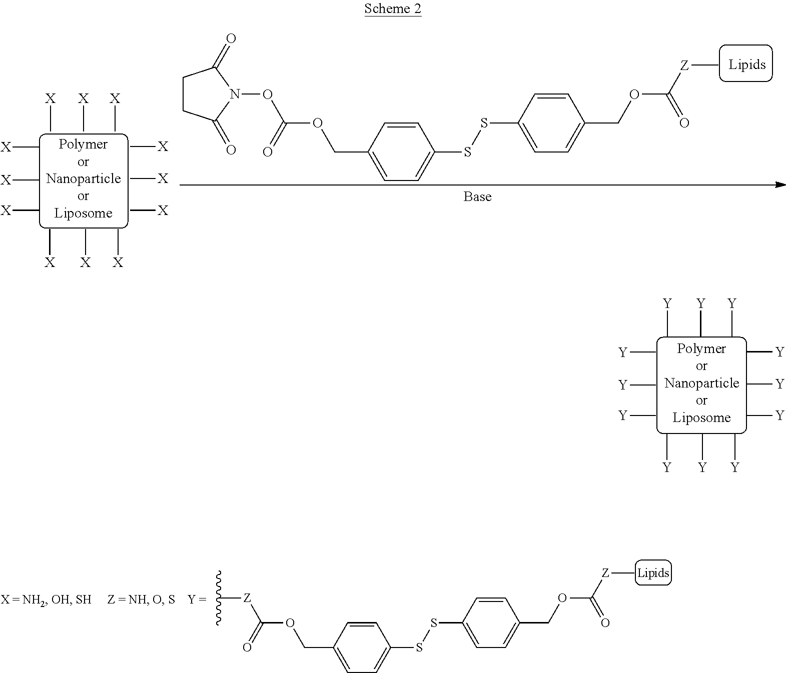

### 2. Reversible PEGylation of Nanoparticles

Reversible disulfide poly(ethylene glycol) conjugates are used to “PEGylate” a polymer or the surface of particles or liposomes. Chemical modification by PEGylation can improve water solubility, circulation in vivo, and the stealth properties of polymers, particles or liposomes. Schemes 3 and 4 depict a general synthetic route to prepare PEG-modified, i.e., PEGylated, polymers, nanoparticles and liposomes.

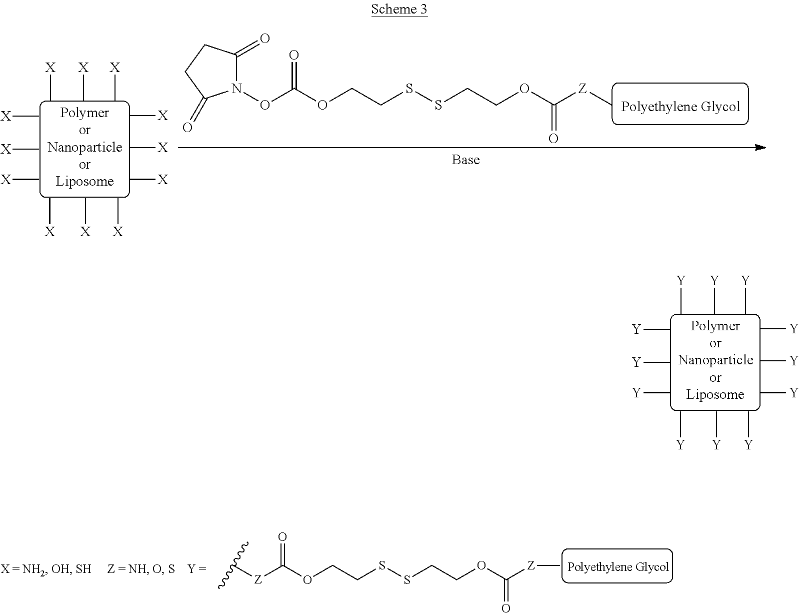

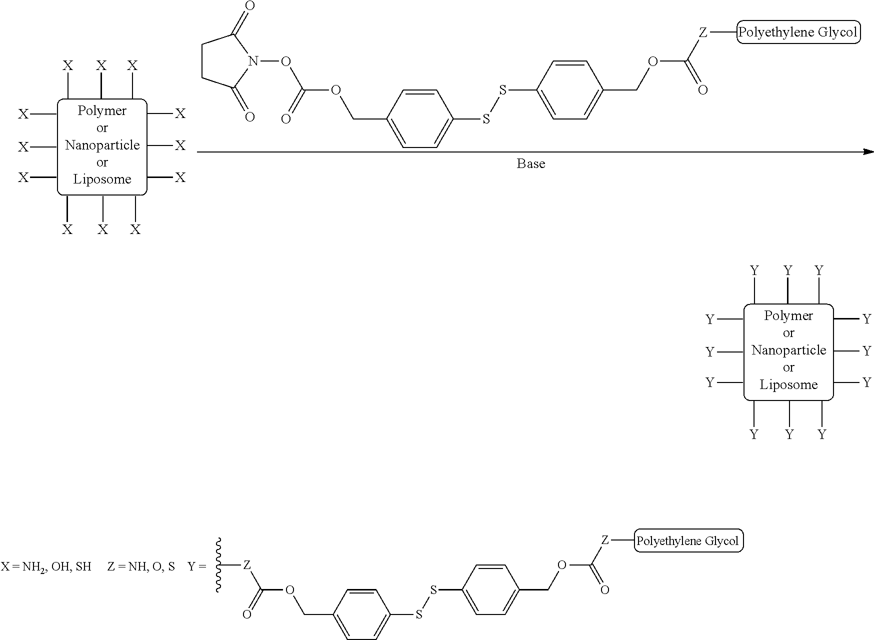

### 3. Modification of Polymers, Nanoparticles and Liposomes with Reversible Disulfide Pro-Drugs

Reversible disulfide modified agents and drugs (chemotherapeutics or biomolecules) are used to conjugate with polymers or coat the surface of nanoparticles or liposomes with a large payload of chemotherapeutics or biomolecules. The agent is attached by a reversible disulfide linkage to prepare a pro-drug, which can be degraded under intracellular reducing environments. This chemical modification can improve drug solubility, circulation, and ensure a large concentration reaches the desired tissue. Schemes 5 and 6 depict a general synthetic route to prepare pro-drug containing polymers, nanoparticles and liposomes.

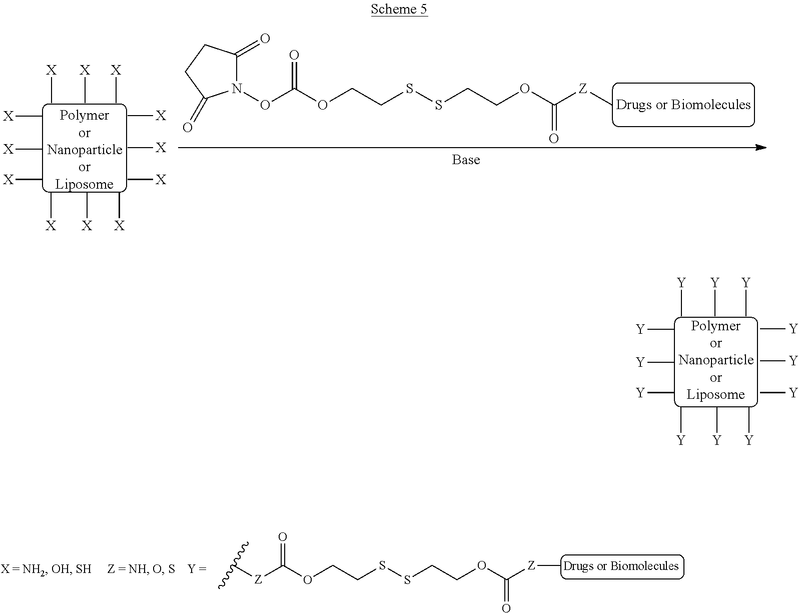

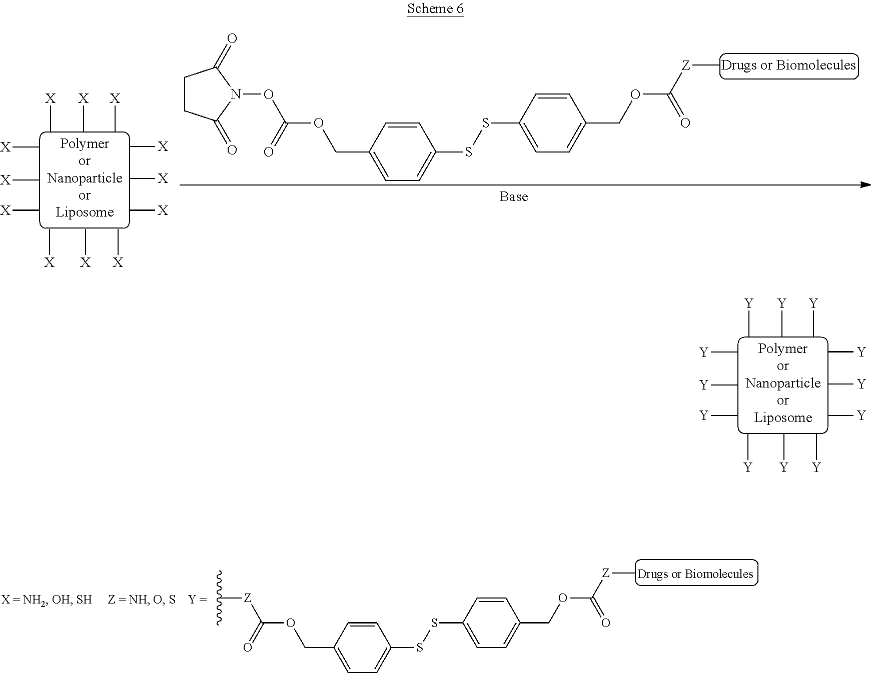

### 4. Shell Crosslinking of “Pure Protein” PRINT Particles with a Reversible Disulfide Crosslinker

Due to the versatile nature of the PRINT technology, nanoparticles can be fabricated with unprecedented high weight percentage of proteins (up to wt. 50%). To control the dissolution rates of protein particles in aqueous solutions, the surface of the nanoparticles can be crosslinked with a reversible disulfide linker, which can be degraded under intracellular reducing environment. Upon degradation of the reversible disulfide linker, the protein molecules can be fully restored to their original state, which can avoid eliciting immune response.

a. Nanoparticle Fabrication

The human serum albumin PRINT particles were derived from a mixture composed of 42 wt % of human serum albumin, 42 wt % of D-lactose and 16 wt % of glycerol. A 5 wt % solution of this mixture in water was prepared and then cast a film onto a poly(ethylene terephthalate) (PET) sheet. Water was removed with a heat gun. The transparent film was laminated onto a piece of fluorocur patterned mold (4×12 inch, cylindrical, d=200 nm, h=200 nm), forming a sandwich structure with the film in the middle. The mold was delaminated by passing the mold and the PET through a heated laminator with a temperature of 132° C. on the top roller and a pressure of 80 psi between the rollers. The filled mold was relaminated onto a sheet of luvitec covered PET. The laminated mold and PET were passed through the heated laminator again. The mold and the PET were separated gently and all the PRINT particles were transferred from the mold to the luvitec film. The particles were harvested from the PET by dissolving the luvitec with isopropanol. The harvested particles were washed with isopropanol for three times by centrifugation to remove luvitec. The particles were finally dispersed in isopropanol and the particle concentration was determined by Thermal Gravimetric Analysis (TGA).

Based on the TGA result, an appropriate amount of isopropanol was added to the particle dispersion to achieve a particle concentration of 0.5 mg/mL. To 1 mL of particle dispersion, 2 mg of the reversible disulfide crosslinker Dithio-bis(ethyl 1H-imidazole-1-carboxylate) (DIC) was added (compound I). The resulting dispersion was shaken on a vortexer for 24 hours at 37° C. The reaction was terminated by centrifuging particles at 14000 rpm for 5 minutes, followed by removal of the supernatant containing the crosslinker and addition of 1 mL of isopropanol. The particles were washed twice with isopropanol by centrifugation to remove the excess crosslinkers and then resuspended in water.

b. Dissolution Studies

For dissolution studies, bovine serum (BSA), Alexa Fluor® 555 conjugate was incorporated into the particles and the release of the dye-conjugated protein was used to characterize the dissolution rate of the particles. Typically, particles were fabricated from a mixture of 40 wt % of human serum albumin, 2 wt % of albumin from bovine serum (BSA), Alexa Fluor® 555 conjugate, 42 wt % of D-lactose and 16 wt % of glycerol. The particles were crosslinked and then resuspended in water following the procedures described above. The particle concentration in water was determined by TGA. An appropriate amount of water was added to the particle dispersion to achieve a particle concentration of 1 mg/mL. To each mini dialysis unit (purchased from Fisher Scientific, MWCO 7K), 50 μL of particle solution was added.

Typically, 3 units were dialyzed against 500 mL of Phosphate Buffer Saline solution (PBS) containing 5 mM glutathione with a magnetic bar stirring gently at the bottom of the beaker. Another 3 units were dialyzed against 500 mL of PBS buffer without glutathione as controls. The dialysis process was carried out in a 37° C. incubator. At different time points (8 h, 24 h, 48 h), one unit was withdrawn from each bath. The particle solution was recovered from the units and each unit was washed with 1004 of PBS. The wash was combined with recovered particle solution and appropriate amount of PBS was added to achieve a total mass of 200 mg. The solution was centrifuged at 14000 rpm for 10 minutes.

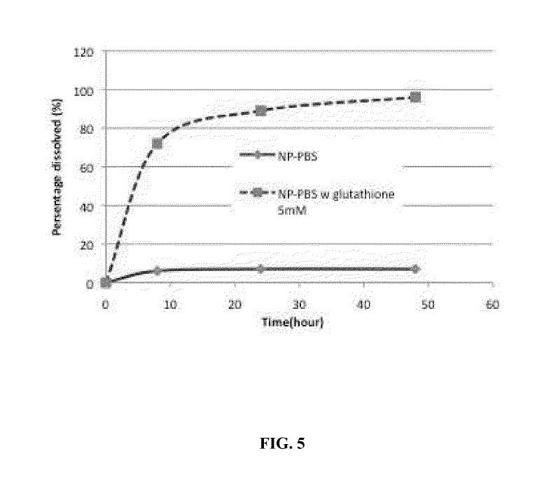

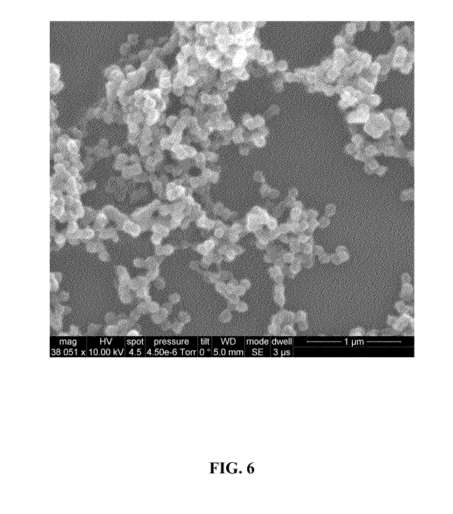

The supernatant was measured for fluorescence (excitation 545 nm, emission 575 nm) by a SpectraMax M5 plate reader (Molecular Devices). The fluorescence from PBS was used as background and the fluorescence from uncrosslinked particles (0.25 mg/mL in PBS) was used as 100% control. The dissolution profile is shown in FIG. 5. Crosslinked particles that were exposed to PBS only remained intact over the 48 hr time period, while the particles exposed to PBS with the reducing agent glutathione were fully degraded at 48 hours. FIG. 6 shows an ESEM image of particles that were crosslinked with reversible disulfide crosslinker Dithio-bis(ethyl 1H-imidazole-1-carboxylate) (DIC) for 24 hours at 37° C. Particles remain intact following incubation in water. Albumin particles with no crosslinker would fall apart immediately in water.

c. Nanoparticle Cell Uptake

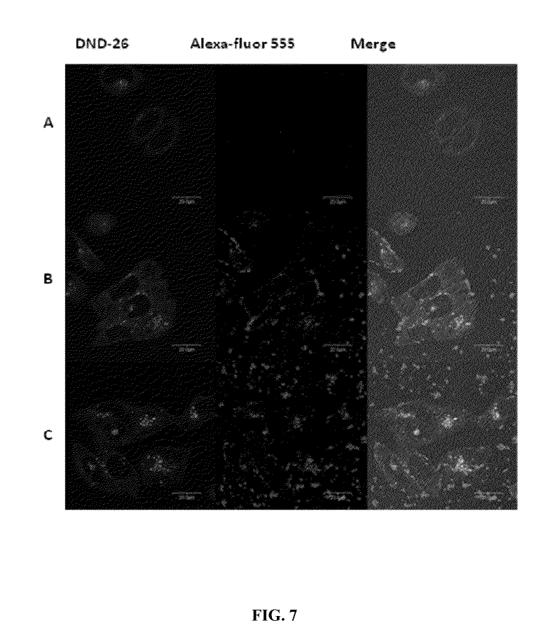

To facilitate internalization by cells, polyethyleneimine (PEI, branched Mw 22K) was incorporated into the particles and confocal laser scanning microscopy was used to monitor uptake of particles into the cells. Typically, the particles containing 2 wt % of PEI were fabricated from a mixture of 38 wt % of human serum albumin, 2 wt % of PEI, 2 wt % of albumin from bovine serum (BSA), Alexa Fluor® 555 conjugate, 42 wt % of D-lactose and 16 wt % of glycerol. The particles containing 4 wt % of PEI were fabricated from a mixture of 36 wt % of human serum albumin, 4 wt % of PEI, 2 wt % of albumin from bovine serum (BSA), Alexa Fluor® 555 conjugate, 42 wt % of D-lactose and 16 wt % of glycerol. The particles were crosslinked and then resuspended in water following the procedures described above. The particle concentration in water was determined by TGA. For cell uptake test, HeLa cells (5000 cells/well in a glass bottom 96-well plate, MatTek Corp.) were treated dosed with PRINT nanoparticles at 50 μg/mL in OPTI-MEM for 4 h at 37° C. (5% CO₂), and during the last 2 h simultaneously cells were also simultaneously treated with 1200 nM Lysotracker green (DND-26) for 2 h at 37° C. (5% CO₂). Cells were then washed 3 times with DPBS to remove particles and Lysotracker dye, and resuspended replaced in DMEM with 10% FBS in a glass bottom 96-well plate (MatTek Corp.) for imaging with an Olympus Fluorview FV500 confocal laser scanning microscope (Olympus) in the UNC Microscopy Services Laboratory. Cell uptake profile is shown in FIG. 7. Particles fabricated without PEI showed no uptake and no adhesion to cells. At 4 h dosing, particles with PEI were taken up by cells and mostly co-localized with DND-26 (yellow spots), which stains acidic intracellular vesicles. Some red particles were seen separate from DND-26, suggesting that they are in cytosol. Red particles on the cell edges most probably are on the surface of cells.

### 5. Reversible Asymmetric Disulfide Pro-Drugs and Biologics

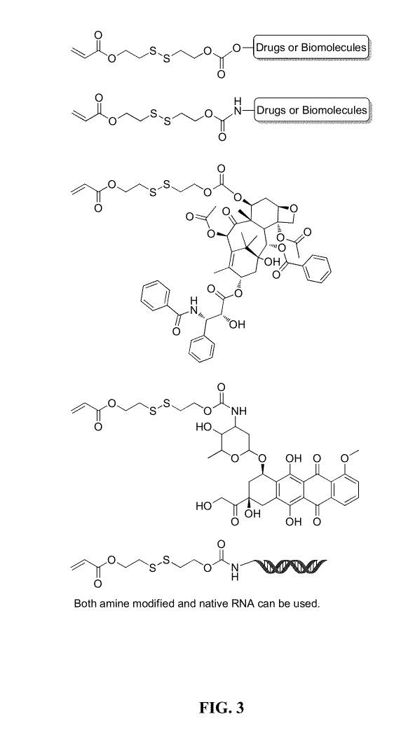

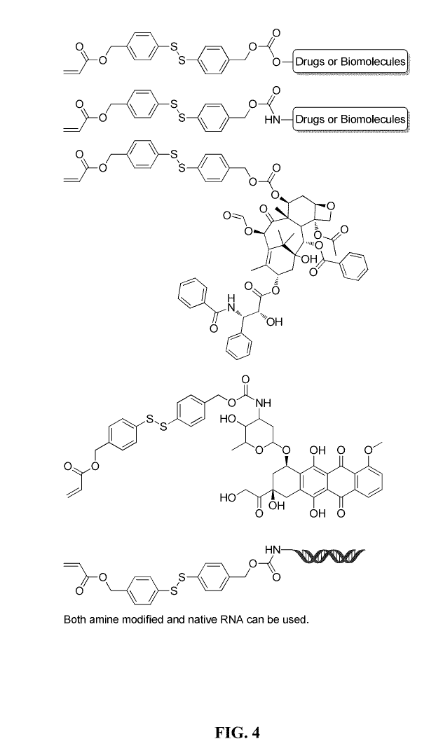

A polymerizable reversible disulfide is used to incorporate therapeutics to form a polymer or within the interior of nanoparticles or liposomes. The therapeutics can be i) a drug/chemotherapeutic, ii) a protein, iii) a peptide, or iv) a nucleic acid (DNA, RNA, siRNA, shRNA, miRNA and RNA replicon etc.). See FIGS. 3 and 4. The therapeutic is attached by a reversible disulfide linkage, which can be degraded under intracellular reducing environment. The therapeutic can be released in its native form without any molecular pendants. This chemical modification can improve the solubility and circulation of the therapeutic, and will ensure a large concentration of the therapeutic reaches the desired tissue. Additionally, having the drug inside the polymer/particle provides protection, which minimizes untimely degradation.

a. Reversible Disulfide siRNA Conjugate

The synthesis of the siRNA prodrug conjugate is shown in Scheme 7 using anti-luciferase and irrelevant siRNA. Mass Spectrometry (nanoelectrospray ionization) characterization confirmed the structure of the conjugate.

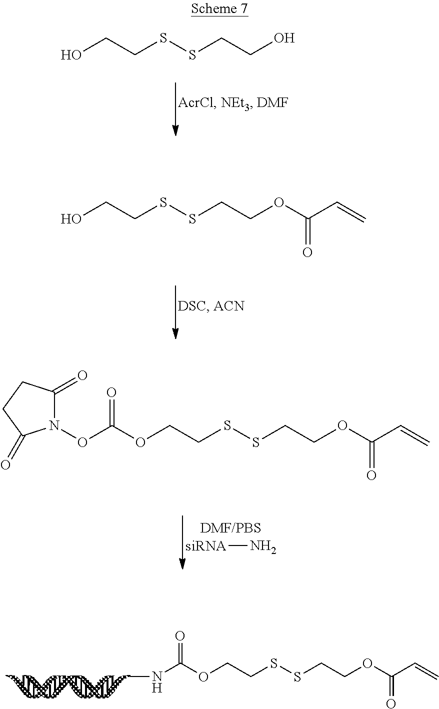

b. Reversible Disulfide siRNA Conjugate Activity

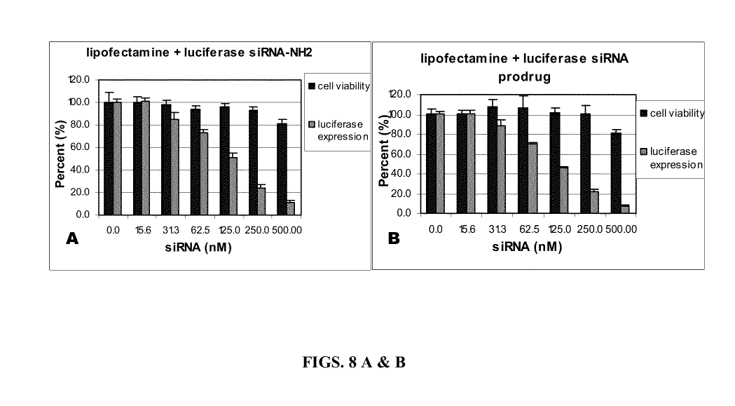

The activity of anti-luciferase siRNA was tested in vitro using a HeLa cell line stably transfected with firefly luciferase reporter gene to ensure that the activity was unchanged following the modification of the siRNA to form the reversible disulfide conjugate. The amine-terminated (native) and prodrug siRNA were dosed on HeLa cells and transfected with Lipofectamine transfection reagent. The siRNA conjugates were allowed to remain on the cells for 4 hours followed by further incubation for 2 days at 37° C. in cell media. Knockdown of luciferase expression was evaluated by measuring bioluminescence. Native and prodrug siRNA elicited comparable knockdown (FIG. 8) while gene silencing was absent when dosing control (inactive or irrelevant) siRNA. Maintenance of prodrug siRNA activity indicates compatibility of the oligonucleotide with reaction and purification conditions involved in prodrug synthesis.

### 6. Preparation of Reversible Disulfide Pro-Drug siRNA Hydrogels PRINT Particles

Particles were fabricated using the compositions given in Table 1. Particle characterization is described in Table 2. To fabricate the particles, the first step is to make a pre-particle solution. The components and weight percentages of each of the components is given in Table 1. Three different particle compositions were fabricated and the wt % of each component listed is considered to be the charged amount of that component. For the three particle compositions, the wt % of the AEM and the HP4A was altered. AEM is a cationic moiety that can be used to change the zeta potential of the particle. The positive charge of the particle increases as the AEM concentration is increased. Addition of AEM and positive charge aids in cell internalization of the particle. The component were added in a stepwise fashion to DEPC-treated water to prepare the pre-particle solution. All remaining particle fabrication steps were conducted in a humidity room (70% relative humidity). Using a #5 wire wound rod (R.D.S.), 150 μL of pre-particle solution was cast at 6 ft/min on PET, followed by brief evaporation of solvent with heat gun to yield a transparent film (delivery sheet). 200×200 nm cylindrical Fluorocur-patterned PRINT molds (Liquidia Technologies) were laminated against the delivery sheet with moderate pressure and then gently delaminated. The filled mold was laminated against corona-treated PET and subsequently cured in a UV chamber (λmax=365 nm, 90 mW/cm2) for 5 min After photocuring, the mold was removed to reveal an array of particles on PET. Particles were harvested off PET with water mechanically using a cell scraper (1 mL/48 in2). Supernatant was removed via centrifugation (5 min, 14 k rpm, 4° C.) and particles were washed twice with PBS at 0.5 mg/mL for 20 min.

TABLE 1
Particle Compositions

| Component | wt % | wt % | wt % |
|---|---|---|---|
| PEG₁₆-DA | 5 | 5 | 5 |
| HP₄A | 73 | 58 | 28 |
| AEM | 5 | 20 | 50 |
| Poly(vinyl alcohol) | 10 | 10 | 10 |
| Irgacure 2959 | 1 | 1 | 1 |
| Fluorescein o-acrylate | 1 | 1 | 1 |
| siRNA | 5 | 5 | 5 |

TABLE 2
Particle Characterization

| Particle, siRNA | ζ-potential/mV | D_{z}/nm |
|---|---|---|
| 5% AEM, L | +34.0 ± 1.9 | 350.2 ± 5.4 |
| 20% AEM, L | +49.1 ± 0.7 | 281.6 ± 1.9 |
| 20% AEM, C | +49.0 ± 1.5 | 261.4 ± 8.7 |
| 50% AEM, L | +49.2 ± 2.4 | 253.7 ± 3.4 |

L = Anti-Luciferase siRNA
C = Irrelevant siRNA or control siRNA

### Abbreviations:

AcrCl—acryloyl chloride

NEt₃—triethylamine

DSC—disuccinimidyl carbonate

ACN—acetonitrile

siRNA-NH2—5′-amine-modified siRNA

DMF—N,N-dimethylformamide

PBS—phosphate buffered saline

HP4A—hydroxy-PEG4-acrylate

PEG16DA—PEG16-diacrylate

PVA—poly(vinyl alcohol), 2 kDa

AEM—2-aminoethyl methacrylate hydrochloride

TBE—Tris/Borate/EDTA

### 7. Activity of Reversible Disulfide Pro-Drug siRNA Hydrogels PRINT Particles

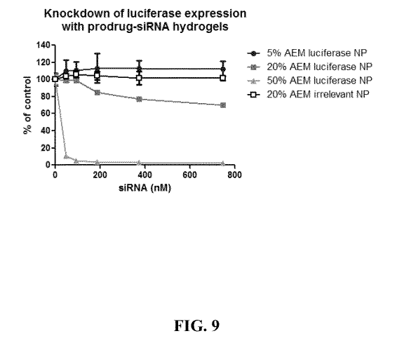

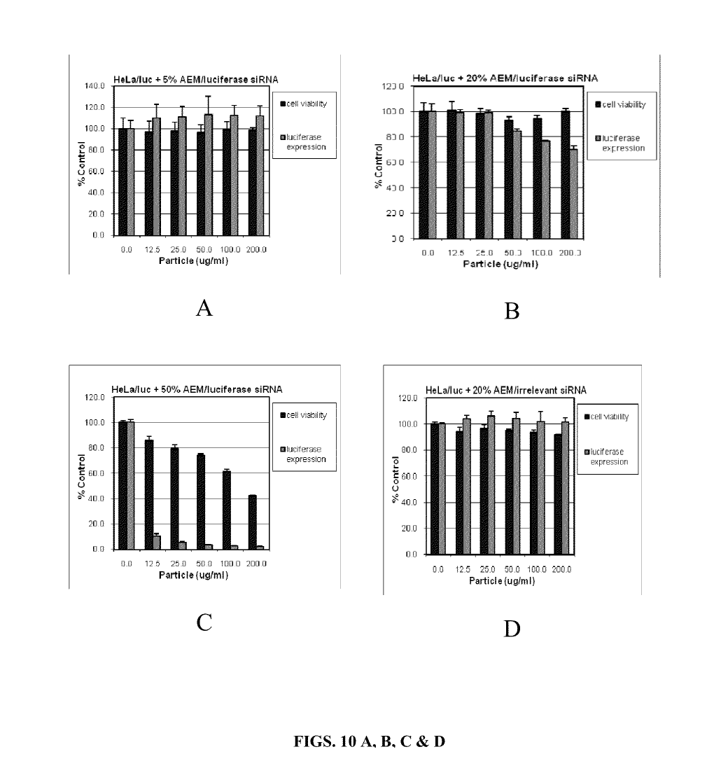

Particles were then dosed onto HeLa cells that have been stably transfected with luciferase. The particles were allowed to remain on the cells for 4 hours and then removed. The cells were incubated further for 48 hours and then analyzed for knockdown of luciferase activity. The siRNA concentration was calculated assuming 5 wt % siRNA final encapsulation (charged amount). 30% knockdown of luciferase expression was observed with hydrogels containing 20 wt % AEM, and >90% knockdown was observed with 50 wt % AEM at the highest particle concentrations (FIG. 9). No knockdown was observed for the particle containing anti-luciferase siRNA and 5 wt % AEM. In addition no knockdown was observed for irrelevant control. There was dose-dependent toxicity associated with 50 wt % AEM (FIG. 10); a further screening of AEM content between 20 and 50 wt % should reveal the best composition for cationic prodrug-siRNA hydrogels.

### 8. Synthesis of a Reversible Disulfide Linker

Synthesis of compound 3. To a 100 mL flask, N,N′-disuccinimidyl carbonate (DSC, 5.12 g, 20.0 mmol) was dissolved in 50 mL of CHCl₃. 2,2′-Dithiodiethanol (0.308 g, 2.0 mmol) was dissolved in 20 mL of CHCl₃ and added dropwise to the DSC solution. The reaction was kept at room temperature for 12 hr and diluted with 100 mL of CHCl₃. The organic phase was washed with NaCl saturated ice water for three times and dried with sodium sulfate. The final product, compound 3 was purified using chromatography.

### 9. A. Transfection of Cancer Cells with siRNA Electrostatically Entrapped in Hydrogels

A highly cationic, moderately crosslinked hydrogel composition (Table B) was synthesized to allow for physical entrapment and electrostatic association of siRNA within cylindrical (diameter [d]=200 nm; height [h]=200 nm) PRINT particles.

TABLE B
Composition of pre-particle solution for preparation
of siRNA-containing cationic hydrogels nanoparticles.

| Component | Function | wt % |
|---|---|---|
| PEG_{1K} dimethacrylate | crosslinker | 23 |
| mPEG_{5K} acrylate | hydrophile | 20 |
| 2-aminoethyl methacrylate HCl | cationic handle | 50 |
| 1-hydroxycyclohexylphenyl ketone | photo initiator | 1 |
| Fluorescein o-acrylate | fluorescent tag | 1 |
| siRNA | cargo | 5 |

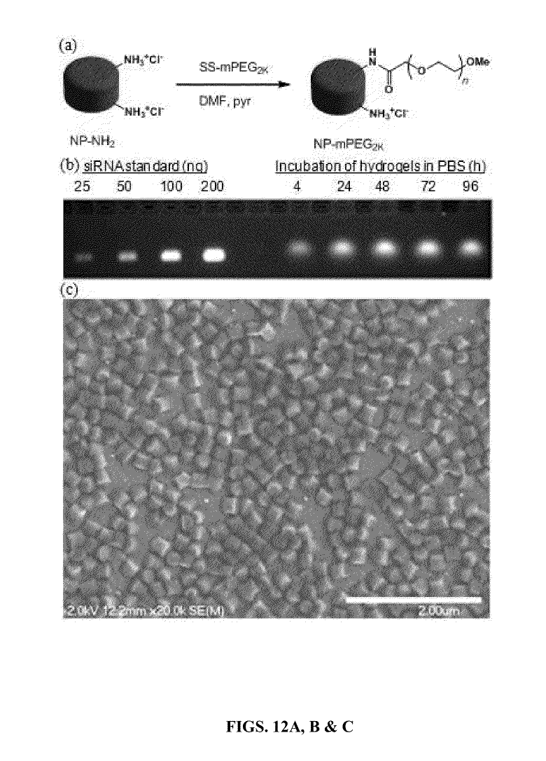

To promote cytocompatibility and dispersibility of highly cationic hydrogel nanoparticles in aqueous media, amine handles on hydrogels were reacted with succinimidyl succinate monomethoxy PEG_{2K} (FIG. 12a). After PEGylating the hydrogels, a concomitant decrease in the ζ-potential was observed (Table C), resulting in a low surface charge that would be minimally toxic to cell membranes.

TABLE C
Zetasizer analysis of siRNA-charged hydrogels
before and after PEGylation.

| Particle (siRNA) | ζ-potential (mV) | D_{z} (nm) |
|---|---|---|
| NP-NH₂ (luciferase) | +16.6 ± 0.38 | 438.8 ± 8.9 |
| NP-NH₂ (control) | +17.6 ± 0.55 | 445.0 ± 10.4 |
| NP-mPEG_{2K} (luciferase) | +10.2 ± 0.42 | 390.9 ± 5.0 |
| NP-mPEG_{2K} (control) | +9.72 ± 0.29 | 391.9 ± 8.5 |

SEM analysis of the hydrogel particles demonstrates their cylindrical shape and dimensions (FIG. 12b). Steady release of siRNA from the hydrogel particles in PBS at 37° C. was observed over time, reaching maximum concentration around 48 h (FIG. 12c). By gel electrophoresis, encapsulation efficiency of siRNA in hydrogels was determined to be ca. 28% (1.4 wt % siRNA loading in particles).

### B. In Vitro Studies of siRNA Electrostatically Entrapped in Hydrogels

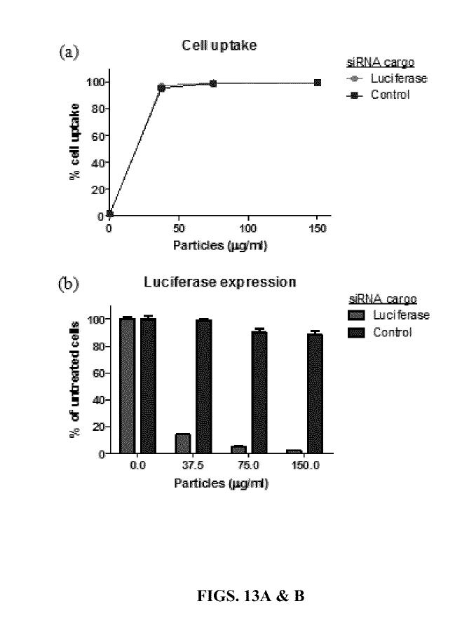

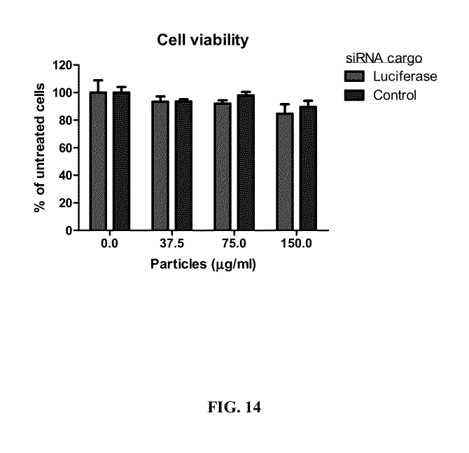

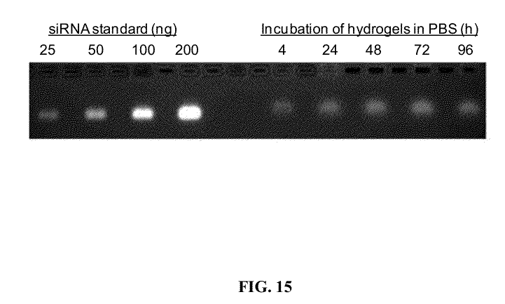

To evaluate the transfection potential of particles loaded with siRNA, a stably-transfected, luciferase-expressing human cervical cancer (HeLa) cell line was utilized for in vitro studies. Particles were dosed on HeLa cells for 4 h followed by 72 h incubation. Due to the positive charge of the particles, the PRINT hydrogel particles were readily internalized into the HeLa cells (FIG. 13a) as determined by flow cytometry. Dose-dependent knockdown of luciferase expression (FIG. 13b) was observed for HeLa cells incubated with the anti-luciferase siRNA-charged particles with a half-maximal effective concentration (EC₅₀) of ca. 6 nM siRNA. Conversely, PRINT hydrogel particles charged with a control siRNA sequence did not elicit gene knockdown, implying that transfection was sequence-specific (FIG. 13b). Additionally, both particles were found to be cytocompatible with HeLa cells (FIG. 14). Even though the desired cell activity was achieved with these particles, subsequent efforts to modulate in vivo behavior by conjugation of targeting ligands to the particle surface can result in premature release of siRNA. For example, 50% loss of encapsulated cargo was observed during a sequence of reactions to add targeting moieties to the particle surface (FIG. 15).

### C. Pro-siRNA Incorporation into Hydrogels

In order to combat the premature release issues with the PRINT hydrogel particles described above, an alternative pro-drug strategy was employed which involved covalently conjugating the siRNA directly to the PRINT hydrogel particles. Disulfide-siRNA conjugates to polymers and lipids have been previously reported^{6,7,10-12} as reductively-labile systems. In this work, siRNA was derivatized with a photopolymerizable acrylate bearing a degradable disulfide linkage for reversible covalent incorporation to the PRINT hydrogel nanoparticles. In the designed ‘pro-siRNA hydrogels’, it was envisioned that the siRNA cargo would be retained in the particle until entry of the particle into the cytoplasm of a cell, where the disulfide linkage would be cleaved in the reducing environment, allowing for release and delivery of the siRNA.

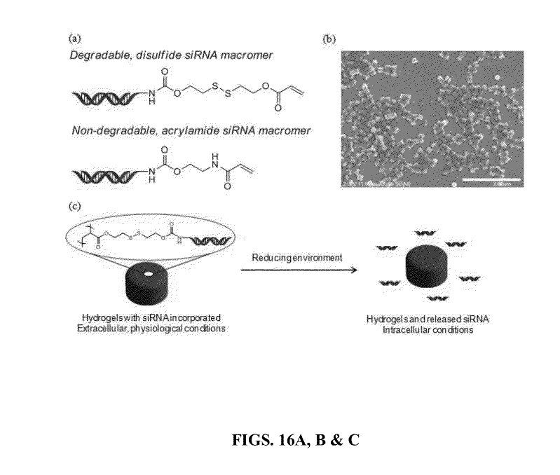

Disulfide-containing siRNA macromers were synthesized (FIG. 16a) for this pro-drug siRNA delivery vehicle along with non-disulfide acrylamide-based hydrogel PRINT particle as a control system. The disulfide-based siRNA pro-drug was included as part of the pre-particle solution (Table D) to fabricate cylindrical (d=200 nm; h=200 nm) loosely-crosslinked cationic PRINT hydrogel particles using a film-split technique followed by extensively washing of the particles to remove the sol fraction.

TABLE D
Compositions of pre-particle solution
for fabrication of pro-siRNA hydrogels.

| Component | Function | wt % |
|---|---|---|
| PEG₇₀₀ diacrylate | crosslinker | 5 |
| Tetraethyleneglycol monoacrylate | hydrophile | 73-28 |
| 2-aminoethyl methacrylate HCl | cationic handle | 5-50 |
| Poly(vinyl alcohol) 2 kDa | porogen and PET-wetter | 10 |
| Irgacure 2959 | photoinitiator | 1 |
| DyLight 488 maleimide | fluorescent dye | 1 |
| siRNA | cargo | 5 |

A water-based pre-particle solution with higher content of hygroscopic, liquid monomers was applied to the pro-siRNA hydrogel system to achieve conversion of siRNA macromer. SEM micrograph of the pro-drug PRINT hydrogel particles confirmed the dimensions and shape of cylindrical siRNA-containing particles (FIG. 16b). The intended behavior of the siRNA conjugate in cylindrical hydrogel nanoparticles is illustrated in FIG. 16c where siRNA remains bound to the matrix until entering a reducing environment such as that found in the cytoplasm of a cell.

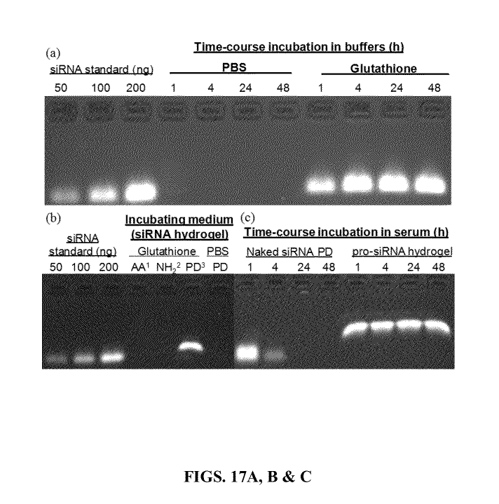

The time-dependent release of the siRNA from the pro-drug PRINT hydrogel particles was evaluated under physiological and reducing conditions (FIG. 17a). The siRNA was retained in the hydrogel particles over 48 h at 37° C. in PBS while the siRNA was quickly released from hydrogels when incubated in a reducing environment (5 mM glutathione), reaching maximum concentration around 4 h (FIG. 17a). Moreover, the siRNA conjugate did not even leak out of the hydrogel particles when they were exposed to high salt concentration buffer (10×PBS) at 37° C. for 4 h (FIG. 17b). Incubation of the non-disulfide, non-degradable acrylamide control siRNA hydrogel particles did not result in release of the siRNA under reducing conditions as expected. Stability of the siRNA covalently conjugated to the PRINT hydrogel particles was tested by incubation of the particles in serum (10% FBS) as a function of time. Over 48 h, the siRNA in the pro-drug PRINT hydrogel particles could be protected from degradation by RNases when incubated in serum while siRNA in the form of the simple macromonomer in the absence of the particle to protect it was rapidly degraded in serum under the same conditions (FIG. 17c).

### D. Pro-siRNA Hydrogels for Gene Silencing

The PRINT particles were designed to have a positive zeta potential to facilitate cell internalization and endosomal escape by including an amine monomer (AEM, 2-aminoethylmethacrylate hydrochloride). It is known that excessive amine content in hydrogels may disrupt and destroy the plasma membrane, eliciting cell death. Conversely, an insufficient amine content may not enable efficient cell uptake and endosomal escape for transfection. To optimize cytocompatibility and gene silencing efficiency of pro-siRNA hydrogels, the AEM content was varied from 5 to 50 wt % (Table E).

TABLE E
Zetasizer analysis of pro-siRNA hydrogels
with variable amine content.

| Amine content (wt %) | ζ-potential (mV) | D_{z} (nm) |
|---|---|---|
| 5% AEM | +18.2 ± 0.5 | 350.2 ± 5.4 |
| 20% AEM | +22.6 ± 0.1 | 281.6 ± 1.9 |
| 25% AEM | +27.1 ± 0.3 | 307.4 ± 6.6 |
| 30% AEM | +27.9 ± 1.5 | 324.3 ± 5.6 |
| 40% AEM | +30.6 ± 1.0 | 281.3 ± 6.0 |
| 50% AEM | +34.1 ± 0.4 | 253.7 ± 3.4 |

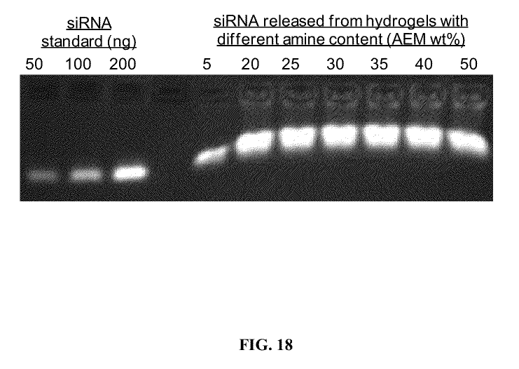

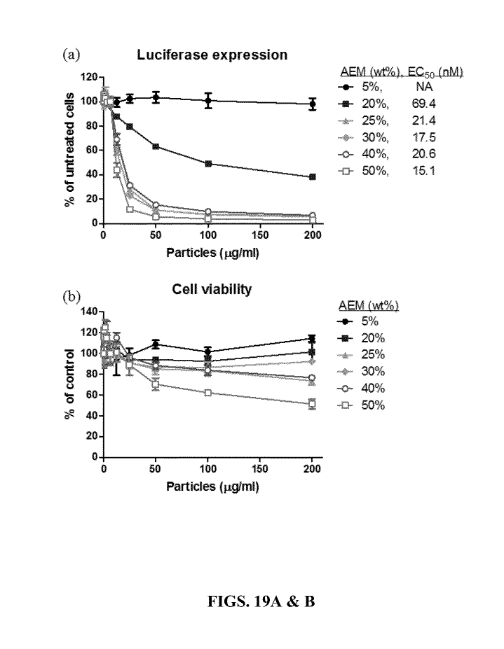

ζ-potentials of cationic hydrogels increased with amine content and the diameters of the resultant particles ranged from 250 to 350 nm (Table E). Encapsulation of the siRNA in the hydrogel PRINT particles reached a roughly constant value once the amine content was greater than or equal to 20 wt % (FIG. 18). The encapsulation efficiency was determined to be ca. 35% for AEM contents ≧20 wt % while when only using 5% AEM, the encapsulation was lower (ca. 15%). When the pro-drug, disulfide-containing siRNA hydrogel PRINT particles were dosed onto Luciferase-transfected HeLa cells (HeLa/luc) for 5 h followed by 48 h incubation at 37° C., dose-dependent knockdown of the luciferase expression was observed (FIG. 19a) for hydrogels with amine contents greater than 5 wt % AEM. Cytocompatibility was maintained at the lower amine contents and dosing concentrations (FIG. 19b). It appeared that the 30% AEM-containing PRINT hydrogel particles provided the ideal combination of gene silencing efficiency (EC₅₀˜20 nM siRNA) and cytocompatibility (even at high dosing concentrations).

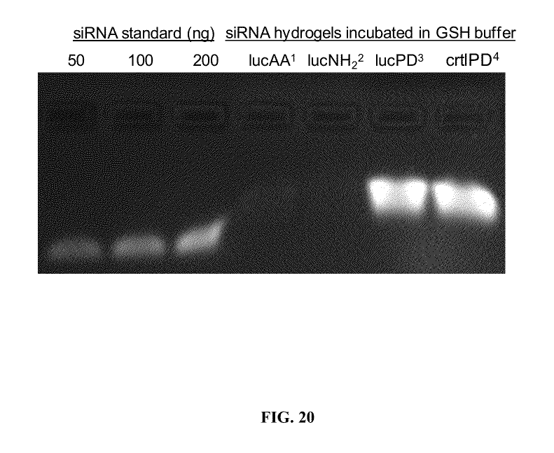

To further investigate the in vitro gene knockdown efficacy of the PRINT hydrogel particles, the 30% AEM-based hydrogel composition was utilized with four different cargos: (1) native luc siRNA, (2) degradable disulfide luc siRNA, (3) non-degradable, acrylamide luc siRNA, and (4) degradable disulfide control siRNA. Zetasizer analysis of the hydrogel PRINT particles indicated that their size and charge were similar (Table F) and gel electrophoresis (FIG. 20) allowed for confirmation of the encapsulation of the various cargos.

TABLE F
Zetasizer analysis of cationic hydrogels
charged with different siRNAs.

| siRNA cargo | ζ-potential (mV) | D_{z} (nm) |
|---|---|---|
| Luc prodrug | +17.3 ± 0.2 | 340.0 ± 7.0 |
| Luc AA prodrug | +16.7 ± 0.2 | 292.6 ± 6.4 |
| Luc NH₂ | +20.8 ± 0.8 | 304.2 ± 6.7 |
| Crtl prodrug | +17.4 ± 1.0 | 325.6 ± 1.2 |

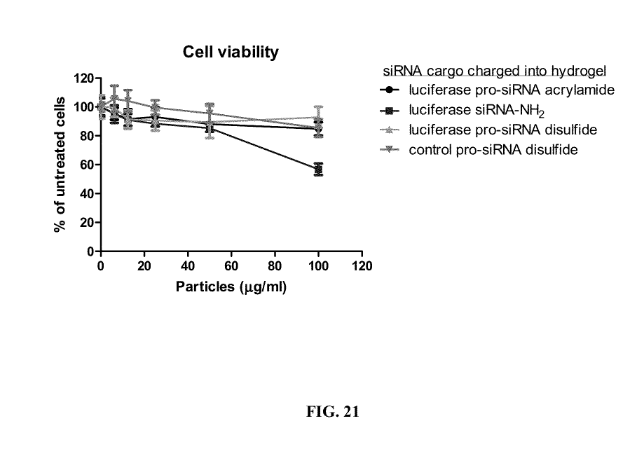

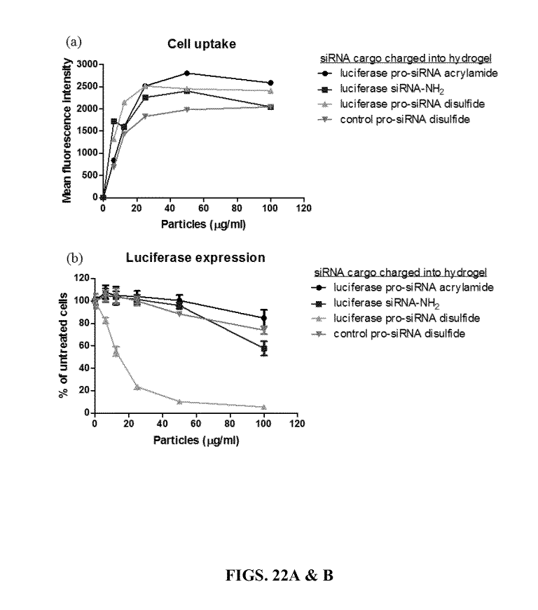

After dosing the particles on cells and incubating for 48 h, cell viability was maintained above 80% for all of the samples across all dosing concentrations, except for the particles containing the free siRNA (FIG. 21). Uptake of all of the hydrogel particles approached saturation at around 50 μg/mL particle concentration (FIG. 22a). Dose-dependent silencing of luciferase expression was elicited notably for the pro-drug, disulfide-based siRNA-containing hydrogel particles while the control particles did not elicit significant gene knockdown (FIG. 22b).

### Experimental

Materials. 2,2′-dithiodiethanol, acryloyl chloride, PEG₇₀₀ diacrylate, disuccinimidyl carbonate (DSC), 2-aminoethyl methacrylate hydrochloride (AEM), and Irgacure 2959 were purchased from Sigma Aldrich. Poly(vinyl alcohol) 75% hydrolyzed MW≈2 kDa was obtained from Acros Organics. Tetraethylene glycol monoacrylate (HP₄A) was synthesized in-house and kindly provided by Dr. Matthew C. Parrott, Dr. Ashish Pandya, and Mathew Finniss PRINT molds were graciously supplied by Liquidia Technologies. siRNAs were purchased as duplexes from Dharmacon, Inc. Sense sequence of amine-modified and native anti-luciferase siRNA: 5′-N6-GAUUAUGUCCGGUUAUGUAUU-3′; anti-sense: 5′-P-UACAUAACCGGACAUAAUCUU-3′. Sense sequence of amine-modified and native control siRNA: 5′-N6-AUGUAUUGGCCUGUAUUAGUU-3′; anti-sense: 5′-P-CUAAUACAGGCCAAUACAUU-3′. All other reagents were obtained from Fisher Scientific.

Synthesis of siRNA macromers. Degradable disulfide macromer precursor: 2,2′-dithiodiethanol (15 mL, 0.12 mol) was dissolved in anhydrous DMF (250 mL) in a 500-mL round-bottomed flask containing NEt₃ (20.5 mL, 1.2 eq) under a N₂ blanket to which acryloyl chloride (11.0 mL, 1.1 eq) was added dropwise and allowed to react for 8 h. Crude product was extracted into dichloromethane against 5% LiCl and purified via silica gel chromatography (EtOAc:hexanes) to provide monoacrylate-substituted 2,2′-dithiodiethanol (63% yield). 2-((2-hydroxyethyl)disulfanyl)ethyl acrylate (10 g, 48 mmol) was dissolved in anhydrous acetonitrile (100 mL) in a N₂-purged 250-mL round-bottomed flask, followed by addition of disuccinimidyl carbonate (14.8 g, 1.2 eq). The reaction proceeded for 8 h and product was purified by silica gel chromatography (EtOAc:hexanes 4:1) to afford 2-N-hydroxysuccinimide, 2′-acryloyl-dithiodiethanol as a clear, viscous liquid (82% yield). ¹H NMR (600 MHz, CDCl₃) δ=6.46 (dd, J=17.4 Hz, 1H), δ=6.2 (dd, J=10.7 Hz, 6.8 Hz, 1H), δ=5.90 (dd, J=10.7 Hz, 1H), δ=4.59 (t, J=6.5 Hz, 2H), δ=4.45 (t, J=6.5 Hz, 2H), δ=3.05-3.00 (m, J=6.8 Hz, 4H), δ=2.87 (s, 4H).

Non-degradable siRNA conjugate precursor: N-hydroxyethyl acrylamide (15 mL, 0.14 mol) was dissolved with DSC (51.9 g, 1.4 eq) in ACN:DMF 4:1 (250 mL) and reacted for 16 h. Afterward, ACN was removed via rotary evaporation and product was extracted into EtOAc against 5% LiCl. Product was concentrated and purified by silica gel column chromatography (EtOAc:hexanes 4:1) to provide 2-(succinimidyl carbonate)ethyl acrylamide (80% yield) as a fine white solid. ¹H NMR (600 MHz, CDCl₃) δ=6.50 (br, 1H, NH), δ=6.34 (dd, J=17.1 Hz, 1H), δ=6.19 (dd, J=10.3 Hz, 6.4 Hz, 1H), δ=5.71 (dd, J=10.3 Hz, 1H), δ=4.46 (t, J=5.0, 2H), δ=3.71 (m, J=5.5 Hz, 2H), δ=2.87 (s, 4H).

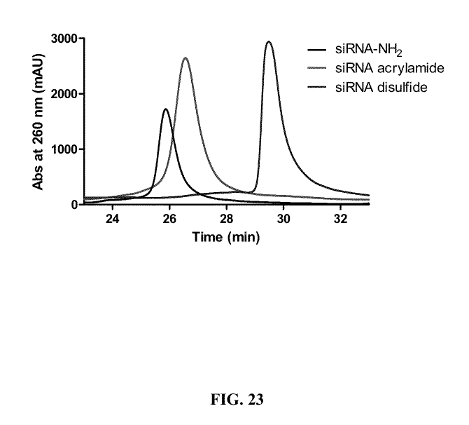

siRNA macromers: siRNA-NH₂ (2 mg, 148 nmol, luciferase) was dissolved in DEPC-treated PBS (200 μL) in a 1.5-mL RNAse-free Eppendorf tube. Separately, 2-N-hydroxysuccinimide, 2′-acryloyl-dithiodiethanol (5.2 mg, 100 eq) or 2-(succinimidyl carbonate)ethyl acrylamide (3.8 mg, 100 eq) was dissolved in RNAse-free DMF (150 μL) and added to the solution of siRNA. The reaction was allowed to proceed for 36 h where additional 100 eq of the acrylate or acrylamide were added to the reaction mixture every 12 h. 5 M NH₄OAc (50 μL) and EtOH (1.1 mL) were added to the reaction mixture, which was vortexed for 15 sec. The sample was incubated in a −80° C. freezer for 4 h followed by centrifugation (14 krpm, 4° C., 20 min) to pellet the siRNA. The supernatant was decanted and the pellet was washed twice with 70% EtOH (ice-cold) to provide siRNA prodrug (79% yield). HR-ESI-MS: m/z found for siRNA sense strand [M−H]⁻=6832.366; m/z calc. for disulfide macromer [M−H]⁻=7067.676; found [M−H]⁻=7067.855; m/z calc. for siRNA acrylamide macromer [M−H]⁻=6974.506; found [M−H]⁻=6974.871. Characterization of siRNA prodrug precursors was carried out on a 600 MHz Bruker NMR Spectrometer equipped with a Cryoprobe and siRNA macromonomers were analyzed by an IonSpec Fourier Transform Mass Spectrometer FTMS (20503 Crescent Bay Drive, Lake Forest, Calif. 92630) with a nano electrospray ionization source in combination with a NanoMate (Advion 19 Brown Road, Ithaca, N.Y. 14850) chip based electrospray sample introduction system and nozzle operated in the negative ion mode as well as reversed phase high-performance liquid chromatography (FIG. 23).

Fabrication of hydrogels via PRINT process. Pre-particle solutions were prepared with listed compositions at 2.5 wt % in RNase-free DMF (for physically entrapped siRNA) or DEPC-treated water containing 0.01% sodium dodecyl sulfate (for prodrug siRNA, where all remaining steps were conducted in a humidity room maintained at 70% relative humidity). Using a #5 wire wound rod (R.D.S.), 150 μL of pre-particle solution was cast at 6 ft/min on a sheet of poly(ethylene terephthalate) (PET), followed by brief evaporation of solvent with heat gun to yield a transparent film (delivery sheet). 200×200 nm cylindrical Fluorocur-patterned PRINT molds (Liquidia Technologies) were laminated against the delivery sheet with moderate pressure (40 psi) and then gently delaminated. The filled mold was laminated against corona-treated PET and subsequently cured in a UV chamber (λₘₐₓ=365 nm, 90 mW/cm²) for 5 min. After photocuring, the mold was removed to reveal an array of particles on PET. Particles were harvested off PET with water mechanically using a cell scraper (1 mL/48 in²). Supernatant was removed via centrifugation (15 min, 14 krpm, 4° C.). Pro-siRNA hydrogels were washed repeatedly with 10×PBS containing 0.05% PVA 2 kDa to remove the sol fraction.

Particle characterization. Scanning electron microscropy (SEM) enabled imaging of hydrogels that were dispersed on a glass slide and coated with 2 nm of Au/Pd (Hitachi S-4700).-potential measurements were conducted on 20 μg/mL particle dispersions in 1 mM KCl using a Zetasizer Nano ZS Particle Analyzer (Malvern Instruments Inc.).

Analysis of siRNA by gel electrophoresis. 2.5% agarose gel in TBE buffer was prepared with 0.5 μg/mL ethidium bromide. For studying release of siRNA from hydrogels, aliquots of particle dispersions were centrifuged (15 min, 14 krpm, 4° C.) for recovery of the supernatant at various time points and frozen. Similarly, aliquots of siRNA prodrug incubated in 10% FBS at 37° C. were taken at various time points and frozen for storage. 12 μL of sample (supernatants from particle dispersions or siRNA solutions) was mixed with 3 μL of 6× loading buffer and pipetted into the gel lanes. 70 V/cm was applied for 25 min and the gel was then imaged with ImageQuant LAS 4000 (GE). Analysis of siRNA band intensity was conducted with Image J software.

Cell culture. Luciferase-expressing HeLa cell line (HeLa/luc) was from Xenogen. HeLa/luc cells were maintained in DMEM high glucose supplemented with 10% FBS, 2 mM L-glutamine, 50 units/mL penicillin and 50 μg/mL streptomycin, 1 mM sodium pyruvate and non-essential amino acids. All media and supplements were from GIBCO except for FBS which was from Mediatech, Inc.

In vitro cell uptake analysis. HeLa/luc cells were plated in 96-well plate at 10,000/well and incubated overnight at 37° C. Cells were dosed with particles in OPTI-MEM at 37° C. (5% CO₂) for 4 h or indicated time for cell uptake studies. After incubation, cells were washed and detached by trypsinization. After centrifugation, cells were resuspended in a 0.4% trypan blue (TB) solution in Dulbecco's Phosphate Buffers Saline solution (DPBS) to quench the fluorescein fluorescence from particles associated to cell surface. Cells were then washed and resuspended in DPBS or fixed in 1% paraformaldehyde/DPBS, and analyzed by CyAn ADP flowcytometer (Dako). Cell uptake was represented as percentage of cells that were positive in fluorescein fluorescence.

In vitro cytotoxicity and luciferase expression assays. HeLa/luc cells were plated in 96-well plate at 10,000/well and incubated overnight at 37° C. Cells were dosed with particles or Lipofectamine 2000/siRNA mix in OPTI-MEM at 37° C. (5% CO₂) for 4 or 5 h, then particles were removed, and complete grow medium was added for another 48 h incubation at 37° C. Cell viability was evaluated with Promega CellTiter 96® AQᵤₑₒᵤₛ One Solution Cell Proliferation Assay, and luciferase expression level was evaluated with Promega Bright-Glo™ Luciferase Assay according to manufacturer's instructions. Light absorption or bioluminescence was measured by a SpectraMax M5 plate reader (Molecular Devices). The viability or luciferase expression of the cells exposed to PRINT particles was expressed as a percentage of that of cells grown in the absence of particles.

### 10. A. DIC Cross-Linker

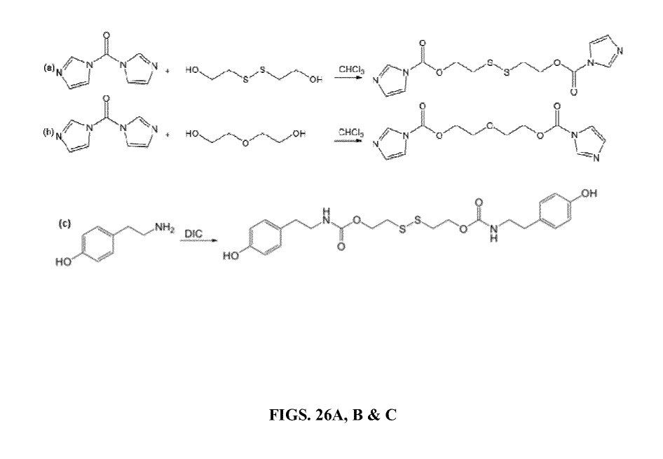

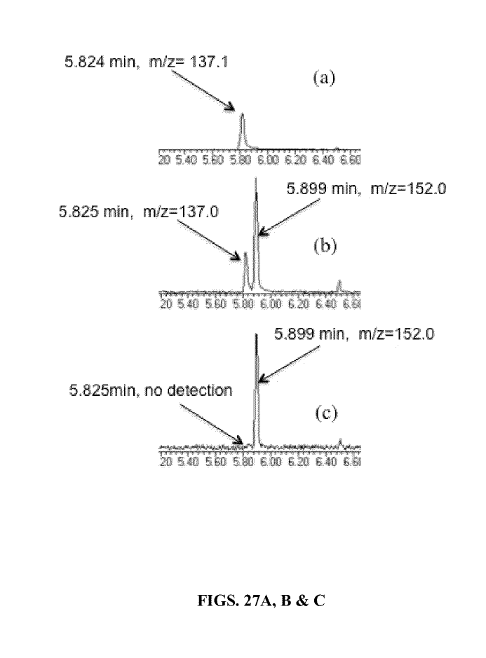

To demonstrate the ability of DIC to release the amino group in its original form after cleavage of the disulfide, we utilized tyramine as a model, a small molecule with only one amino group. Two tyramine molecules were reacted with DIC in isopropanol, which completely simulates the cross-linking conditions for protein-based particles (FIG. 26). The commercially available disulfide crosslinker DSP was used as a control in this study. The dimer products were denoted as tyramine-DIC and tyramine-DSP, respectively. Both compounds were treated with 50 mM of dithiothreitol (DTT) in phosphate buffer saline (PBS) to cleave the disulfide bond. Gas chromatography mass spectrum (GC-MS) results indicated that after cleavage of the disulfide bond, tyramine was regenerated from tyramine-DIC. No peak of tyramine was observed with tyramine-DSP (FIG. 27). In addition, ¹H-NMR and high-resolution mass spectrometry confirmed the structure of tyramine recovered after DTT treatment of tyram tyramine-DIC. (SI) Therefore, all the aforementioned results support the successful design of the novel DIC crosslinker.

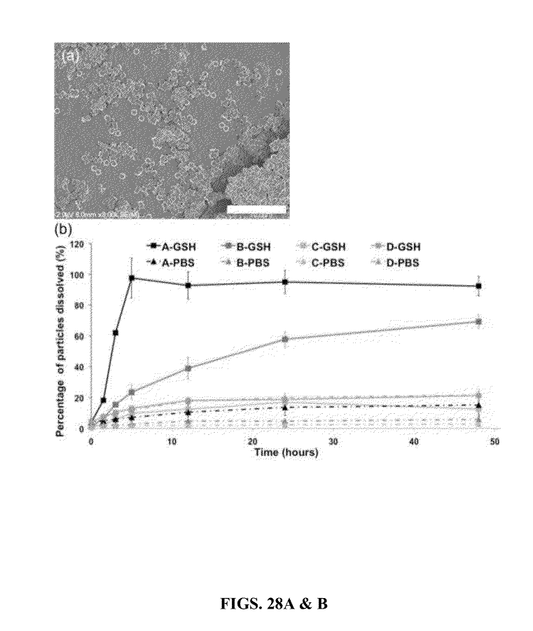

The stabilization of PRINT albumin particles in aqueous solutions was achieved by introducing DIC which reacts with the amine groups on the surface of protein molecules (FIG. 28a). Particles cross-linked with this disulfide cross-linker can take advantage of the high concentration of intracellular GSH and selectively release encapsulated cargo when they reach cyto-plasm. As a control, a non-disulfide non-degradable crosslink-er, 2,2′-oxybis(ethane-2,1-diyl) bis(1H-imidazole-1-carboxylate) (OEDIC) was also synthesized and used as a control (Scheme α(c)).

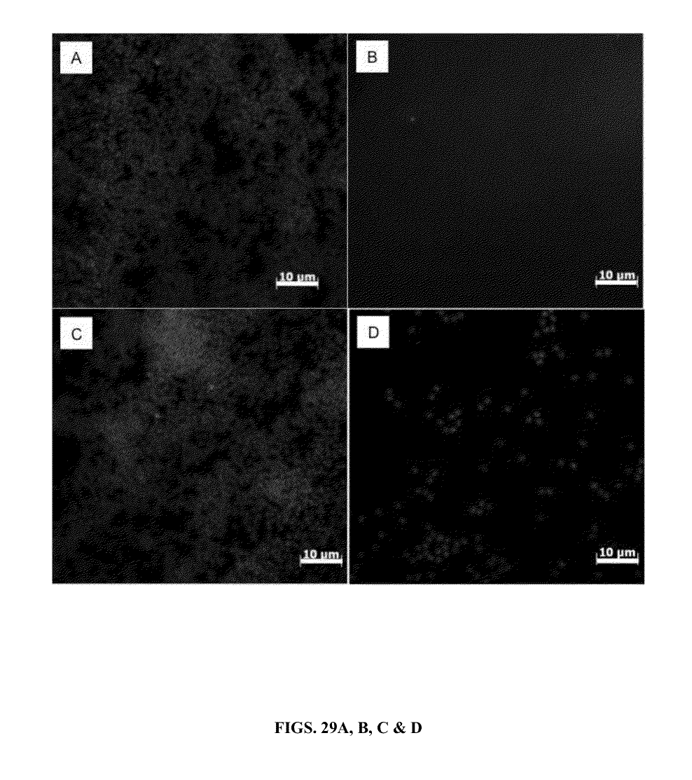

BSA particles were crosslinked with DIC and OEDIC at different cross-linker concentrations (based on a constant particle concentration) and a quantitative study of particle dissolution was performed. The GSH concentration in cytoplasm of cells ranges from 1 to 15 mM.11 In this study, PBS containing 5 mM GSH and PBS only were used to simulate intracellular and extracellular environment, respectively. In order to monitor the degradation of albumin particles, 1 wt % of BSA Alexa Fluor® 555 conjugate was incorporated into the particles and the amount of this dye-conjugated protein released from particles upon particle dissolution was measured using fluorescence spectroscopy (FIG. 28b). A plot of Alexa Fluor® 555 conjugate release versus time for particles crosslinked with DIC at 4.4 mM showed an accelerated rate of dissolution when dispersed in PBS with 5 mM GSH. The same particles dispersed in PBS only showed minimal dissolution at 48 h. Under identical conditions, particles crosslinked with OEDIC showed no noticeable difference in PBS with and without GSH. Particles crosslinked with the DIC at 6.6 mM also dissolved preferentially in PBS with GSH, but the rate was noticeably slower than particles cross-linked using 4.4 mM of DIC. When particles were crosslinked with the DIC cross-linker at 9.9 mM, very minimal dissolution of particles was observed both in PBS with GSH and PBS only during a 48-h time frame. Fluorescence microscopy was also used to further investigate the integrity of particles cross-linked with DIC and OEDIC at 4.4 mM of cross-linker concentration (FIG. 29). These data show that the particles cross-linked with the disulfide linker DIC preferentially dissolved under reducing conditions and the rate of particle dissolution can be effectively modulated by changing the cross-linker concentration used. Alternatively, the crosslinking reaction time can also be used as a parameter to fine tune the particle crosslinking densities and release profiles.

The DIC crosslinked PRINT protein particles were characterized by dynamic light scattering (DLS) and ζ-potential analyzer. The particles displayed a hydrodynamic diameter around 1 micron and a narrow polydispersisity (Table G).

TABLE G
Characterization of crosslinked BSA particles ^{a}

| | Diameter, ^{b} nm | PDI ^{c} | ζ-Potential, ^{d} mV |
|---|---|---|---|
| DIC-4.4 mM | 1201 ± 152 | 0.016 | −13.6 ± 0.5 |
| DIC-6.6 mM | 1164 ± 393 | 0.114 | −16.3 ± 1.0 |
| DIC-9.9 mM | 1069 ± 346 | 0.105 | −23.1 ± 0.4 |
| OEDIC-4.4 mM | 1069 ± 362 | 0.114 | −10.9 ± 0.3 |

^{a} The particles fabricated for dissolution study. DIC-4.4 mM: particles cross-linked with DIC at 4.4 mM. DIC-6.6 mM: particles cross-linked with DIC at 6.6 mM. DIC-9.9 mM: particles crosslinked with DIC at 9.9 mM. OEDIC-4.4 mM: particles cross-linked with OEDIC at 4.4 mM.
^{b} Hydrodynamic diameter measured by dynamic light scattering. The average hydrodynamic diameters were obtained from three measurements. The error bars are the half-width of the effective diameters.
^{c} Polydispersity index from the dynamic light scattering measurements.
^{d} ζ-potential was measured in 1 mM KCl by Zetasizer. The error bars are standard deviations from three measurements.

Because the isoelectric point of BSA is around 4.75, crosslinked BSA particles showed a slightly negative ζ-potential. Particles cross-linked at higher cross-linker concentrations showed more negative ζ-potentials due to less free amino groups on the particle surface.

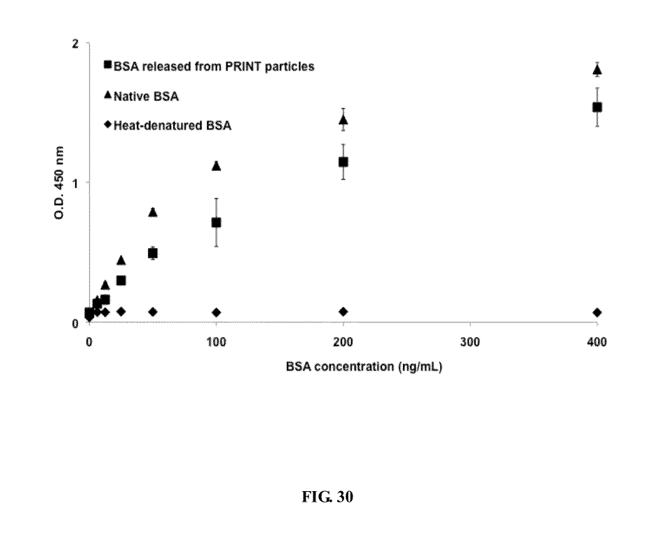

To evaluate the biological integrity of the protein after dissolution of the DIC cross-linked particles, enzyme-linked immunosorbent assays (ELISA) were performed on native BSA and BSA released from DIC-cross-linked PRINT BSA particles in PBS with 5 mM glutathione, as well as heat denatured BSA. Several concentrations were compared over the sampling range of the assays (FIG. 30). The results of the assay indicated that the albumin dissolved from the cross-linked particles (squares) had very similar absorbance in the ELISA assay as the native albumin (triangles). The denatured free protein (tilted squares) showed significantly less total absorbance compared to free protein and dissolved particles. ELISA relies upon protein-protein interactions, thus providing insight into the preservation of the protein's binding motifs. The ELISA data illustrated that antibody recognition and protein binding ability of BSA are minimally affected in the PRINT and cross-linking process for albumin.

A useful method for the fabrication of protein (BSA) particles that uses a unique cross-linker strategy effectively renders the particles transiently insoluble in aqueous solutions. This particle fabrication method built on PRINT technology platform allows for the fabrication of particles of controlled sizes and shapes. A disulfide cross-linker for the stabilization of the particles was synthesized and applied on the particles. The particles cross-linked with the cross-linker preferentially dissolved under reducing conditions and the rate of particle dissolution can be controlled by adjusting the cross-linker concentration used. The antibody recognition and pro-tein binding ability of BSA were minimally affected in the PRINT and cross-linking processes, which suggested that this method could be applied to delivery of functional proteins to the cytoplasm of cells. In addition, these precisely engineered protein particles can be used as carriers for drug and gene delivery.

### Materials.

Bovine serum albumin were from Calbiochem. Tyramine and 1′-Carbonyldiimidazole was purchased from Sigma Aldrich. BCA protein assay reagent and DSP (Dithiobis[succinimidyl propionate]) were from Thermo Scientific. Alexa fluor 555® labeled Bovine serum albumin was purchased from invitrogen. Lactose assay kit was purchased from Abcam. Bovine albumin ELISA quantitation set was purchased from Bethyl Laboratories, Inc. α-D-Lactose, glycerol, 2-hydroxyethyl disulfide and bis(2-hydroxyethyl)ether were purchased from Acros.

### Crosslinker Synthesis.

A solution of 2-hydroxyethyl disulfide (1 g, 6.48 mmol) in chloroform (50 mL) was added dropwise to a solution of 1,1′-Carbonyldiimidazole (10 g, 61.67 mmol) in chloroform (300 mL) under reflux (FIG. S3a). The reaction mixture was stirred for 24 hours. The mixture was washed with cold water three times and the organic layer was dried with magnesium sulfate, filtered, concentrated and purified by column chromatography (EtOAc/chloroform=95:5) to give DIC (0.85 g) as clear oil, which turned to white solid upon cooling. The reaction of Bis(2-hydroxyethyl)ether (0.69 g, 6.48 mmol) with 1,1′-Carbonyldiimidazole (10 g, 61.67 mmol) gave OEDIC (0.73 g) as clear oil, which turned to white solid upon cooling. The synthesis and purification followed procedures described above for DIC.

### Preparation of Protein Based Particles.

The bovine serum albumin (BSA) PRINT particles were derived from a mixture composed of 37.5 wt % of BSA, 37.5 wt % of D-lactose and 25 wt % of glycerol. A 7.8 wt % solution of this mixture in water was prepared and then cast a film onto a poly(ethylene terephthalate) (PET) sheet. Water was removed with a heat gun moving back and forth. The film should be transparent and was laminated onto a piece of fluorocur patterned mold (4×4 inch, cylindrical, d=1 μm, h=1 μm), forming a sandwich structure with the film in the middle. The mold was delaminated by passing the mold and the PET through a heated laminator with a temperature of 60° C. on the top roller and a pressure of 80 psi between the rollers. The filled mold was re-laminated onto a sheet of plasdone covered PET. The laminated mold and PET were passed through the heated laminator again. After the particle cooled down, the mold and the PET were separated gently and all the PRINT particles were transferred from the mold to the plastone film. The particles were harvested from the PET by dissolving plastone with isopropanol. The harvested particles were washed with isopropanol for three times by centrifugation to remove plastone. The particles were finally dispersed in isopropanol and the particle concentration was determined by Thermal Gravimetric Analysis (TGA) (TA Q5000).

### Preparation of Alexa Fluor 555® Labeled BSA Particles.

The Alexa fluor 555® labeled BSA particles were derived from a mixture composed of 37.0 wt % of BSA, 37.0 wt % of lactose, 25.0 wt % of glycerol and 1.0 wt % of Alexa fluoro 555 labeled BSA.

### Quantification of BSA and Lactose in Particles Prior to the Crosslinking Reaction:

Particles were dispersed in water. A BCA assay (Thermo scientific) was used to quantify the amount of BSA in the solution and a lactose quantification kit (Abcam) was used to quantify the amount of lactose in the solution. Each assay was done in duplicate and three independent samples were measured.

### Particle Cross-Linking Reaction.

Based on the TGA results, an appropriate amount of isopropanol was added to the particle dispersion to achieve a particle concentration of 1 mg/mL. To 850 μL of particle dispersion, 1.275 mg of DIC was added. The resulting dispersion was shaken on a vortex machine for 24 h at 40° C. The reaction was terminated by centrifuging particles down for 3 minutes, followed by removal of the supernatant containing the cross-linker and adding 850 μL of isopropanol. The particles were washed three times with isopropanol by centrifugation to remove the excess cross-linkers and then resuspended in water.

### Physical Characterization of the PRINT Protein Particles.

The PRINT particles were imaged by a scanning electron microscopy (Hitachi modelS-4700) and the hydrodynamic diameters of the PRINT particles were measured by dynamic light scattering (Brookhaven Instruments Inc., 90Plus). For zeta potential measurements, the particles were dispersed in 1 mM potassium chloride at a concentration of 20 μg/ml and tested by a Zetasizer Nano Analyzer (Malvern Instruments Inc., Nano Zetasizer).

### Dissolution Studies.

Bovine serum (BSA), Alexa Fluor® 555 conjugate was incorporated into the particles and the release of this dye-conjugated protein was used to characterize the dissolution rate of the particles. Typically, particles were fabricated from a mixture of 37 wt % of BSA, 1 wt % of albumin from bovine serum (BSA), Alexa Fluor® 555 conjugate, 37 wt % of D-lactose and 25 wt % of glycerol. The particles were crosslinked and then resuspended in water to achieve a particle concentration of 1.33 mg/mL following the procedures described above. To each mini dialysis unit (purchased from Fisher Scientific, MWCO 20K), 75 μL of particle solution was added. Typically, 24 units were dialyzed against 1 L of Phosphate Buffers Saline solution (PBS) containing 5 mM glutathione with a magnetic bar stiffing gently at the bottom of the beaker. Another 24 units were dialyzed against 1 L of PBS buffer without glutathione as controls. The dialysis process was carried out in a 37° C. incubator. At different time points (0 h, 1.5 h, 3 h, 5 h, 12 h, 24 h, 48 h), one unit was withdrawn from each bath. The particle solution was recovered from the units and each unit was washed with 75 μL of PBS. The wash was combined with recovered particle solution and appropriate amount of PBS was added to achieve a total mass of 200 mg. The solution was centrifuged at 14000 rpm for 10 min. The supernatant was measured for fluorescence (excitation 545 nm, emission 575 nm) by a SpectraMax M5 plate reader (Molecular Devices). The fluorescence from PBS was used as background and the fluorescence from un-cross-linked particles (0.5 mg/mL in PBS) was used as a 100% control.

### BSA ELISA Characterization.

The BSA particles were crosslinked at 4.4 mM of DIC for 24 h at 40° C. and incubated in PBS containing 5 mM GSH for 5 h. The solution was then dialyzed against water for 2 h to remove GSH. The concentration of BSA was quantified by BCA assay and standard sandwich ELISA assays for BSA (Bethyl Laboratories, Montgomery, Tex.) were conducted following the protocol provided by the vendor. Absorbance was measured with a SpectraMax M5 plate reader (Molecular Devices) at 450 nm.

### A Fully Reversible Disulfide Crosslinker.

Tyramine (0.24 g, 1.75 mmol) was added to a solution of DIC (0.12 g, 0.35 mmol) in isopropanol (15 mL). The reaction mixture was stirred for 24 h at 40° C. The mixture was concentrated and purified by column chromatography (EtOAc) to give tyramine-DIC (0.10 g, 99%) as light yellow solid. Tyramine (0.24 g, 1.75 mmol) was added to a solution of DSP (0.14 g, 0.35 mmol) in DMF (4 mL). The reaction mixture was stirred for 24 h at 40° C. The reaction was stopped by adding water (15 mL) to the reaction mixture. Then the product was filtered and washed with water (10 mL) three times. The product tyramine-DSP (light yellow solid) was then dried and weighed (0.11 g). The products tyramine-DSP and tyramine-DIC were added to dithiothreitol solution (50 mM, PBS) at 37° C. and stirred for 24 h. Then the solutions were lyophilized Isopropanol (1 mL) was added to the powder acquired and bath sonicated for 15 min. The supernatants from the solutions were collected and analyzed by gas chromatography-mass spectrometry (Alilent Technologies 5975 series MSD, 7820A GC system) and untreated tyramine was used as standard.

Tyramine generated from tyramine-DIC was purified through thin layer chromatography (TLC) (EtOAc 90%, methanol 10%). ¹H NMR (bruker Avance 400WB) and mass spectrometry (Agilent technologies 6210 LC-TOF) were used to confirm the structure of the compound.

### Compound Characterization

### Dithio-bis(ethyl 1H-imidazole-1-carboxylate) DIC

¹H NMR (400 MHz, CDCl₃) δ 8.18 (s, 2H), 7.45 (s, 2H), 7.11 (s, 2H), 4.71 (t, J=6.8 Hz, 4H), 3.11 (t, J=6.4 Hz, 4H)

¹³C NMR (150 MHz, CDCl₃) δ 36.3, 65.6, 117.2, 130.8, 137.1, 148.4

MS (LC-TOF) m/z 365.0346 (M+Na)+

### 2,2′-Oxybis(ethane-2,1-diyl) bis(1H-imidazole-1-carboxylate) OEDIC

¹H NMR (600 MHz, CDCl₃) δ 8.17 (s, 2H), 7.43 (s, 2H), 7.10 (s, 2H), 4.61 (t, J=6.6 Hz, 4H), 3.89 (t, J=7.2 Hz, 4H)

¹³C NMR (150 MHz, CDCl₃) δ 66.5, 68.4, 115.9, 130.5, 137.0, 148.4

MS (LC-TOF) m/z 317.0859 (M+Na)+

### Tyramine-DIC

¹H NMR (400 MHz, actone-D6) δ 7.07 (d, J=8.4 Hz, 4H), 6.78 (d, J=8 Hz, 4H), 4.28 (t, J=7.6 Hz, 4H), 3.33 (q, J=6.4 Hz, 4H), 2.98 (t, J=6.4 Hz, 4H), 2.74 (t, J=7.2 Hz, 4H)

¹³C NMR (100 MHz, actone-D6) δ 155.8, 130.1, 129.6, 115.2, 115.1, 62.0, 42.6, 37.8, 35.1

MS (LC-TOF) m/z 503.1274 (M+Na)+

### Tyramine-DSP

¹H NMR (400 MHz, actone-D6) δ 7.08 (d, J=8.4 Hz, 4H), 6.78 (d, J=8.8 Hz, 4H), 3.41 (q, J=7.6 Hz, 4H), 2.98 (t, J=7.2 Hz, 4H), 2.73 (t, J=7.2 Hz, 4H), 2.57 (t, J=6.8 Hz, 4H)

¹³C NMR (100 MHz, actone-D6) δ 170.4, 156.0, 130.0, 129.6, 115.3, 41.0, 35.4, 34.7, 34.4

MS (LC-TOF) m/z 471.1388 (M+Na)+

### Tyramine Generated from Tyramine-DIC

¹H NMR (400 MHz, MeOD) δ 7.02 (d, J=4.2 Hz, 2H), 6.71 (d, J=4.2 Hz, 2H), 2.82 (t, J=7.2 Hz, 2H), 2.65 (t, J=7.2 Hz, 2H)

MS (LC-TOF) m/z 138.0916 (M+H)+

### Tyramine Standard

¹H NMR (400 MHz, MeOD) δ 7.02 (d, J=4.2 Hz, 2H), 6.71 (d, J=4.2 Hz, 2H), 2.81 (t, J=7.2 Hz, 2H), 2.65 (t, J=7.2 Hz, 2H)

MS (LC-TOF) m/z 138.0914 (M+H)+

### 11. A. Fabrication of RNA Replicon Incorporated PRINT Particle

Protein was chosen as the matrices for RNA replicon delivery based on the fact that both RNA replicon and protein are highly hydrophilic and dissolve readily in aqueous solutions in which RNA replicon and protein can be evenly mixed together and subsequently molded into particles utilizing PRINT technology. Serum albumin was chosen as the protein for the study for two reasons. Serum albumin is one of the most readily available proteins and has demonstrated tremendous success as a small molecule delivery matrix in the clinics (Hawkins M J, Soon-Shiong P, Desai N (2008) Protein nanoparticles as drug carriers in clinical medicine. Advanced Drug Delivery Reviews 60:876-885). In particular, bovine serum albumin (BSA) was used due to its accessibility in RNAse-free grade and its cost effectiveness for our proof-of-concept study. Based on previous studies, dendritic cells, the target cell for RNA replicon based vaccines, preferentially take up micron sized particles (Bachmann M F, Jennings G T (2010) Vaccine delivery: a matter of size, geometry, kinetics and molecular patterns NATURE REVIEWS IMMUNOLOGY 10:787-796; O'Hagan D T, Singh M, Ulmer J B (2006) Microparticle-based technologies for vaccines Methods 40:10-19). In this study, cylindrical particles with both diameter and height as 1 μm were fabricated. We have demonstrated that protein particles can be fabricated by mixing protein with lactose and glycerol to form the preparticle material that flows into the cavities when heated. Based on our previous success, RNA replicon was incorporated into the particle by mixing the cargo with BSA, lactose and glycerol (FIG. 31).

Briefly, a film of protein, lactose, glycerol and RNA replicon mixture is cast on a polyethylene terephthatlate (PET) sheet. Water is removed and the film is heated in contact with a PRINT mold (mold No.: MMM-262-090A, MMM-369-070) while going through a pressured nip where the mixture is heated and melts into the cavities. Due to the unique nonwetting nature of PRINT mold, the cavities are filled without forming a “flash” layer between the particles. The particles can then be transferred to a sacrificial adhesive layer, which can be dissolved to release the PRINT particles. Following the aforementioned PRINT process, RNA replicon incorporated cylindrical BSA particles with both diameter and height as 1 μm were fabricated with a preparticle composition containing 37 wt % of BSA, 37 wt % of α-D-lactose, 25 wt % of glycerol and 1 wt % of RNA replicon.

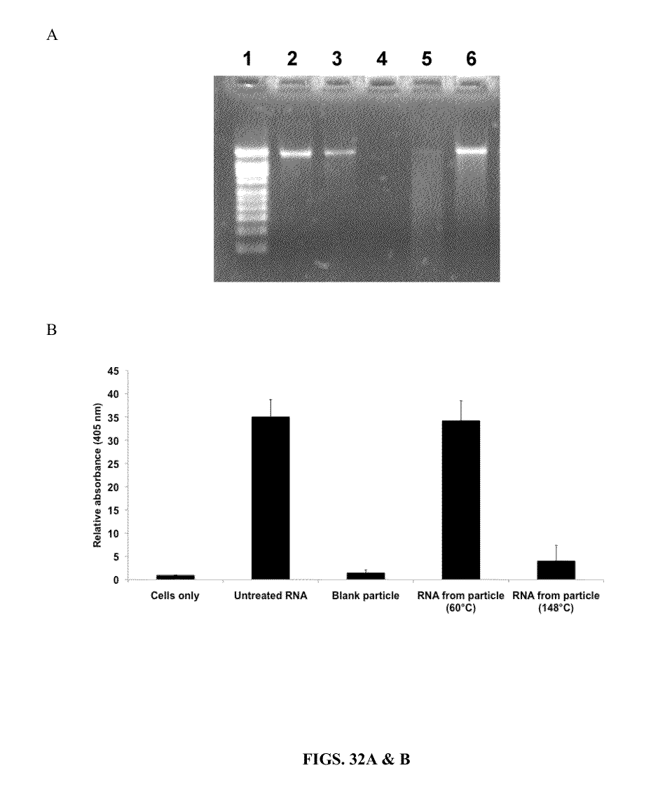

RNA replicon is a single stranded RNA with low stability. Maintaining its integrity in the process of PRINT particle fabrication is essential. We studied the influence of temperature used for particle fabrication on the biological activity of RNA replicon. A model RNA replicon encoding chloramphenicol acetyl transferase (CAT) was chosen in this study because CAT is a bacterial enzyme and exogenous for mammalian cells and the assays to quantify CAT activity has been well established. Typically, 1 wt % of CAT RNA replicon was charged into cylindrical albumin particles (d=1 μm, h=1 μm) by using two temperatures on the heated laminator roller: 60° C. vs. 148° C. The particles were dissolved in phosphate buffered saline (PBS) and followed by extraction of RNA replicon from the BSA-RNA replicon mixture. It should be noted that the particles used in this experiment did not involve any crosslinking and were readily soluble in water. As a control, the RNA extraction procedure was also performed on the blank particles to rule out any existence of RNA in the BSA used for particle fabrication. The integrity of the extracted RNA replicon was first evaluated using agarose gel electrophoresis, as shown in FIG. 32a. Electrophoresis analysis showed that RNA replicon encapsulated at 60° C. displayed a tight band at the same position as the untreated RNA replicon. However, when particles were fabricated at 148° C., only some smears, which were speculated as RNA replicon degradation products, were observed. The integrity of extracted RNA replicon was further accessed by a CAT ELISA assay after RNA replicon was transfected into Vero cells, a kidney epithelial cell line developed from an African green monkey, using a TransIT®-mRNA transfection reagent (TransIT). The CAT ELISA assay quantifies the amount of CAT protein generated by the cells after RNA replicon transfection. Results showed that RNA replicon encapsulated at 148° C. showed very minimal biological activity and RNA extracted from particles fabricated at 60° C. produced similar protein expression levels to untreated RNA replicon control (FIG. 32b). The results showed that RNA replicon is very sensitive to the temperature used for particle fabrication and lower temperature is preferred to preserve RNA replicon activity.

After the harvest and purification steps using isopropanol, the particles fabricated at 60° C. were determined to contain 1.5±0.1 wt % of RNA replicon after purification step in the final harvested particle composition (Table H).

TABLE H
Particle composition

| | Charged | Final |
|---|---|---|
| | Composition ^{a} | Composition ^{b} |
| | (wt %) | (wt %) |
| BSA | 37.0 | 81.5 ± 0.2 |
| Lactose | 37.0 | 10.3 ± 3.1 |
| Glycerol | 25.0 | — |
| RNA Replicon | 1.0 | 1.5 ± 0.1 |

^{a} The weight percentage of components charged into the preparticle solution that was then drawn into a film on the PET sheet.
^{b} Final particle composition after harvest and purification step. The errors stand for standard deviation calculated from three experiments.

### B. Reversible Disulfide Crosslinker for Protein-Based RNA Replicon Particle Stabilization

In order to utilize protein-based particles as carriers for drug delivery, they are usually stabilized with reversible crosslinkers that can be cleaved under certain physiological stimuli, such as acidic or reducing environment (Yu M, Ng B C, Rome L H, Tolbert S H, Monbouquette H G (2008) Reversible pH lability of cross-linked vault nanocapsules. Nano Lett 8:3510-3515; Jia Z, Liu J, Boyer C, Davis T P, Bulmus V (2009) Functional Disulfide-Stabilized Polymer-Protein Particles Biomacromolecules 10: 3253-3258). However, the linkers are not necessarily “traceless.” The site of action for RNA replicon is cytoplasm, which is known for its high concentration of reduced glutathione (GSH) compared to extracellular environment (GSH intracellular concentration between 5 mM and 15 mM) (Saito G, Swanson J A, Lee K (2003) Drug delivery strategy utilizing conjugation via reversible disulfide linkages: role and site of cellular reducing activities. Advanced Drug Delivery Reviews 55:199-215).

Lomant's reagent, dithiobis[succinimidyl propionate] (DSP), is commercially available and widely used in protein crosslinking between lysine residues. DSP is basically a disulfide based di-N-hydroxysuccinimide (NHS) ester. Our initial studies using DSP to crosslink BSA-based RNA replicon containing particles did not show any biological activities in vitro (data not shown). Firstly, the di-NHS ester DSP is highly reactive towards lysine residues on BSA. It is very difficult to control the crosslinking density of BSA particles, which is essential to achieve desired particle release profile of RNA replicon in the cytoplasm. Secondly, NHS esters are known to react with not only amines but also hydroxyl groups. Each nucleotide in RNA has a free hydroxyl group at the 2′ position of the ribose sugar, which is susceptible to reactive NHS esters. In addition, the free amine groups on nucleobases are also likely to react with NHS ester based crosslinkers. Thirdly, even though DSP is advertised as a reversible crosslinker, it is not a “truly” reversible crosslinker and leaves molecular pedants after disulfide cleavage which may render the released protein be regarded as foreign antigens by the immune system and trigger dangerous health effects.

Due to all the aforementioned reasons, we used dithio-bis(ethyl 1H-imidazole-1-carboxylate) (DIC) as the crosslinker to stabilize the protein particles in aqueous solutions. This crosslinker was developed and, compared to DSP, has several advantages for protein particle stabilization for RNA replicon delivery. Imidazoles as the leaving groups enhanced the reaction selectivity for amines over hydroxyl groups compared to NHS esters, which is essential to maintain the integrity of RNA replicon in the crosslinking procedure. Furthermore, DIC is a “traceless” reversible crosslinker, which does not leave any molecular pendants after disulfide. A truly reversible chemistry has many benefits for the delivery of RNA replicon. It releases the amino groups in its original form and avoids the unknown immune response towards novel antigens. Additionally, even if the amine functionalities on nucleobases were crosslinked by DIC, due to the fully reversible nature of the crosslinker, the nucleobases should revert to its original state upon disulfide cleavage if DIC is used.

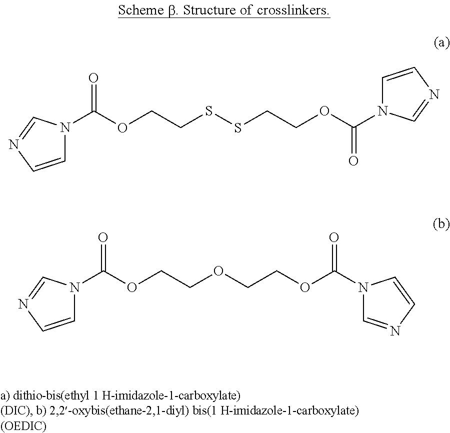 [SMILES: O=C(OCCSSCCOC(=O)n1ccnc1)n1ccnc1 ; O=C(OCCOCCOC(=O)n1ccnc1)n1ccnc1]

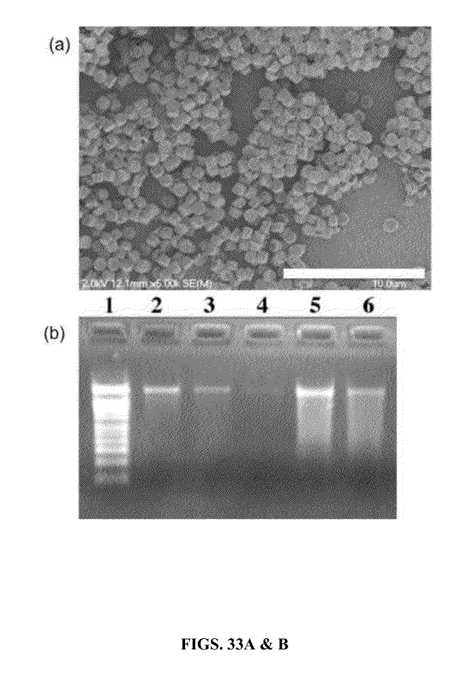

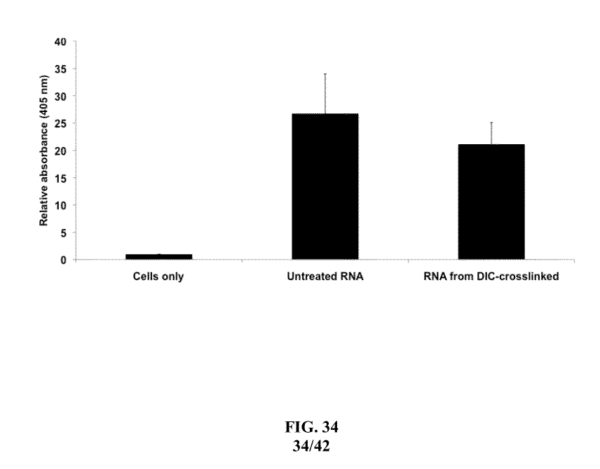

The stabilization of PRINT albumin particles in aqueous solutions was achieved by introducing DIC as the crosslinker (FIG. 33a). To assess the integrity of RNA replicon after crosslinking reaction, particles were dissolved in 10 mM DTT solution and the RNA replicon extracted from the mixture was evaluated by agarose gel electrophoresis (FIG. 33b). The agarose gel results showed that after crosslinking reaction the RNA replicon inside protein particles was minimally affected. RNA replicon integrity was also confirmed by induced expression of CAT protein after transfection to Vero cells as described in Section 1 (FIG. 34).

### C. Delivery of CAT RNA Replicon Particles

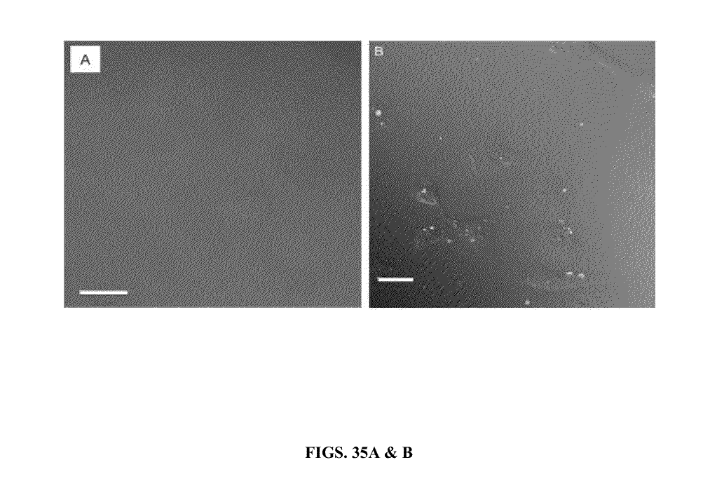

Due to the isoelectric point of BSA (pI=4.75), the crosslinked BSA particles with RNA replicons are negatively charged (ζ potential=−15.4±1.0 mV). Based on the previous studies from our group and other groups, cells generally preferentially internalize positively charged particles through a non-specific electrostatic interactions between the positively charged particles and the negatively charged cell membrane. Our confocal microscopy studies confirmed that negatively charged BSA particles did not show significant cell uptake (FIG. 35). In order to introduce positive charges to BSA particle surface to enhance cell uptake, a certain amount of TransIT was mixed with BSA particles. The introduction of TransIT is also expected to enhance the endosomal escape of BSA particles, which is the major roadblock for RNA replicon delivery. The crosslinked particles mixed with TransIT achieved a positively charged particle surface (ζ potential=+0.8±0.3 mV) and were subsequently incubated with Vero cells for 4 h at 37° C. and the non-internalized particles were removed (Table I).

TABLE I
Characterization of crosslinked BSA
particles with and without TransIT ^{a}

| | Diameter, nm | PDI | ζ-Potential, mV |
|---|---|---|---|
| Without TransIT | 1214 ± 483 | 0.159 | −15.4 ± 1.0 |
| With TransIT | 1179 ± 721 | 0.374 | +0.8 ± 0.3 |

^{a} The particles charged with 1 wt % of CAT RNA replicon. The particles (2 μg) were added into 100 μL (Opti-MEM ® I Reduced-Serum Medium), and then 2 μL of boost and 1 μL of TransIT were added subsequently. The reaction went for 5 min before measurements were taken.

Confocal microscopy studies showed that particles coated with TransIT were internalized by Vero cells (FIG. 35).

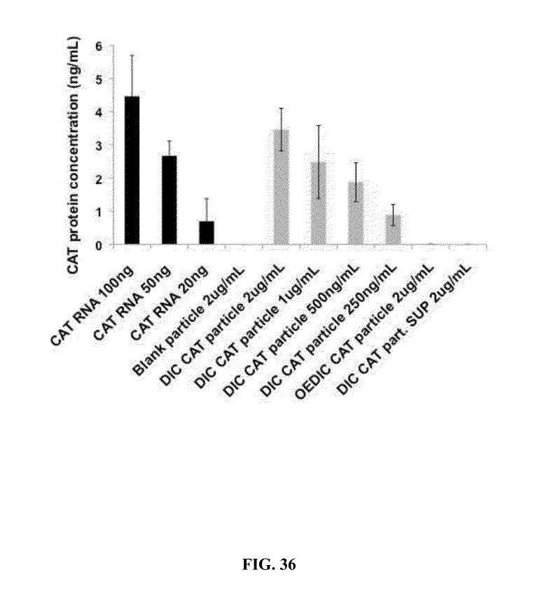

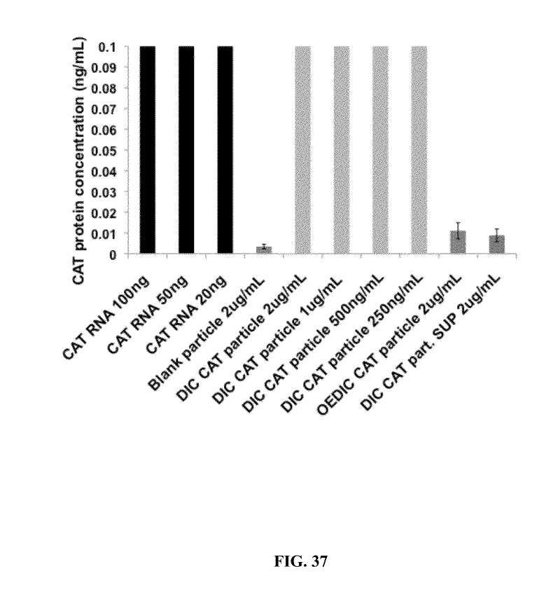

The cells were further incubated for another 48 h at 37° C. to allow CAT protein to express. The CAT protein generated via delivery of PRINT particles was comparable to the same amount of RNA replicon directly delivered by TransIT (FIG. 36). As a negative control, blank particles with no RNA replicon encapsulated didn't induce any protein expression (FIGS. 36 & 37). However, it was possible that the protein expression might be induced by RNA replicon that passively released from protein particles through diffusion and transfected by TransIT.

To investigate this possibility, DIC-crosslinked BSA particles containing CAT RNA replicon were incubated in PBS for 4 h at 37° C., which is the dosing condition for the RNA replicon delivery studies, and pelleted down through centrifugation. The supernatant was dosed to cells with TransIT and no protein expression was observed. This result confirmed that RNA replicon was physically entrapped in the BSA particles and was released in the cytoplasm of Vero cells, where CAT protein was expressed.

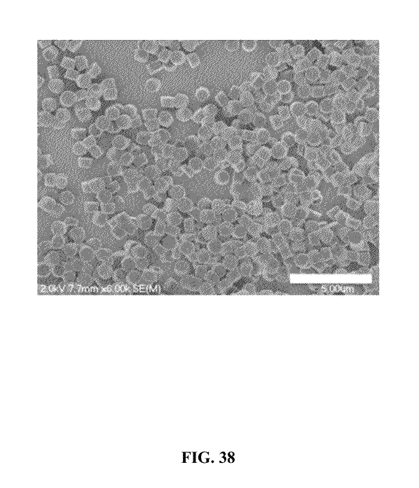

To study the necessity of a disulfide crosslinker in the delivery of RNA replicon via protein particles, particles crosslinked with a non-degradable linker 2,2′-oxybis(ethane-2,1-diyl) bis(1H-imidazole-1-carboxylate) (OEDIC) under the same reaction condition as DIC was also investigated (FIG. 38). Because both DIC and OEDIC have imidazole as the leaving group and the concentrations of crosslinkers and the reaction time are the same, we expect BSA particles crosslinked with DIC and OEDIC have similar crosslinking density. It was observed that very minimal protein was expressed with particles crosslinked by OEDIC compared to particles crosslinked by the disulfide crosslinker DIC. This result demonstrated that 1) a disulfide linker is important to achieve cytoplasm release of RNA replicon; 2) the intracellular protease were not responsible for the BSA particle degradation and RNA replicon release at least during the 48-h time frame of our assay; and 3) passive release of RNA replicon through diffusion contributed little to the observed biological activity. Therefore, the disulfide linker DIC not only stabilizes particles in the process of delivery, but also efficiently releases RNA replicon at the ultimate site of action.

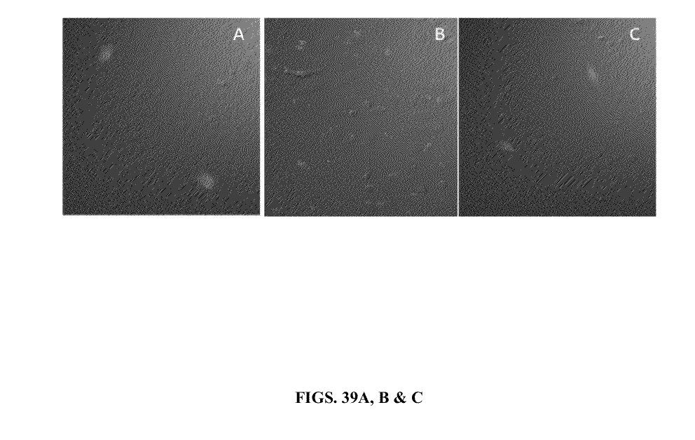

Analysis using confocal microscopy was carried out to visually confirm the generation of CAT protein. Vero cells treated with BSA particles containing RNA replicon were further treated with a primary antibody that binds specifically to CAT protein, and further treated with dye-labeled secondary antibody. Compared to untransfected cells, cells transfected with DIC-crosslinked RNA replicon-containing particles showed intense fluorescence, indicating for high levels of expression of CAT proteins in those cells (FIG. 39). Blank particles as a negative control showed no trace of CAT protein.

### D. Delivery of Luciferase RNA Replicon and GFP RNA Replicon

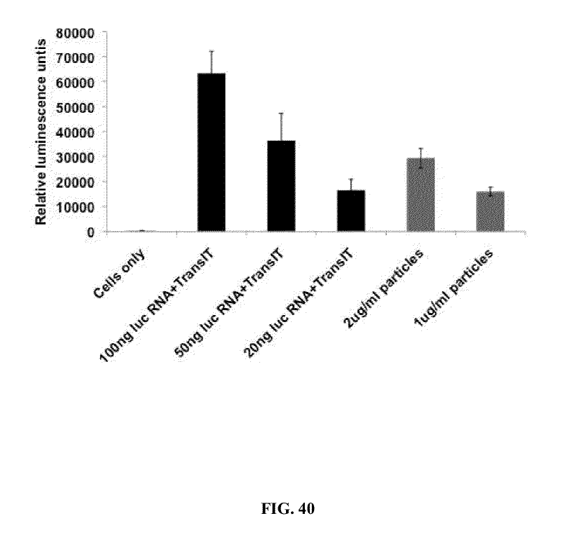

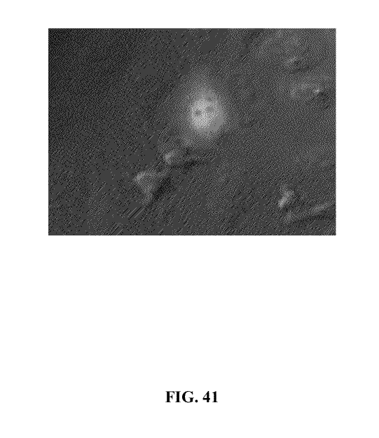

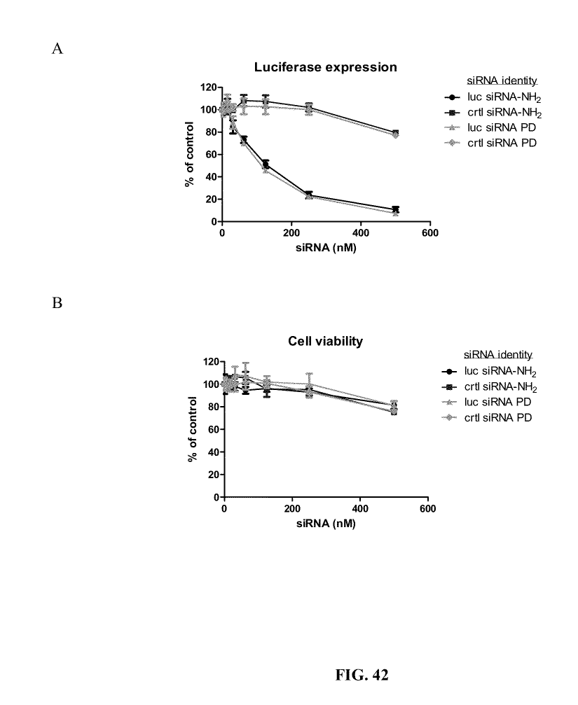

To show that RNA replicons encoding different proteins can be encapsulated and delivered within the same PRINT protein particle, RNA replicons encoding Luciferase and GFP were incorporated into BSA-based particles and delivered to Vero cells with TransIT. Both Luciferase and GFP are exogenous for Vero cells and their detection or quantification methods have been well established. Luciferase is an enzyme that catalyzes luminescent reactions and has been widely used in non-invasive bioluminescence imaging research. RNA replicons endcoding GFP protein was chosen due to the fact that GFP protein exhibits bright green fluorescence when exposed to ultraviolet blue light, which can be easily visualized with fluorescent microscope. The Luciferase protein generated via delivery of PRINT particles was comparable to the same amount of RNA replicon directly delivered by TransIT (FIG. 40). The green fluorescence generated by PRINT particles containing GFP RNA replicons was observed with fluorescent microscope, indicating successful delivery of GFP RNA replicon to the Vero cells (FIG. 41). These results suggested that PRINT is a platform for molding therapeutics with straightforward incorporation of different cargos and can work in a “plug and play” manner, which is particularly important for development of new vaccines for epidemic diseases.

A useful method for the delivery of RNA replicon via protein (BSA) particles was demonstrated. This particle fabrication method, built on PRINT technology platform, not only allows for the fabrication of particles of controlled sizes and shapes, but also was gentle and RNA replicon could be encapsulated in the particles without abolishing their biological activities. A disulfide crosslinker was used to stabilize the particles in aqueous solutions. The disulfide crosslinker was demonstrated to be RNA-friendly and stabilized the particles without affecting the biological performance of RNA replicons. By coating the particles with TransIT, the particles were delivered to Vero cells and CAT protein was expressed via delivery of PRINT particles. The reversible disulfide linker was demonstrated to play a vital role in the successful delivery of RNA replicon. RNA replicons encoding different proteins including Luciferase and GFP were incorporated into the PRINT particles and delivered using the same strategy. The PRINT technology allows for fabrication of protein particles with the ability to encapsulate therapeutics in an easy and gentle way, showing the first non-viral delivery for RNA replicon and great promise as a highly tunable drug delivery system.

### Materials and Methods

Materials. Bovine serum albumin and Fluorsave™ reagent were from Calbiochem. Tyramine and 1′-Carbonyldiimidazole was purchased from Sigma Aldrich. BCA protein assay reagent was from Thermo Scientific. Alexa fluor 555® labeled Bovine serum albumin, Alexa fluor 488® labeled Bovine serum albumin, Alexa Fluor® 546 goat anti-rabbit IgG (H+L) and Quant-iT™ RNA assay kit were purchased from invitrogen. Lactose assay kit and Anti-Chloramphenicol Acetyltransferase antibody were purchased from Abcam. TransIT®-mRNA transfection kit was purchased from Minis. Bovine albumin ELISA quantitation set was purchased from Bethyl Laboratories, Inc. α-D-Lactose, glycerol, 2-hydroxyethyl disulfide and bis(2-hydroxyethyl)ether were purchased from Acros.

Cells and culture: Vero cells were maintained at 37° C. in an atmosphere containing 5% CO₂. The cells were grown in Minimum Essential Medium (MEM; Invitrogen, Carlsbad, Calif.) supplemented with 5% fetal bovine serum (FBS, HyClone, Logan, Utah), MEM non-essential amino acid solution (Invitrogen) and antibiotic-antimycotic (Invitrogen).

CAT RNA replicon construction and preparation: Capped replicon RNAs were in vitro transcribed using a T7 RiboMax kit (Promega, Madison Wis.) following the manufacturer's instructions, supplemented with 7.5 mM CAP analog (Promega), from NotI linearized replicon plasmid. RNAs were purified using RNEasy purification columns (Qiagen, Valencia, Calif.) following the manufacturer's instructions.

Preparation of RNA replicon loaded BSA-based particles. The bovine serum albumin (BSA) PRINT particles were derived from a mixture composed of 37.0 wt % of BSA, 37.0 wt % of lactose, 25.0 wt % of glycerol and 1.0 wt % of RNA replicon (CAT, Luciferase or GFP). A 7.8 wt % solution of this mixture in water was prepared and then cast a film onto a poly(ethylene terephthalate) (PET) sheet. Water was removed with a heat gun moving back and forth. The film was laminated onto a piece of PRINT mold (2×4 inch, cylindrical, d=1 μm, h=1 μm), forming a sandwich structure with the film in the middle. The mold was delaminated by passing the mold and the PET through a heated laminator with a temperature of 60° C. on the top roller and a pressure of 80 psi between the rollers. The filled mold was relaminated onto a sheet of plastone covered PET. The laminated mold and PET were passed through the heated laminator again. After the particle cooled down, the mold was removed gently and all the PRINT particles were transferred from the mold to the plastone-covered PET. The particles were harvested from the PET by dissolving plastone with isopropanol. The harvested particles were washed with isopropanol for three times by centrifugation to remove plastone. The particles were finally dispersed in isoprapanol and the particle concentration was determined by Thermal Gravimetric Analysis (TGA) (TA Q5000).

Preparation of RNA replicon loaded Alexa fluor 488® labeled BSA particles. RNA replicon loaded Alexa fluor 488® labeled BSA particles were derived from a mixture composed of 36.7 wt % of BSA, 37.0 wt % of lactose, 25.0 wt % of glycerol, 1.0 wt % of RNA replicon and 0.3 wt % of Alexa-fluoro-488 labeled BSA.

Quantification of BSA, lactose and RNA replicon in particles prior to crosslinking reaction: Particles were dissolved in water. BCA assay (Thermo scientific) was used to quantify the amount of BSA in the solution and a lactose quantification kit (Abcam) was used to quantify the amount of lactose in the solution. Each assay was done in duplicate and three independent samples were measured. Quant-iT™ RNA assay kit (Invitrogen) was used to quantify the amount of RNA in the solution. The assay was done in duplicate and three independent samples were measured.

Particle crosslinking reaction. Based on the TGA result, an appropriate amount of isopropanol was added to the particle dispersion to achieve a particle concentration of 1 mg/mL. To 8504 of particle dispersion, 1.275 mg of DIC was added. The resulting dispersion was shaken on a vortex machine for 24 h at 40° C. The reaction was terminated by centrifuging particles down and removing the supernatant containing the crosslinker. The particles were washed three times with isopropanol by centrifugation to remove the excess crosslinkers and stored in −80° C. before other assays.

Physical Characterization of the PRINT Protein Particles. The PRINT particles were incubated in PBS for 4 h. The particles were then deposited on glass slide, coated with palladium/gold and imaged by a scanning electron microscopy (Hitachi modelS-4700). The hydrodynamic diameters of the PRINT particles were measured by dynamic light scattering (Brookhaven Instruments Inc., 90Plus). For zeta potential measurements, the particles were dispersed in 1 mM potassium chloride at a concentration of 20 μg/ml and tested by a Zetasizer Nano Analyzer (Malvern Instruments Inc., Nano Zetasizer).

RNA replicon extraction from un-crosslinked particles and DIC-crosslinked particles. For particles prior to crosslinking, 50 μL of PBS was added to dissolve 0.15 mg particles. For crosslinked particles, 50 μL of PBS containing 10 mM DTT was added to dissolve 0.15 mg particles. A Qiazol-chloroform extraction procedure was used to extract RNA replicon from the RNA replicon-BSA mixture. The RNA pellet acquired was dissolved in 20 μL of DEPC-treated water.

Agarose gel electrophoresis. Agarose gel was prepared by dissolving agrasose in 1× NorthernMax®-Gly gel preparation and running buffer (Ambion) at 1 wt %. Typically, 5 μL of sample was mixed with 5 μL of water and 10 μL of NorthernMax®-Gly load dye (Ambion) and heated at 50° C. for 10 min before loading onto the gel. The gel was then run in 1× NorthernMax®-Gly gel preparation and running buffer (Ambion) at 70 V for 35 min before being imaged by a GE ImageQuant LAS 4000 biomolecular imager.

Evaluation of RNA replicon activity through CAT expression. Typically, 2×10⁴ Vero cells were plated into 24 well tissue cultured treated plates 18-24 h prior to assay. Vero cells were transfected with CAT RNA replicon utilizing the TransIT® mRNA transfection kit (Mirus Bio, Madison, Wis.) following the manufacturer's protocol. Cell lysates were prepared 48 h post-transfection and CAT ELISA (Roche, Indianapolis) analysis was carried out according to the manufacturer's instructions. The relative absorbance was calculated using following method:

$$Ar = \frac{Aa}{Ac}$$

Where Ar: the relative absorbance

Aa: the absorbance acquired by plate reader at 405 nm for samples dosed with RNA replicon or particles

Ac: the absorbance acquired by plate reader at 405 nm for untreated cells

Analysis of CAT expression. The expression of CAT protein from CAT replicon RNA or 1 μm BSA PRINT particles containing 1 wt % CAT replicon RNA as cargo was compared. Typically, 2×10⁴ Vero cells were plated into 24 well tissue cultured treated plates 18-24 h prior to assay. Vero cells were transfected with CAT RNA replicon or PRINT BSA particles containing lwt % CAT replicon RNA utilizing the TransIT® mRNA transfection kit (Mirus Bio, Madison, Wis.) following the manufacturer's protocol. Briefly, to 100 μL of Opti-MEM® I Reduced-Serum Medium, 2 μg of particles, 2 μL of TransIT and 2 μL of boost were added and mixed through pipetting. The mixture was subsequently incubated with Vero cells for 4 h at 37° C. and the non-internalized particles were removed. The cells were further incubated for another 48 h at 37° C. to allow CAT protein to express. Cell lysates were prepared 48 h post-transfection and CAT ELISA (Roche, Indianapolis) analysis was carried out according to the manufacturer's instructions. The amount of CAT protein generated was calculated based on a standard curve from 2, 1, 0.5, 0.25, 0.125 and 0 ng/mL of CAT protein.

Immunofluorescence Microscopy. Vero cells plated at on cover slips in 6-well dishes and grown for 24 hours. Cells were treated with particles for 48 h. Cells were then washed with PBS and fixed with 4% Para formaldehyde in PBS for 10 min at room temperature. Cells were permeablized with 0.1% triton-X100 in PBS for 3 min and incubated and washed in PBS for 3 times. Samples were then blocked in 5% normal serum in 1% BSA/0.2% triton X-100/PBS overnight at 4° C. Cells were then incubated in primary antibody abcam (CAT#ab50151) for 1 hr at room temperature, cells were then washed with PBS and incubated in secondary Alexa Fluor® 546 goat anti-rabbit IgG (H+L) (A11010, invitrogen) for 1 hr at RT in dark. Washed twice in PBS and mounted with Fluorsave™ reagent. Samples were then analyzed by confocal microscopy. Confocal images were acquired using a Ziess 710 laser scanning confocal imaging system (Olympus) fluorescence microscope fitted with a PlanApo 60× oil objective (Olympus). The final composite images were created using Adobe Photoshop CS (Adobe Systems, San Jose, Calif.).

Analysis of Luciferase expression and GFP expression. The expression of Luciferase protein from Luciferase or GFP replicon RNA or 1 μm BSA PRINT particles containing Luciferase replicon RNA as cargo was compared. Typically, 2×10⁴ Vero cells were plated into 24 well tissue cultured treated plates 18-24 h prior to assay. Vero cells were transfected with Luciferase or GFP RNA replicon or PRINT BSA particles containing Luciferase or GFP replicon RNA utilizing the TransIT® mRNA transfection kit (Minis Bio, Madison, Wis.) following the manufacturer's protocol. Briefly, to 100 μL of Opti-MEM® I Reduced-Serum Medium, 2 μg of particles, 2 μL of TransIT and 2 μL of boost were added and mixed through pipetting. The mixture was subsequently incubated with Vero cells for 4 h at 37° C. and the non-internalized particles were removed. The cells were further incubated for another 48 h at 37° C. to allow Luciferase or GFP protein to express. Cell lysates were prepared 48 h post-transfection and Luciferase assay was carried out according to the manufacturer's instructions. Cells expressing GFP were imaged using a Ziess 710 laser scanning confocal imaging system (Olympus) fluorescence microscope fitted with a PlanApo 60× oil objective (Olympus).

The following references are incorporate herein by reference in their entirety: Reversible hydrophobic modification of drugs for improved delivery to cells, Monahan, Sean D.; Subbotin, Vladimir; Neal, Zane C.; Budker, Vladimir G.; Budker, Tatyana, U.S. Pat. Appl. Publ. (2009), US 20090074885 A1 filed 2009 Mar. 19; Targeted drug delivery by labile hydrophobic modification of drugs, Monahan, Sean D.; Budker, Vladimir G.; Neal, Zane C.; Subbotin, Vladimir, U.S. Pat. Appl. Publ. (2005), US 20050054612 A1 filed 2005 Mar. 10; Protein and peptide delivery to mammalian cells in vitro, Monahan, Sean D.; Budker, Vladimir G.; Ekena, Kirk; Nader, Lisa, U.S. Pat. Appl. Publ. (2004), US 20040151766 A1 filed 2004 Aug. 5; J. Med. Chem. 1993, 36, 3087-3097 3087. Catalytic Functionalization of Polymers: A Novel Approach to Site Specific Delivery of Misoprostol to the Stomach, Samuel J. Tremont, Paul W. Collins, William E. Perkins, Rick L. Fenton, Denis Forster, Martin P. McGrath; Grace M. Wagner, Alan F. Gasiecki, Robert G. Bianchi, Jacquelyn J. Casler, Cecile M. Ponte, James C. Stolzenbach, Peter H. Jones, Janice K. Gard, and William B. Wise, Monsanto Corporate Research, 800 North Lindbergh Boulevard, St. Louis, Mo., 63167, and Searle Discovery Research, 4901 Searle Parkway, Skokie, Ill. 60077.

Throughout this specification and the claims, the words “comprise,” “comprises,” and “comprising” are used in a non-exclusive sense, except where the context requires otherwise.

As used herein, the term “about,” when referring to a value is meant to encompass variations of, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A compound of the formula: 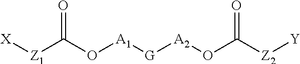
wherein,
X and Y are each independently selected from the group consisting of a polymerizable moiety and a leaving group;
G is —S—S—;
A₁ and A₂ are each independently selected from the group consisting of —C(R^{a}R^{b})—C(R^{c}R^{d})—, wherein R^{a}, R^{b}, R^{c} and R^{d} are each independently selected from the group consisting of hydrogen, C₁₋₆ alkyl and hydroxyl; and 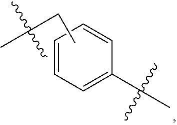
wherein G is adjacent to the ring; and
Z₁ and Z₂ are each independently selected from the group consisting of NH, O or S.

2. The compound of claim 1, wherein said polymerizable moiety is selected from the group consisting of —CH═CH₂, —C(CH₃)═CH₂, and —CH═CH—O—CH═CH₂.

3. The compound of claim 1, wherein X is a leaving group selected from the group consisting of triflate, tosyl, Cl,

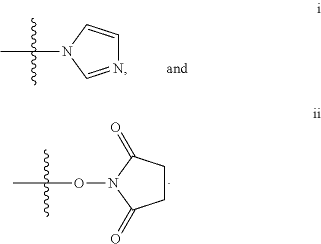 [SMILES: *n1ccnc1 ; "and" ; *ON1C(=O)CCC1=O]

4. The compound of claim 3, wherein Y is a leaving group selected from the group consisting of group selected from the group consisting of triflate, tosyl, Cl,

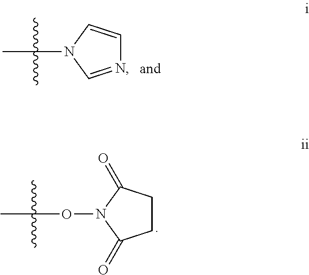 [SMILES: *n1ccnc1 ; *ON1C(=O)CCC1=O]

5. The compound of claim 1, wherein A₁ and A₂ are both —C(R^{a}R^{b})—C(R^{c}R^{d})—, wherein R^{a}, R^{b}, R^{c} and R^{d} are each hydrogen.

6. The compound of claim 5, wherein said compound is of the formula:

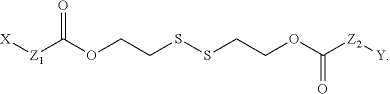

7. The compound of claim 6, wherein at least one of Z₁ and Z₂ is O or N.

8. The compound of claim 6, wherein Z₁ and Z₂ are both O or N.

9. The compound of claim 6, having one of the following structures:

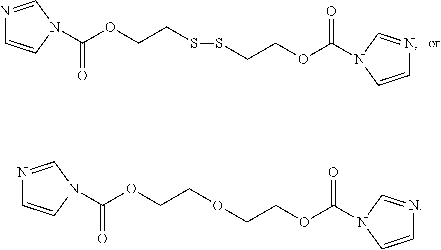 [SMILES: O=C(OCCSSCCOC(=O)n1ccnc1)n1ccnc1 ; O=C(OCCOCCOC(=O)n1ccnc1)n1ccnc1]

10. A conjugate of the formula: 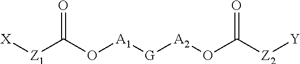
wherein,
X is a drug, a biomolecule, a polymer or a particle;
Y is selected from the group consisting of a polymerizable moiety, a leaving group, a drug, a biomolecule, a polymer and a particle;
G is —S—S—;
A₁ and A₂ are each independently selected from the group consisting of —C(R^{a}R^{b})—C(R^{c}R^{d})—, wherein R^{a}, R^{b}, R^{c} and R^{d} are each independently selected from the group consisting of hydrogen, C₁₋₆ alkyl and hydroxyl; and 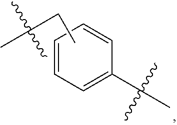
wherein G is adjacent to the ring; and
Z₁ and Z₂ are each independently selected from the group consisting of NH, O or S.

11. The conjugate of claim 10, wherein X is a biomolecule selected from the group consisting of a lipid, a protein, oligonucleotides, siRNA, RNA replicon, cDNA, nucleic acids, morpholinos, peptide nucleic acids, polysaccharides, sugars and enzymes.

12. The conjugate of claim 10, wherein A₁ and A₂ are both —C(R^{a}R^{b})—C(R^{c}R^{d})—, wherein R^{a}, R^{b}, R^{c} and R^{d} are each hydrogen and said conjugate is of the formula:

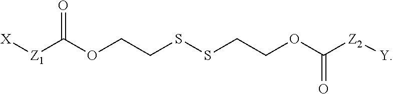

13. The conjugate of claim 10, wherein Y is a drug, a biomolecule, a polymer or a particle.

14. The conjugate of claim 10, wherein Y is a biomolecule selected from the group consisting of a lipid, a protein, oligonucleotides, siRNA, RNA replicon, cDNA, nucleic acids, morpholinos, peptide nucleic acids, polysaccharides, sugars and enzymes.

15. The conjugate of claim 10, wherein Y is a polymer selected from the group consisting of PEG.

16. The conjugate of claim 10, wherein Y is a polymerizable moiety selected from the group consisting of —CH═CH₂, —C(CH₃)═CH₂, and —CH═CH—O—CH═CH₂.

17. The conjugate of claim 10, wherein Y is a leaving group selected from the group consisting of group selected from the group consisting of triflate, tosyl, Cl,

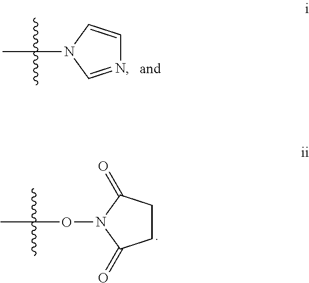 [SMILES: *n1ccnc1 ; *ON1C(=O)CCC1=O]

18. A conjugate of one of the following formulae: 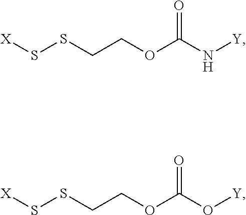
wherein X is 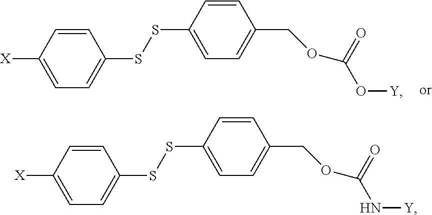
wherein X is 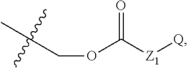
wherein,
Z₁ is O, N or S and Q is a polymerizable moiety or a leaving group; and
Y is a drug, a biomolecule, a polymer or a particle.

19. (canceled)

20. (canceled)

21. The conjugate of claim 18 selected from the group consisting of

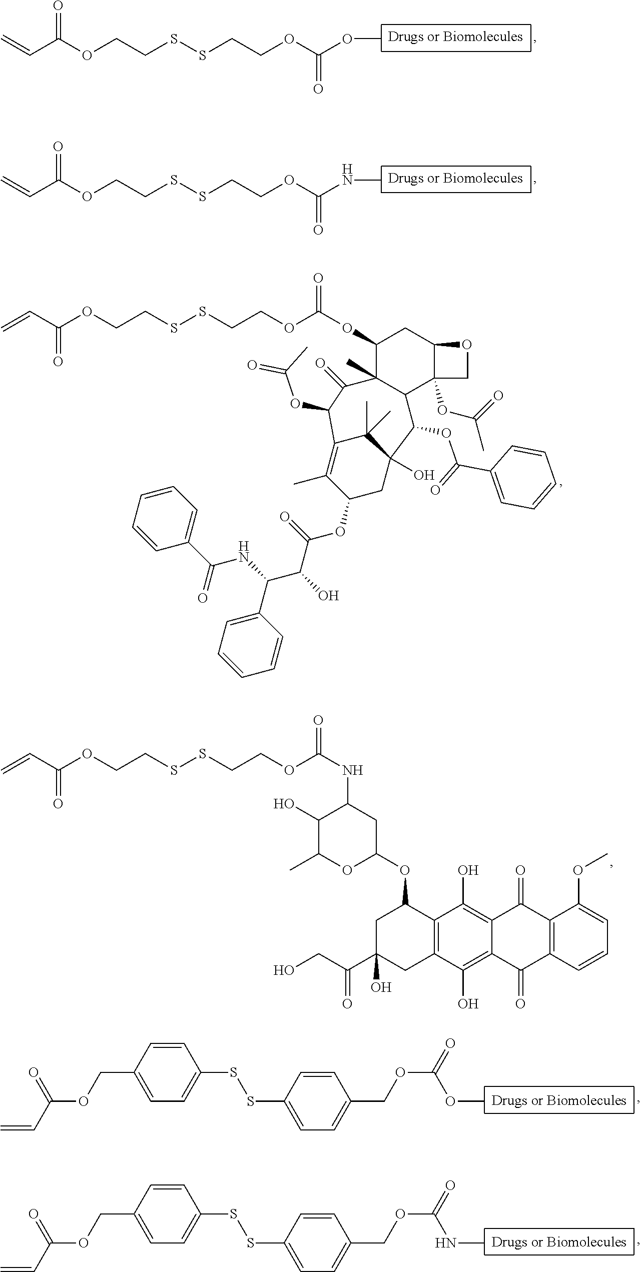

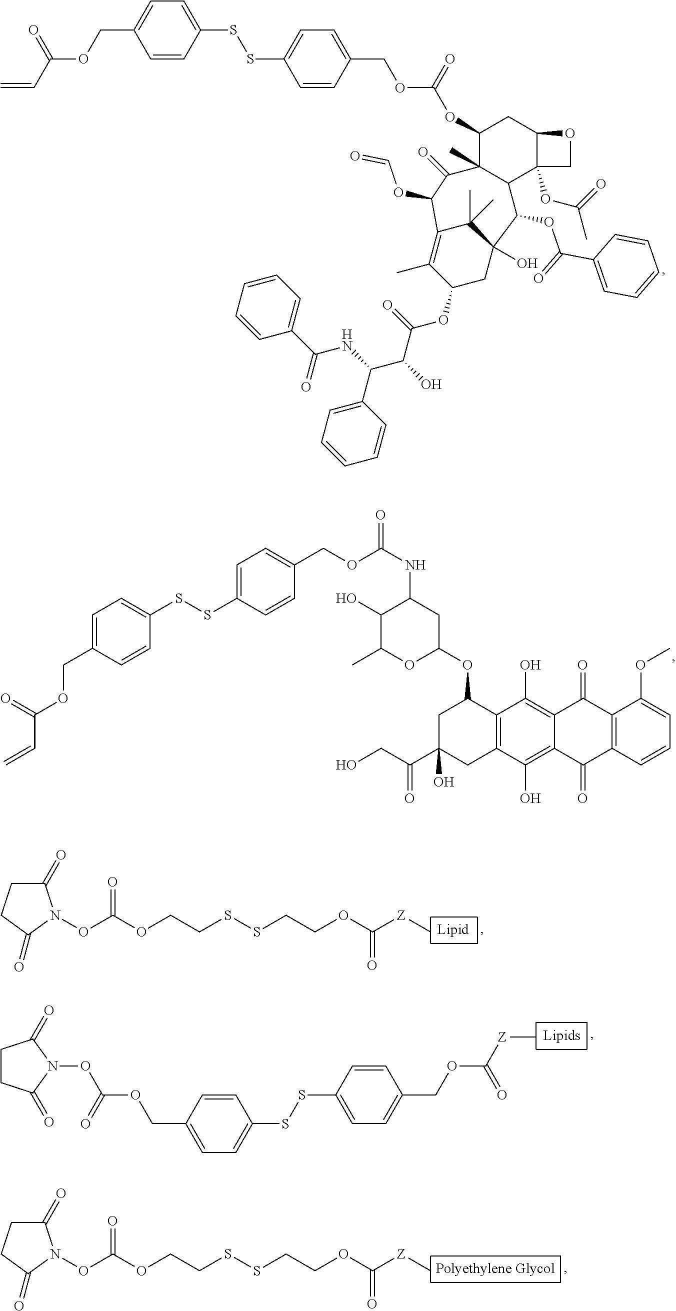

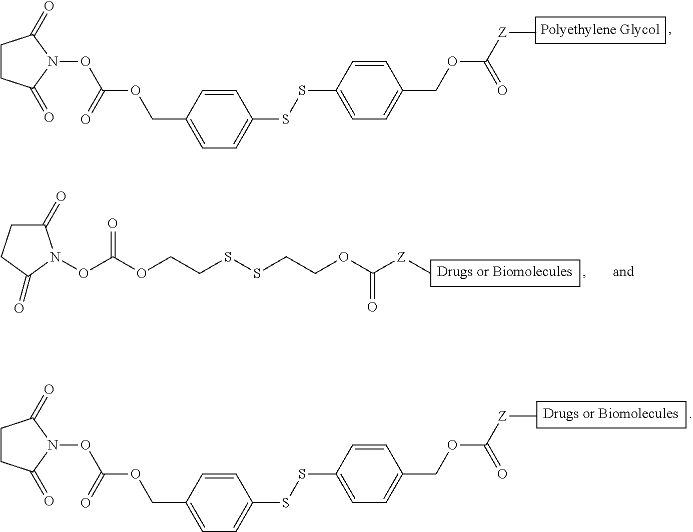

22. (canceled)

23. (canceled)

24. (canceled)

25. (canceled)

26. (canceled)
